(19) United States
(12) Patent Application Publication
(10) Pub. No.: US 2024/0006051 A1
(43) Pub. Date: Jan. 4, 2024

(54) SYSTEMS AND METHODS TO PREDICT AN INDIVIDUALS MICROBIOME STATUS AND PROVIDE PERSONALIZED RECOMMENDATIONS TO MAINTAIN OR IMPROVE THE MICROBIOME STATUS

(71) Applicant: SOCIETE DES PRODUITS NESTLE S.A., Vevey (CH)

(72) Inventor: SHAILLAY KUMAR DOGRA, Epalinges (CH)

(21) Appl. No.: 18/254,058

(22) PCT Filed: Nov. 24, 2021
(86) PCT No.: PCT/EP2021/082743

§ 371 Date: May 23, 2023

Related U.S. Application Data
(60) Provisional application No. 63/228,668, filed on Aug. 3, 2021.

(30) Foreign Application Priority Data
Nov. 24, 2020 (EP) 20209382.9

(51) Int. Cl. G16H 20/60; G16H 10/20; A23L 33/00

(52) U.S. Cl. CPC G16H 20/60; G16H 10/20; A23L 33/30

(57) ABSTRACT

The present invention relates to systems and methods for predicting individual microbiome status and for providing personalized recommendations to maintain or improve the microbiome status. In several embodiments of the invention, the individual microbiome features are clustered based on their responses to a questionnaire. In several embodiments, the methods are implemented by a computer system. In several embodiments of the invention, personalized recommendations and dietary advice are given to the individual to maintain or improve said individual's microbiome status.

### FIELD OF THE INVENTION

The present invention relates to systems and methods for predicting an individual's microbiome status and for providing personalized recommendations to maintain or improve the microbiome status. In several embodiments of the invention, the individual microbiome features are clustered based on their responses to a questionnaire. In several embodiments, the methods are implemented by a computer system. In several embodiments of the invention, personalized recommendations and dietary and nutrition advice are given to the individual to maintain or improve said individual's microbiome status.

### BACKGROUND TO THE INVENTION

The gut microbiota is host to trillions of microorganisms, mainly bacteria living in the intestine, particularly in the colon. Alterations in the composition and functions of gut microbiota are associated with many diseases and conditions such as Irritable Bowel Syndrome, Inflammatory Bowel Disease, allergy, diabetes, cancer, asthma, and obesity (Dogra S K, et al. Front. Microbiol. 2020).

The composition of the microbiota is influenced by various extrinsic factors such as diet, geographic location, ethnicity, exercise/physical activity, antibiotics use and use of other types of medication (Rothschild D, et al. Nature. 2018). However, these extrinsic factors do not reliably predict the microbiome status of an individual, as an individual's microbiome at any point in their lifespan depends both on the intrinsic microbiome composition as well as these extrinsic factors.

As no two microbiomes are the same between individuals, there is a need for methods and systems to provide individualized recommendations for microbiome health. The solution for successful maintenance or improvement of the microbiome health requires the assessment of the microbiome status before any recommendation or advice can be given.

Methods and systems of the present invention for predicting microbiome status with respect to providing dietary and nutrition recommendations to maintain or improve a healthy microbiome is different from the prior art where microbiome status was used to predict disease outcomes such as type 2 diabetes (Reitmeier S, et al. Cell Host Microbe. 2020; Wu H, et al. Cell Metab. 2020), Post-prandial glycemic response (Zeevi D, et al. Cell. 2015), NAFLD-cirrhosis (Oh T G, et al. Cell Metab. 2020), NAFLD-fibrosis (Loomba R, et al. Cell Metab. 2017) or host variables (physiological, lifestyle and dietary characteristics) (Vujkovic-Cvijin I, et al. Nature. 2020). Simply, the input to output direction is reversed in the present invention compared to previous studies.

Current assessments of an individual's gut microbiome status are based on biological sampling—either fecal or plasma sampling, use of advanced technologies such as next generation sequencing and complex bioinformatics analyses. This is time consuming, requiring a number of processing steps including: freeze storage of the fecal sample ideally at minus 80 degrees Celsius; processing the fecal sample for DNA extraction; sequencing the extracted DNA; and complex bioinformatics processing to detect and identify the multitude of microbes presence and abundance (Jove) J, et al. Front Microbiol. 2016). In addition to the time and cost required to process biological samples, many individuals are not inclined to give their fecal samples to a lab for processing (Vandeputte D, et al. FEMS Microbiol Rev. 2017) nor even plasma samples for processing to assess microbiota diversity (Wilmanski T, et al. Nat Biotechnol. 2019).

The present invention advantageously provides non-invasive methods and systems for assessing the microbiome status in an individual which does not require a biological sample such as a fecal or plasma sample. Further, the invention provides user-friendly systems and methods for individuals to assess their microbiome status. In several embodiments, the systems and methods of the invention are useful to assist the individual to modify their diet, nutrition and lifestyle according to their microbiome status.

### SUMMARY OF THE INVENTION

The methods and systems of the present invention advantageously implement Artificial Intelligence based Machine Learning methods to assess an individual's gut microbiome status from sets of questionnaires.

One advantage of the present invention is that the individual does not need to provide a biological sample to get an estimate of their microbiome status. Instead, this is done by using predictive models based on the data provided by the user in terms of responses to a set of questionnaires in order to discern predictive features.

In several embodiments, the present invention determines the microbiome status of an individual in relation to their position within the distribution in a larger population. For example, in terms of either having it Low or notLow, High or notHigh or when combined together to determine Low, Medium, High and possibly cross-confirmed by another Low vs. High assessment, where Low, High or Low, notLow or High, notHigh are defined in various ways based on the distribution seen for a large-sized general population such as in the American Gut Project (AGP) (McDonald D, et al. mSystems. 2018) and Microba Discovery Database (MDD), Microba, Australia.

In several embodiments of the invention, the systems and methods of the invention evaluate the features from the questionnaire to extract and rank the features in order of importance for determining microbiome status.

One advantage of several embodiments of the invention is that for the microbiome status assessment, individual user's questionnaire responses are evaluated to personalize the recommendations and advice to maintain or improve the individual's microbiome status.

Another advantage of several embodiments of the invention is that the microbiome status assessment of individuals is done taking into consideration the weighting of importance of their individual features; thus, any related recommendations and suggestions to improve the microbiome status is personalized.

Various embodiments of the disclosed system display a dashboard or other appropriate user interface to a user that is customized based on the user's inputs to the questionnaire, predicted microbiome status, and personalized advise to maintain or improve the microbiome status.

In some embodiments, the disclosed system may be linked to automatically collect the required input data from activity trackers or other wearable devices such as smart watches or fitness trackers.

In some embodiments, the disclosed system may be linked to automatically collect the required input data from dietary records captured by the user in various formats such as food diary or apps that log eating records.

In various embodiments, the disclosed system may work in conjunction with a laboratory or other testing facility that generates actual data about individuals using the disclosed system. For example, in one embodiment the disclosed system enables a user to submit a biological analysis report that indicates the biomarkers of the individual's biological sample. In such embodiments, the reports from such testing and lab work may enable the system to possibly improve its recommendations.

In some embodiments, the systems and methods disclosed herein can be also used by nutritionists, health-care professionals, beyond the individual users.

Further advantages of the instant disclosure will be apparent from the following detailed description and associated figures.

### DESCRIPTION OF FIGURES

FIG. 1—Computer implemented system for microbiome status assessment

A block diagram of an example system according to one embodiment of the present disclosure.

FIG. 2—Microbiome recommendation system

A schematic diagram illustrating the microbiome recommendation system with its individual components, their interfacing with each other and associated inputs, outputs from the component units.

FIG. 3A—ROC performance of a Low versus notLow model

The ROC performance of a Low versus notLow model defined on three diversity measures taken together with the definition of the bins based on quartiles. ROC for (i) Train in cross-validation mode (ii) holdout/Test set.

FIG. 3B—ROC performance of a Low versus notLow model

The ROC performance of a Low versus notLow model defined on three diversity measures taken together with the definition of the bins based on mean and standard deviation. ROC for (i) Train in cross-validation mode (ii) holdout/Test set.

FIG. 4A—ROC performance of a High versus notHigh model

The ROC performance of a High versus notHigh model defined on three diversity measures taken together with the definition of the bins based on quartiles. ROC for (i) Train in cross-validation mode (ii) holdout/Test set.

FIG. 4B—ROC performance of a High versus notHigh model

The ROC performance of a High versus notHigh model defined on three diversity measures taken together with the definition of the bins based on mean and standard deviation. ROC for (i) Train in cross-validation mode (ii) holdout/Test set.

FIG. 5A—ROC performance of a Low versus High model

The ROC performance of a Low versus High model defined on three diversity measures taken together with the definition of the bins based on quartiles. ROC for (i) Train in cross-validation mode (ii) holdout/Test set.

FIG. 5B—ROC performance of a Low versus High model

The ROC performance of a Low versus High model defined on three diversity measures taken together with the definition of the bins based on mean and standard deviation. ROC for (i) Train in cross-validation mode (ii) holdout/Test set.

FIG. 6A—Feature importance plots for a Low versus notLow model

The feature importance plots for a Low versus notLow model on three diversity measures taken together with the definition of the bins based on quartiles and input data with those features with response-rate>0.65 The top 30 features for this model are shown in these plots. (i) shows the average impact per feature on the model output sorted in the order of importance from high to low. (ii) shows in more details the impact of a feature on the model output. The color gradation from grey to black indicates low to high values for that feature. The vertical line at 0.00 defines the directionality of impact with respect to the reference class “Low”—to the left is negative impact and to the right is positive impact on the model output.

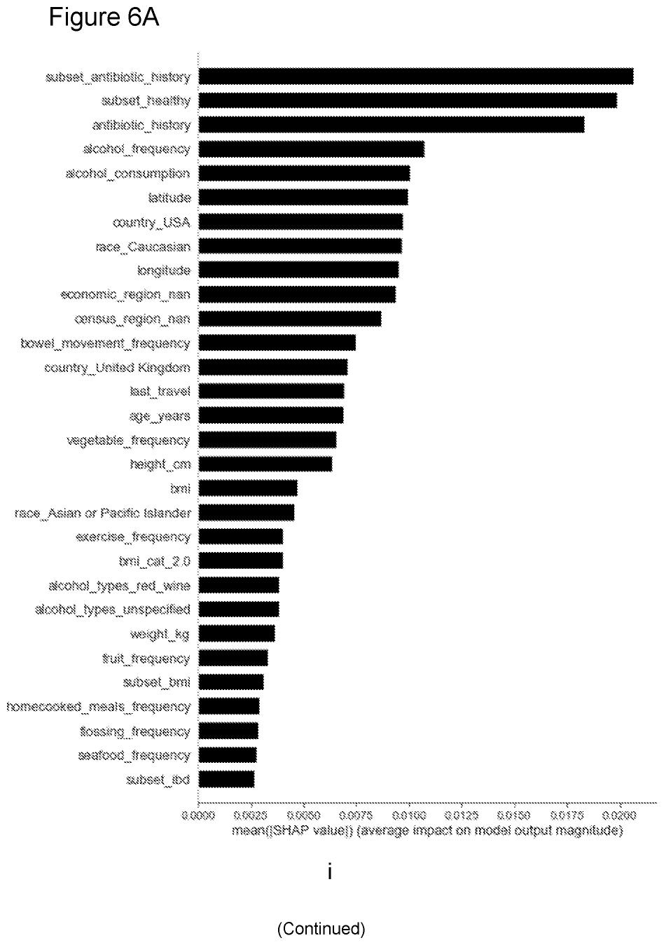

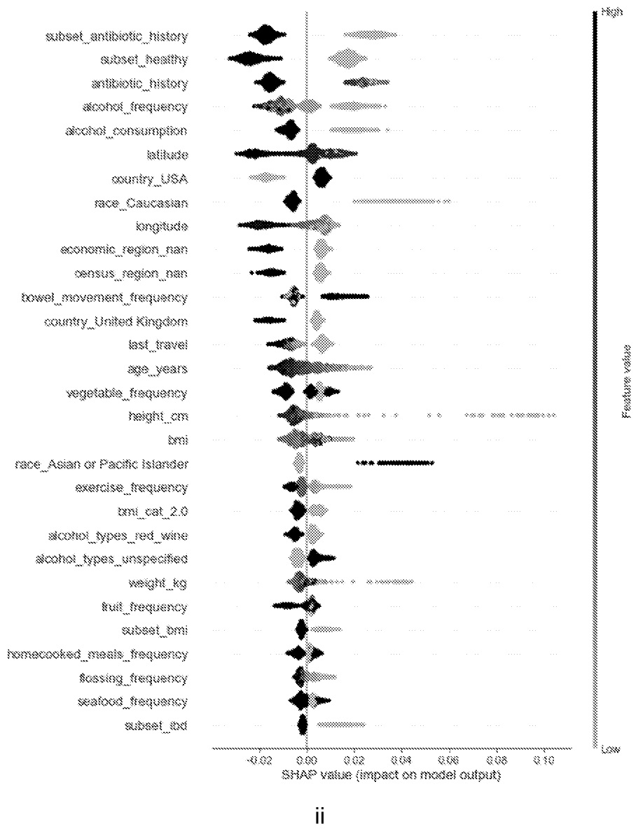

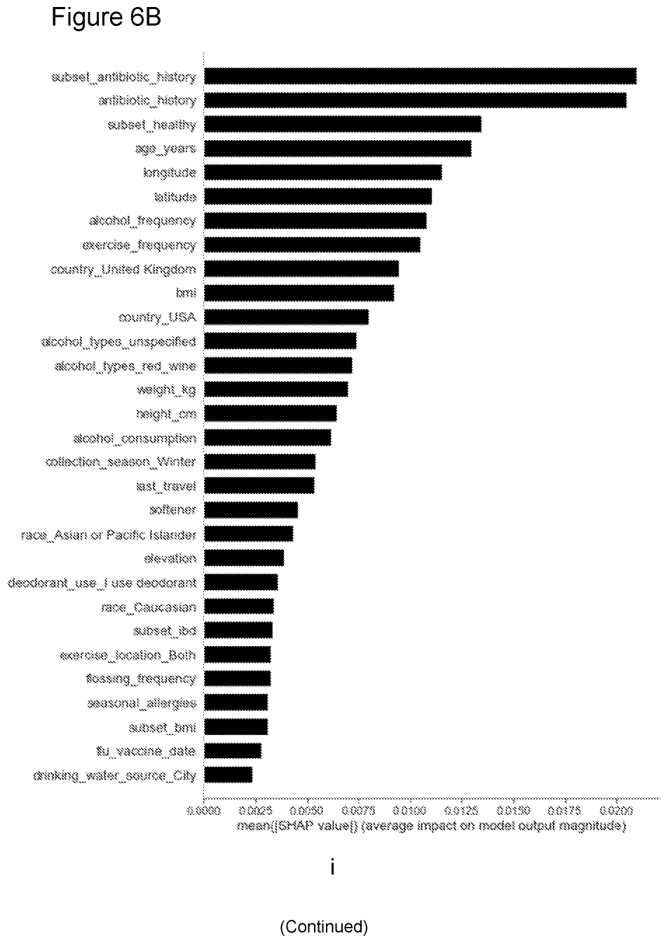

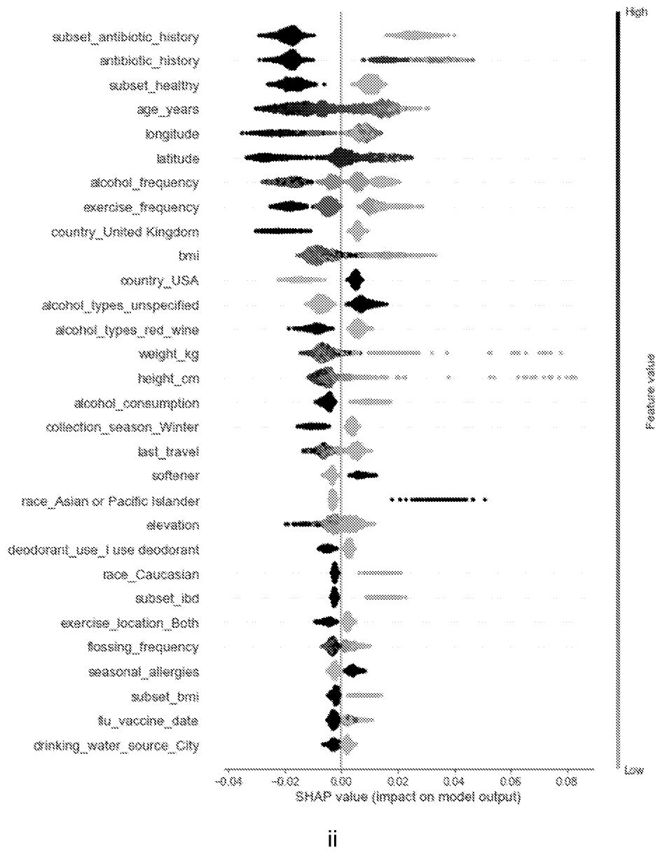

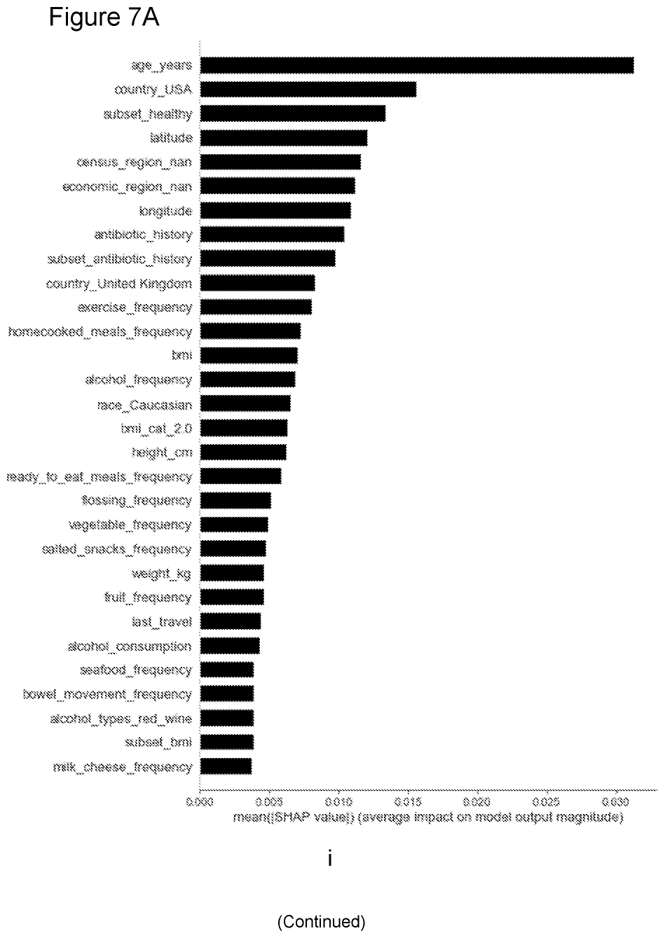

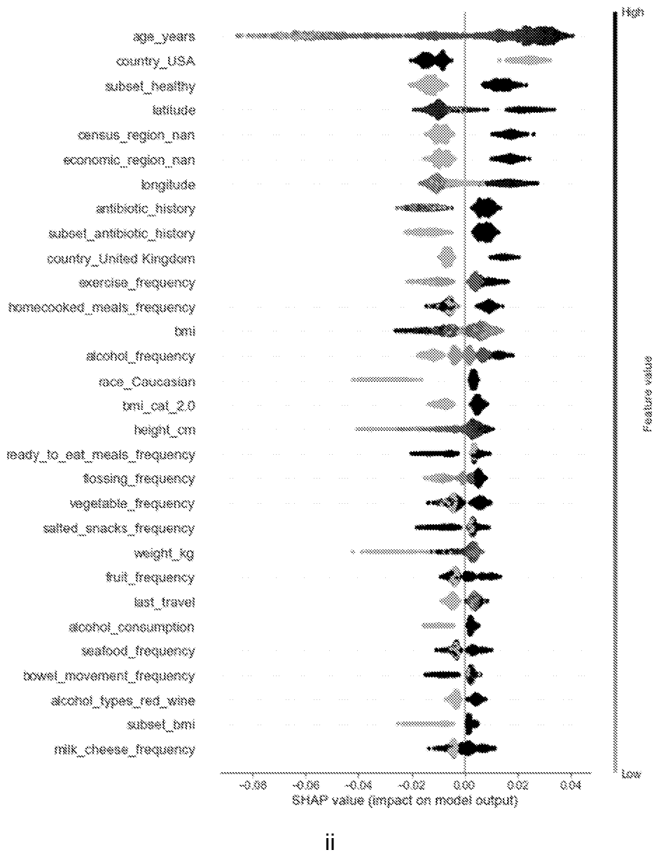

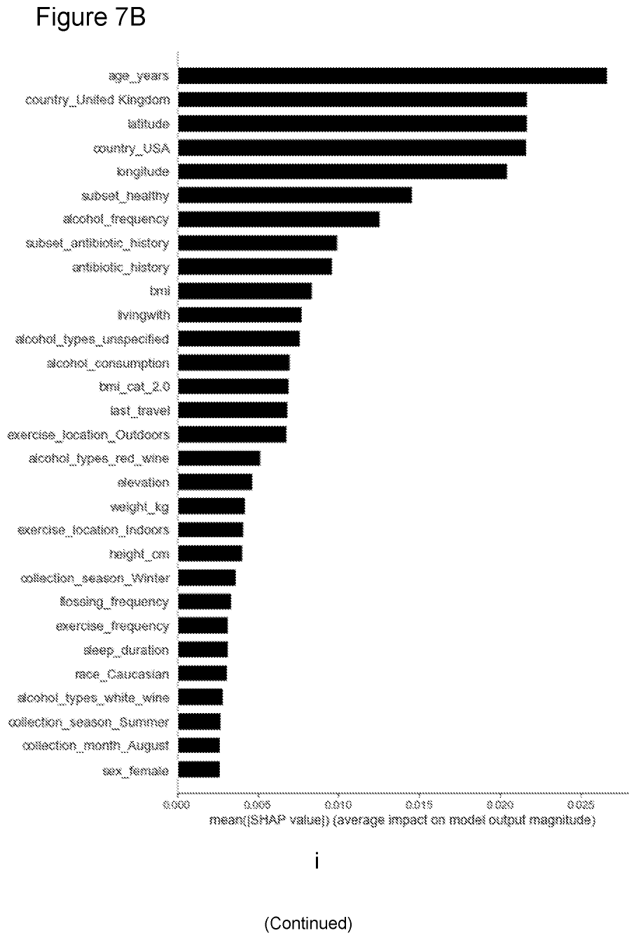

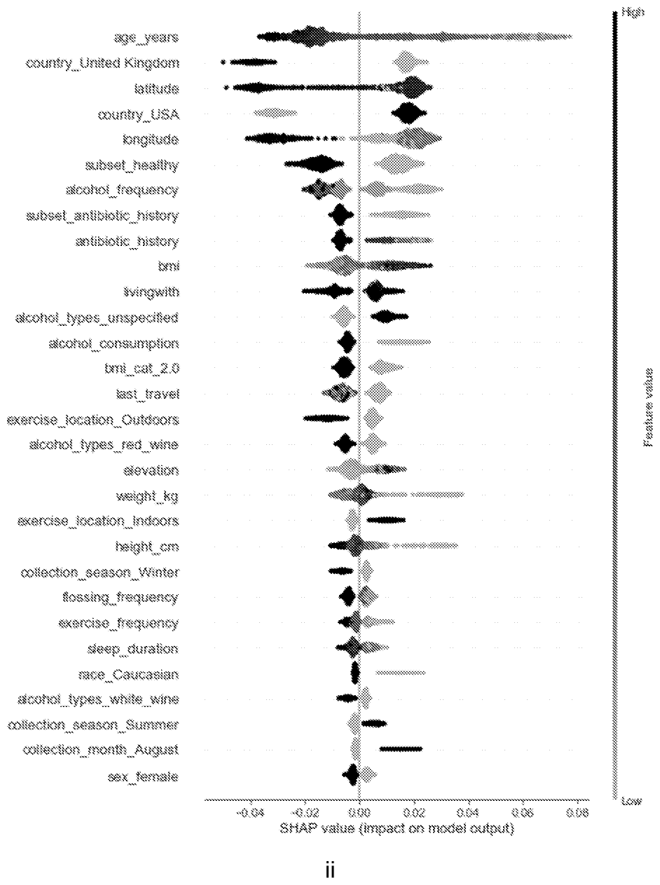

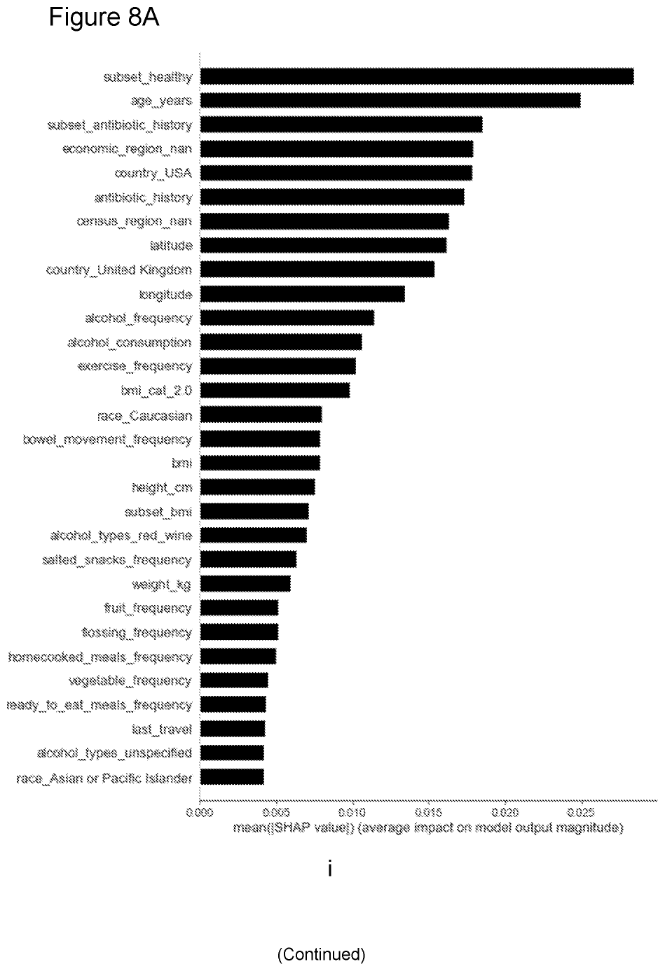

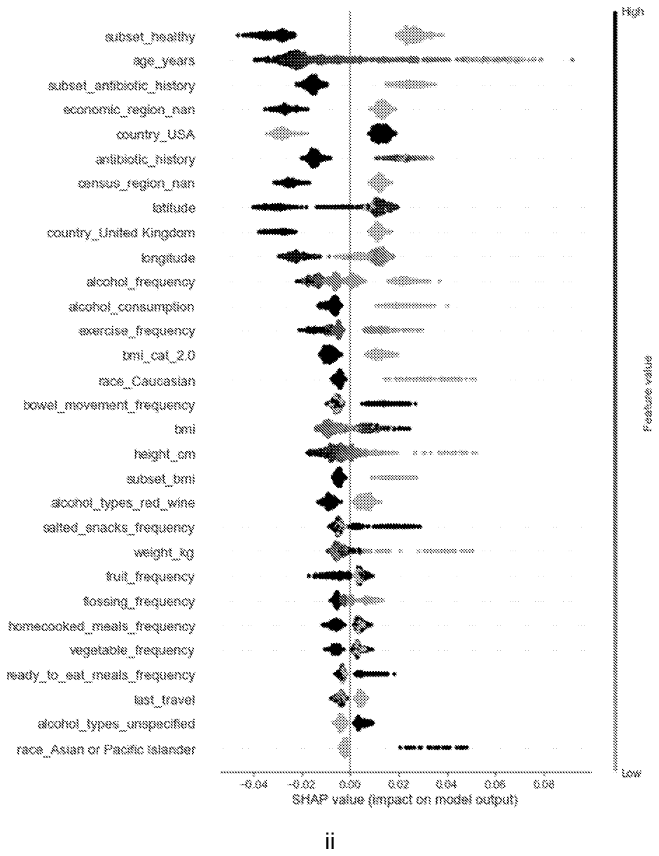

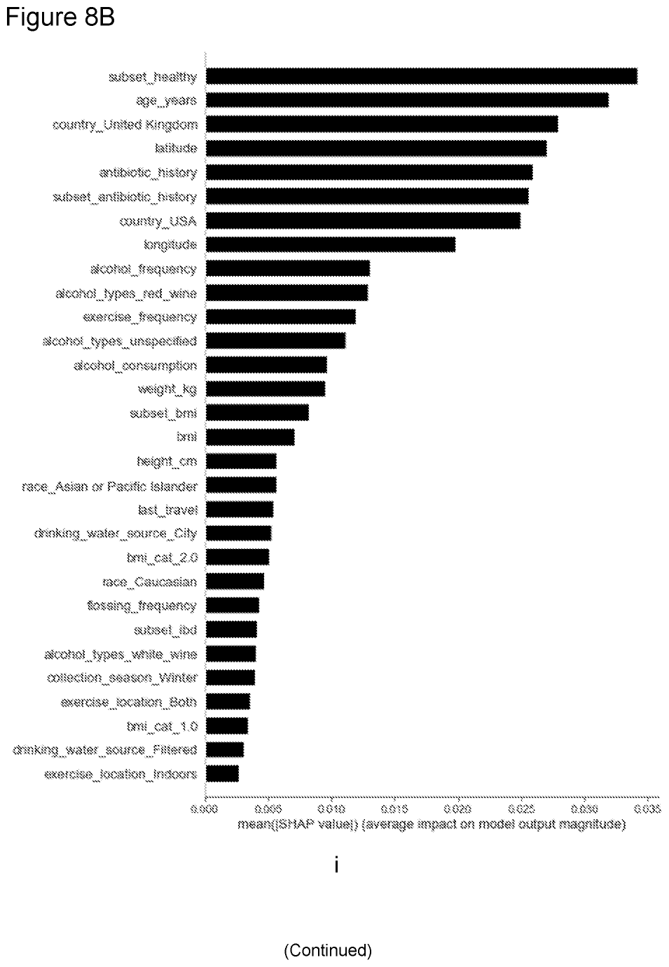

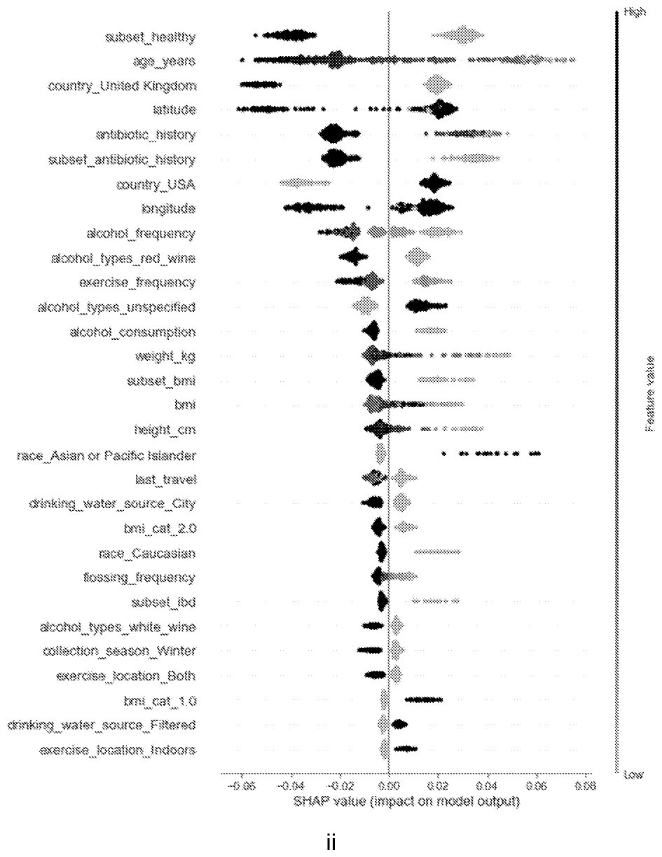

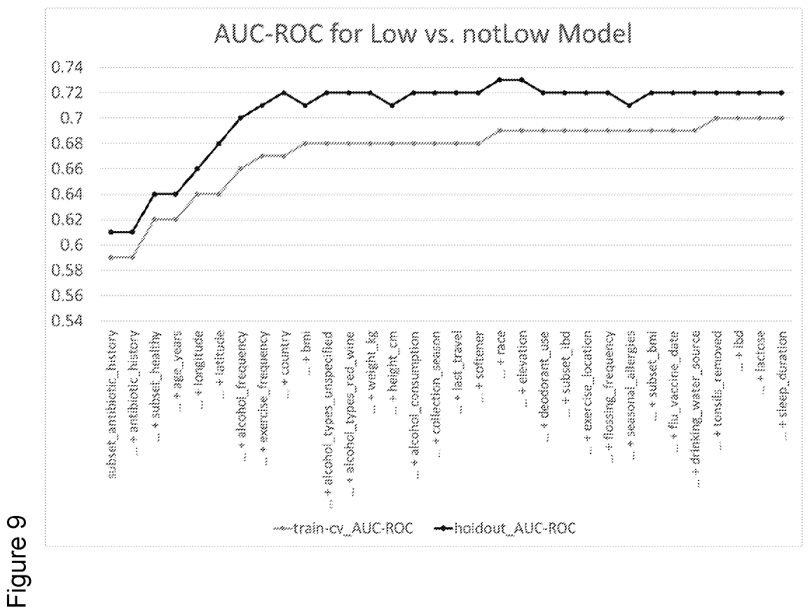

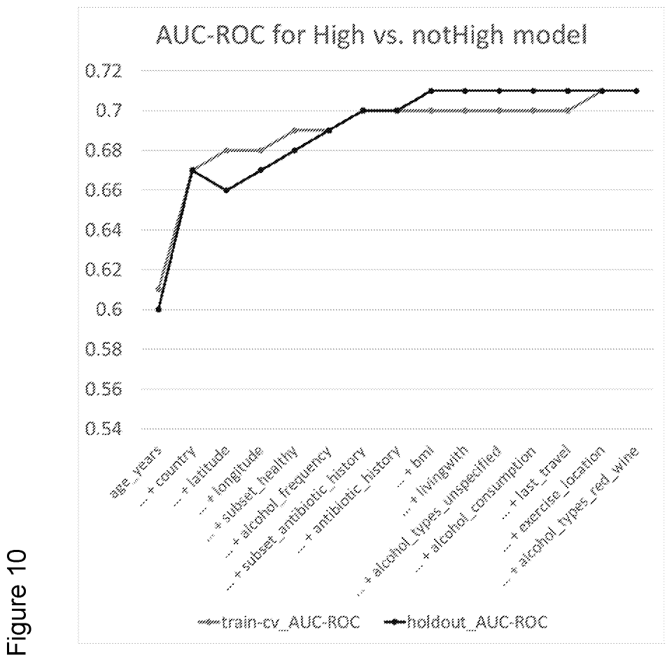

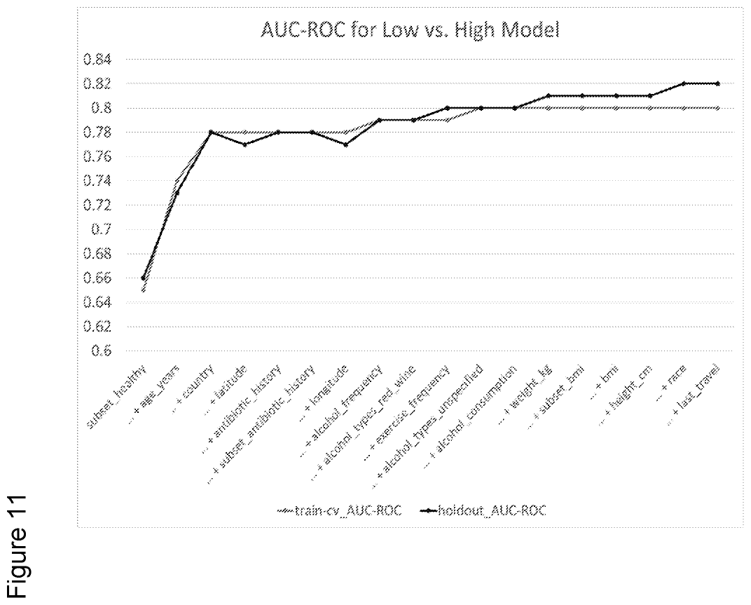

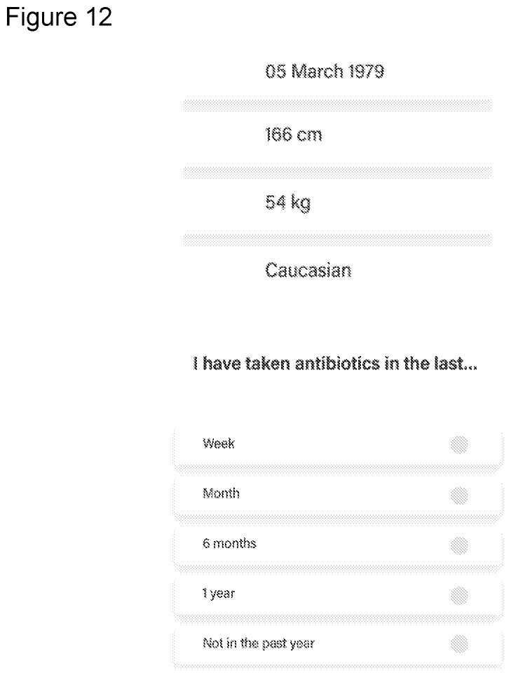

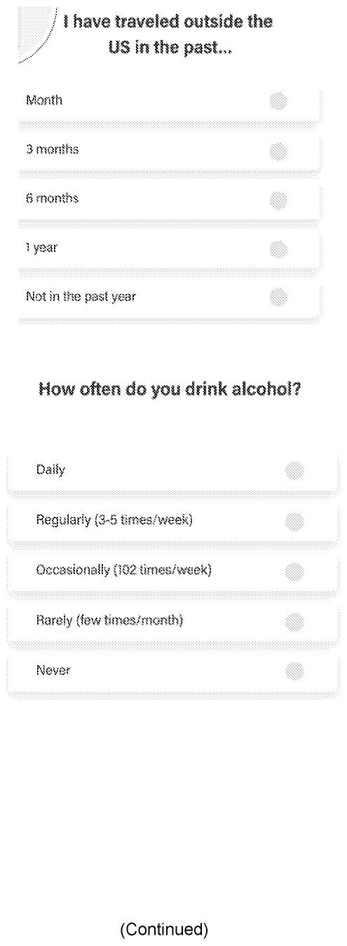

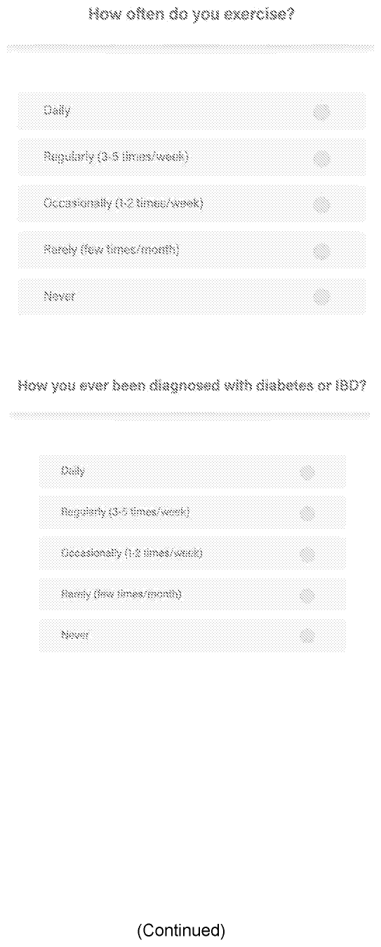

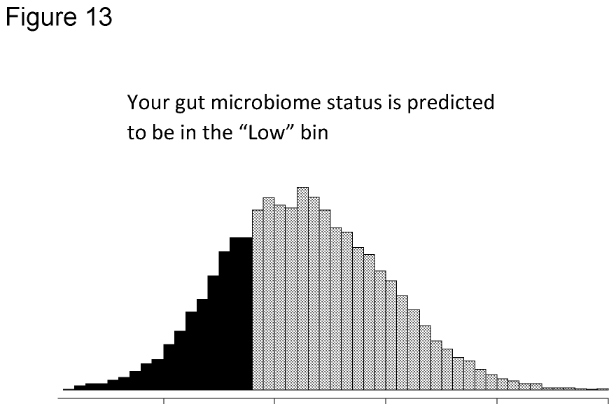

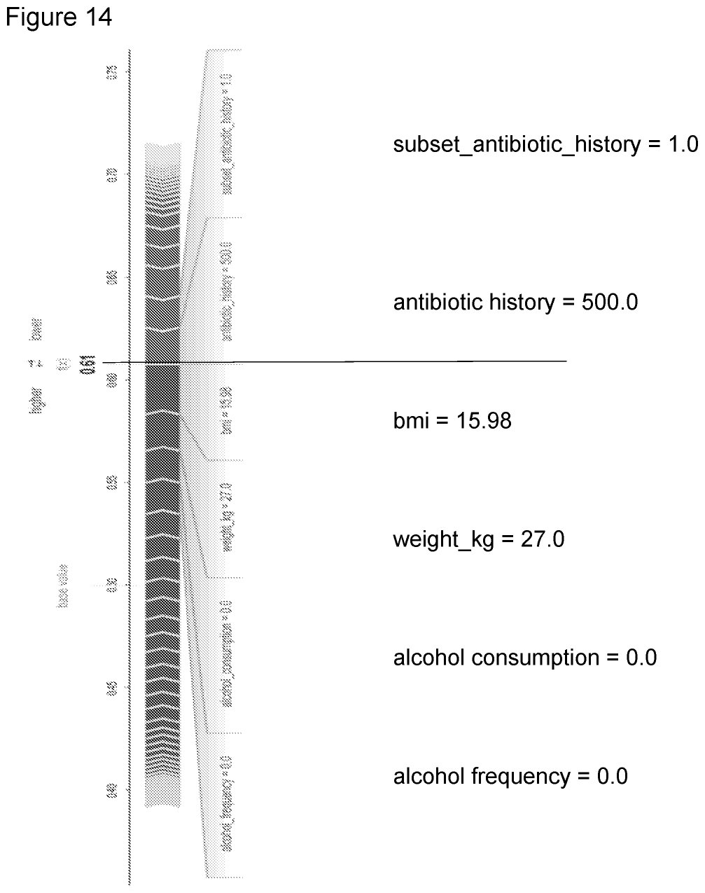

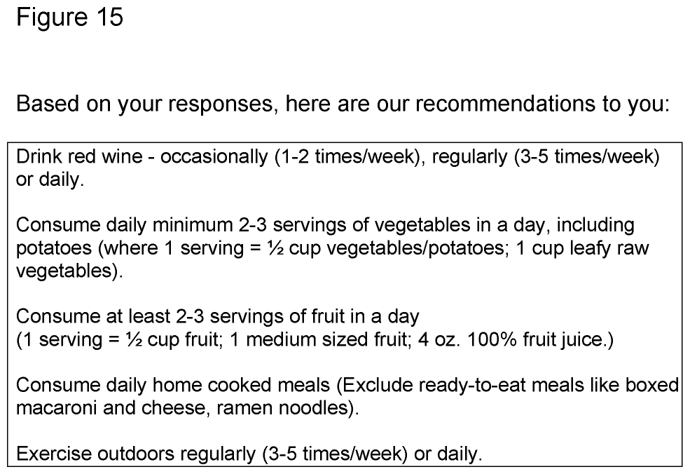

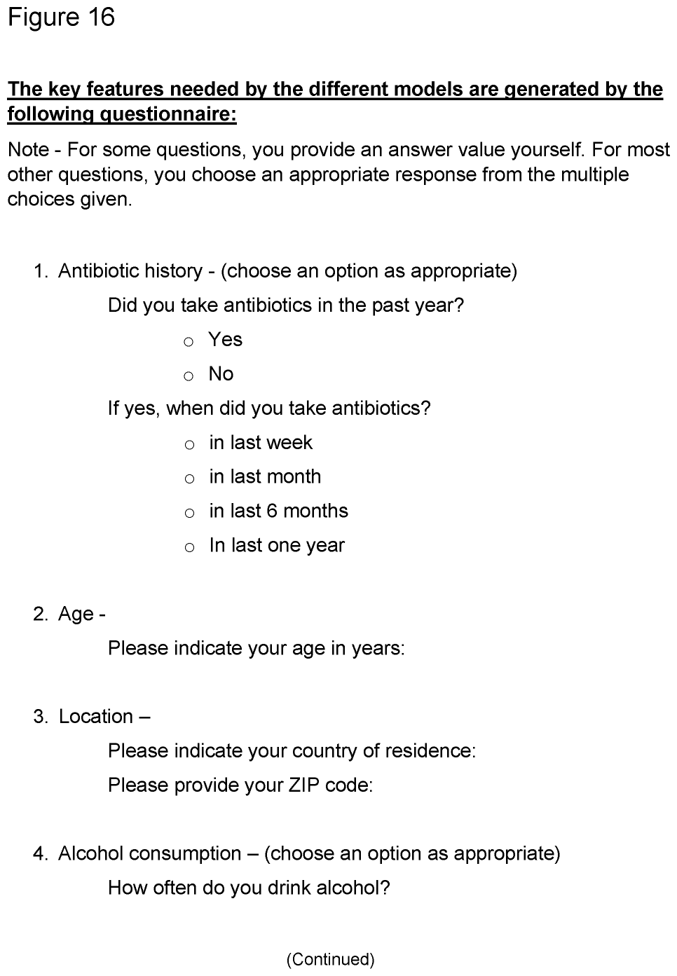

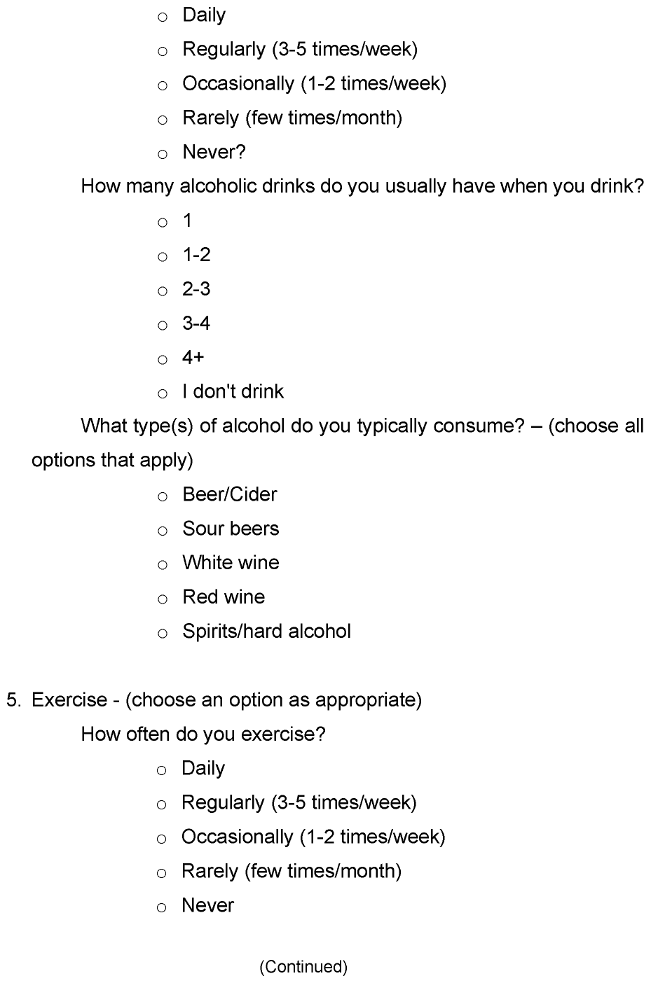

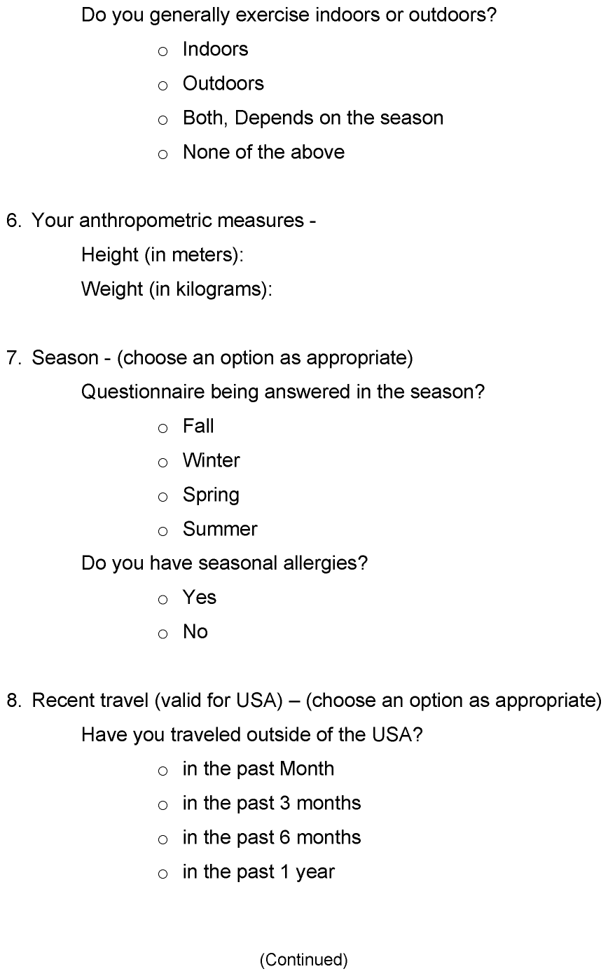

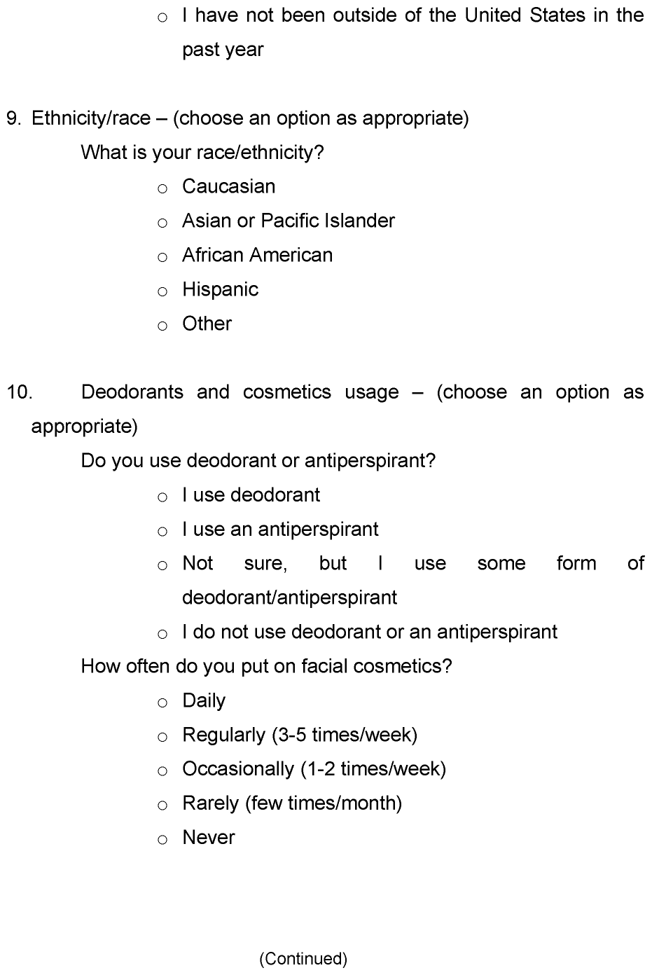

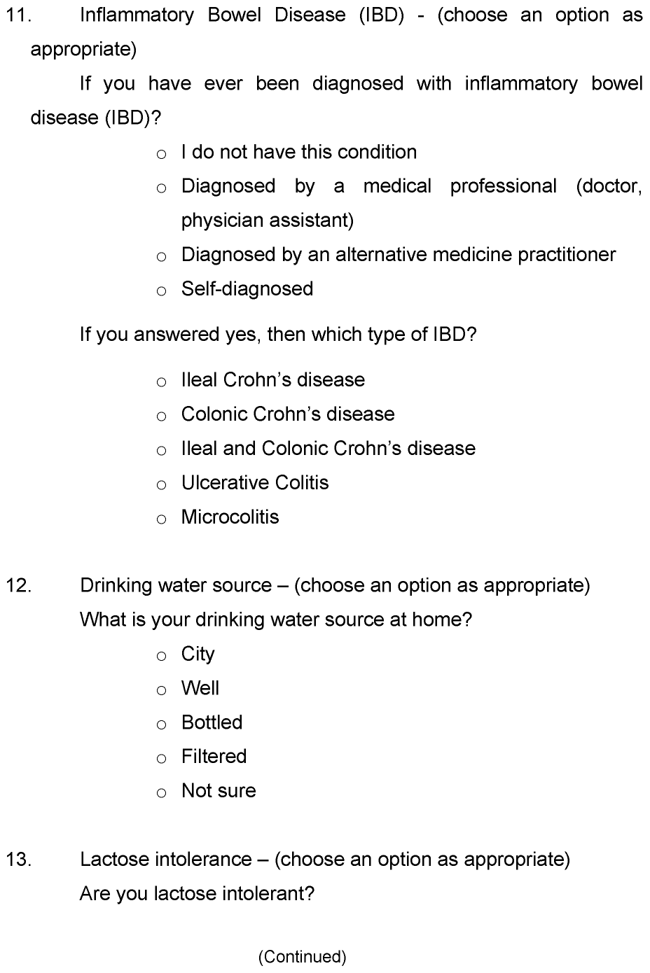

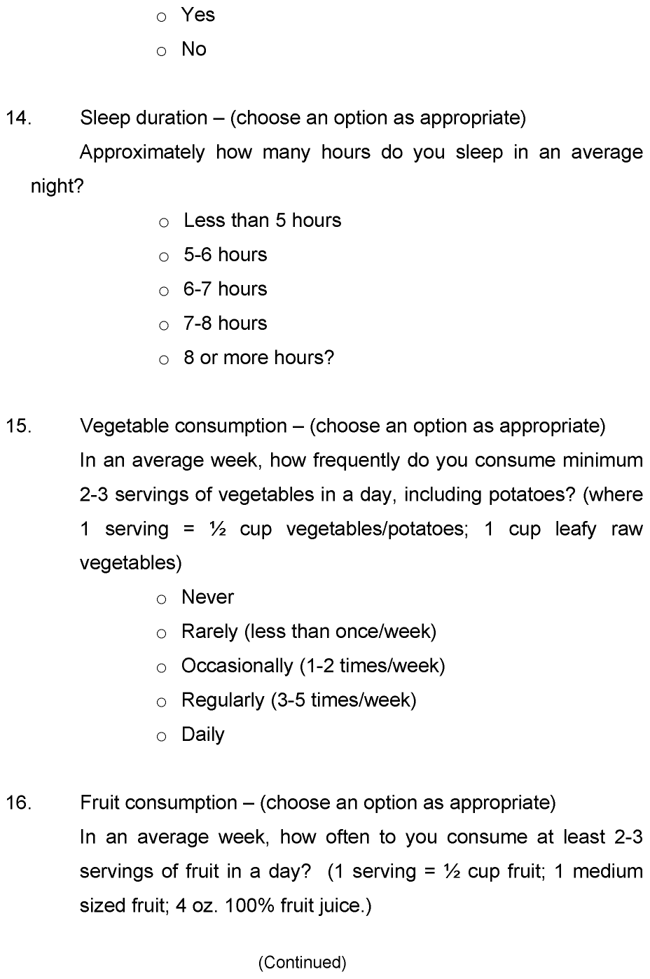

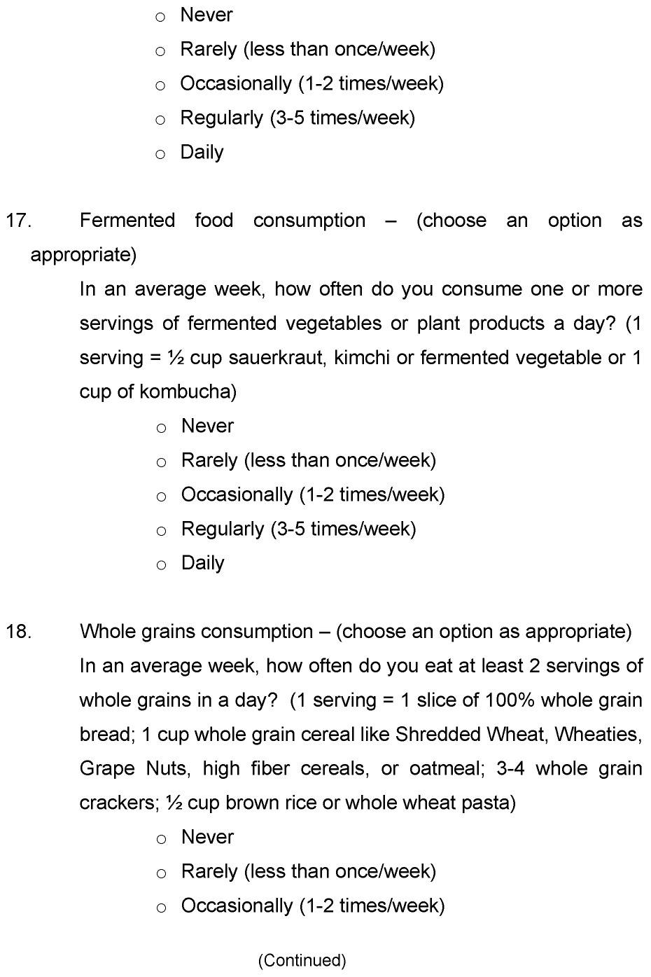

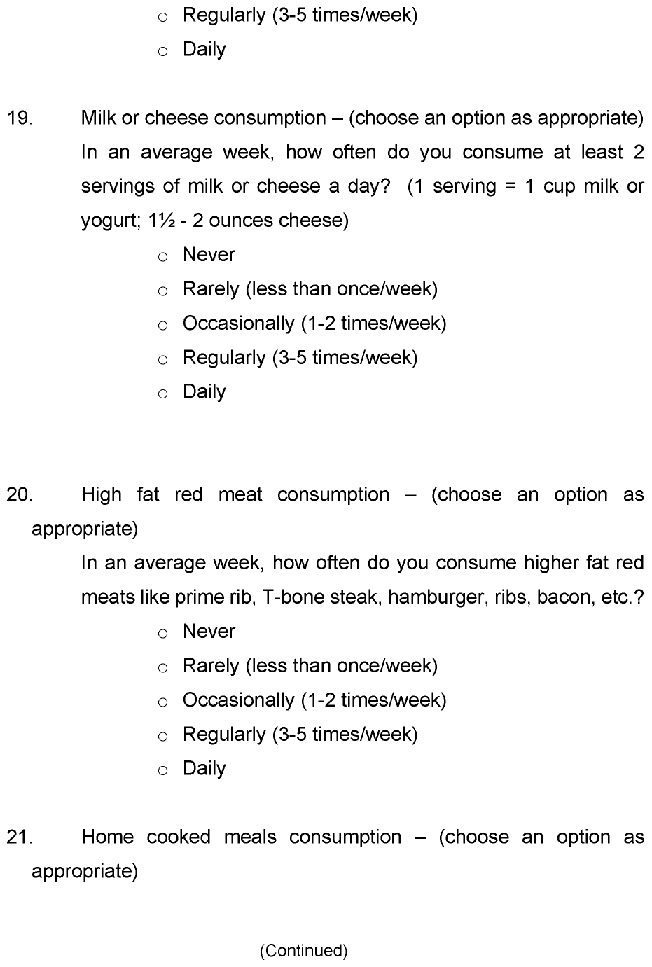

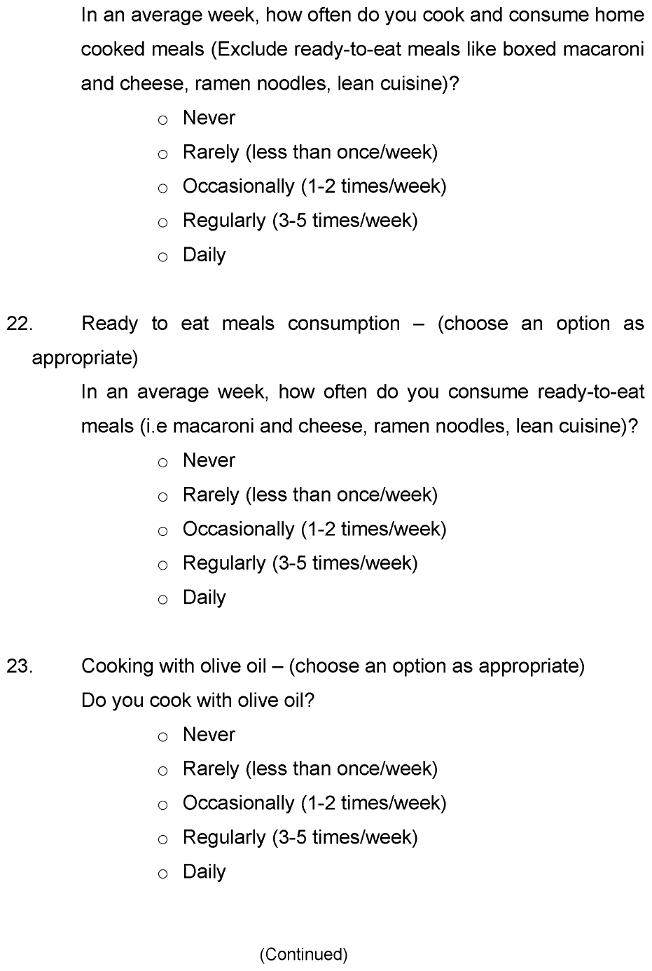

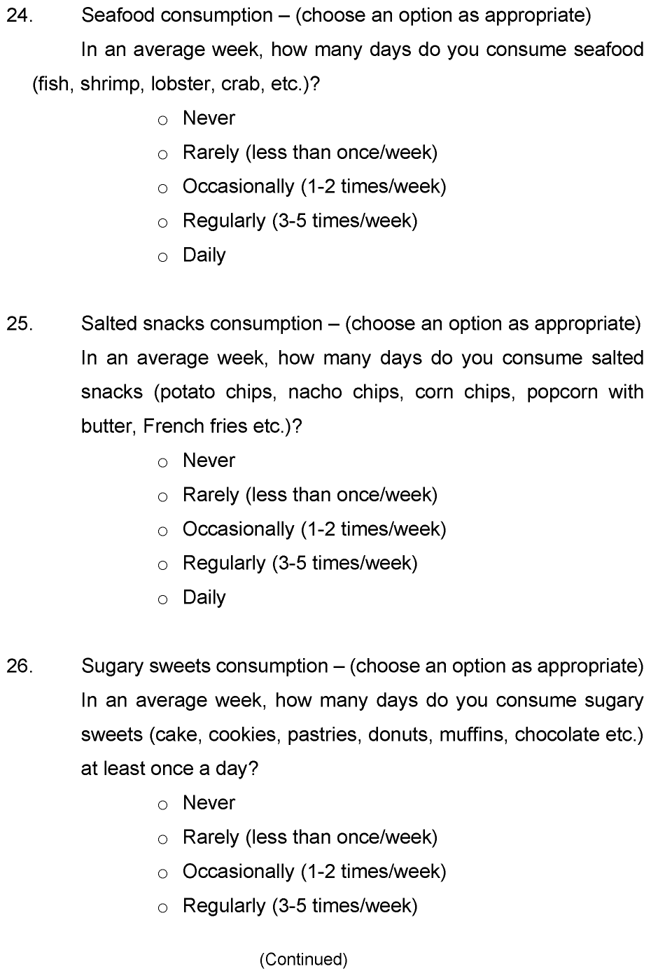

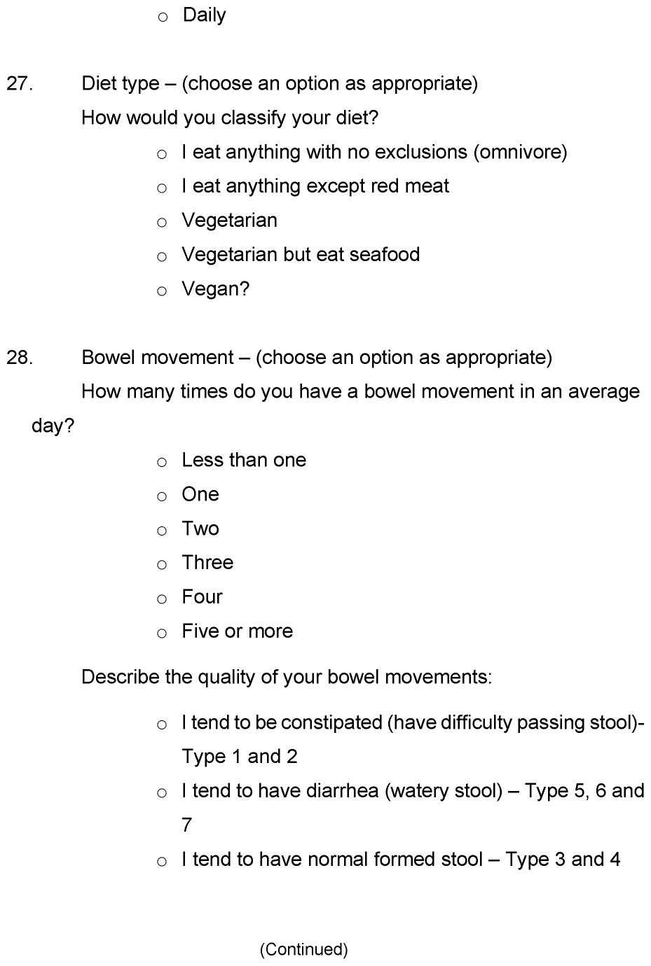

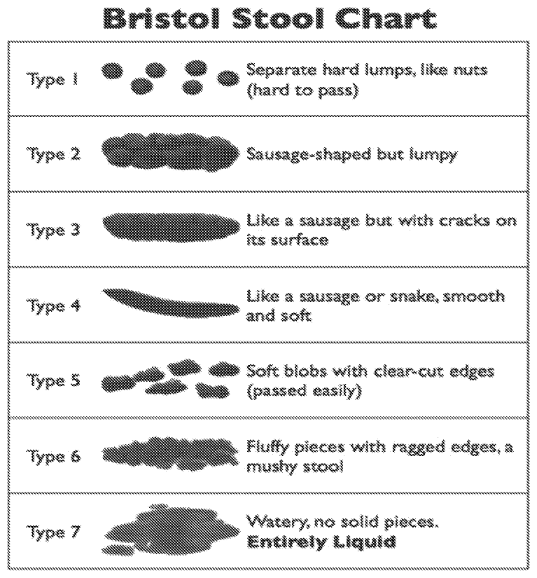

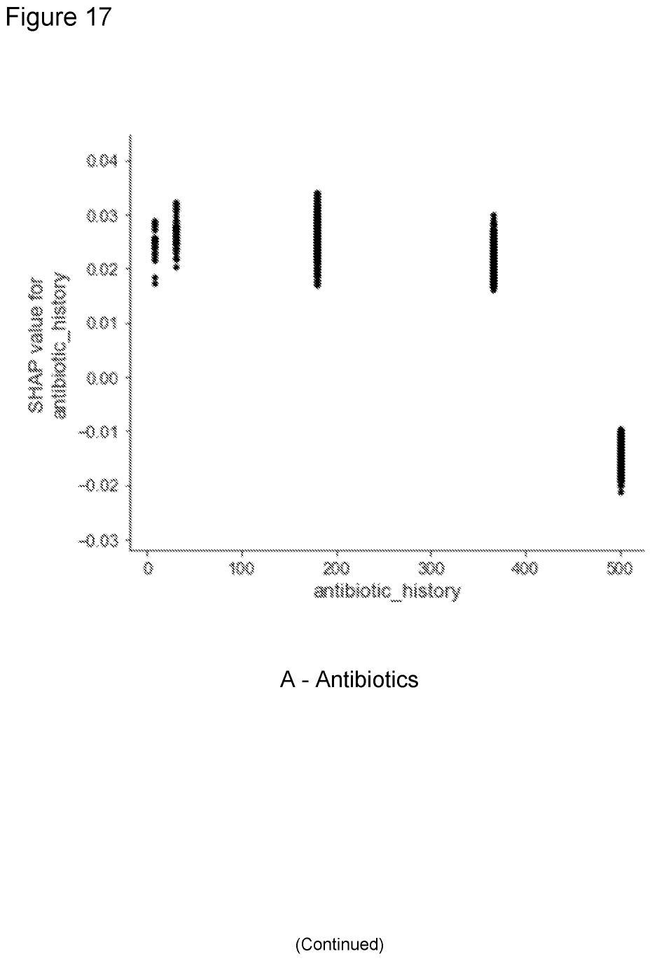

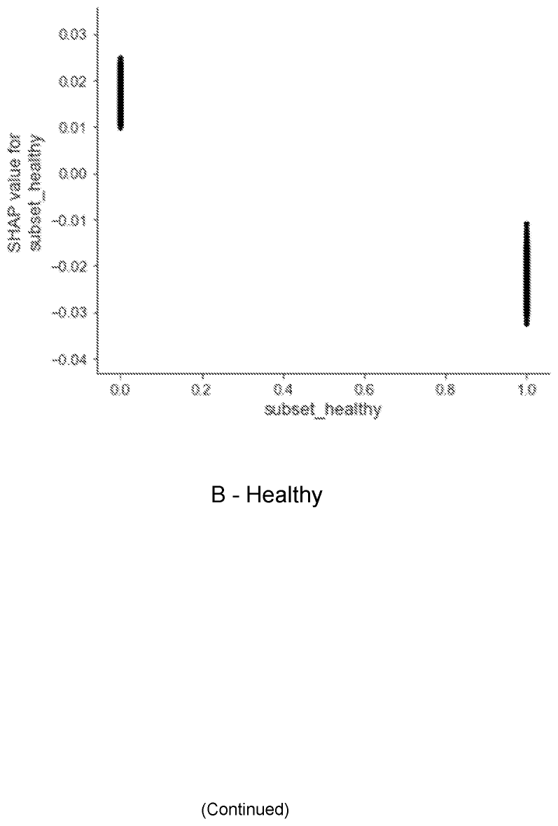

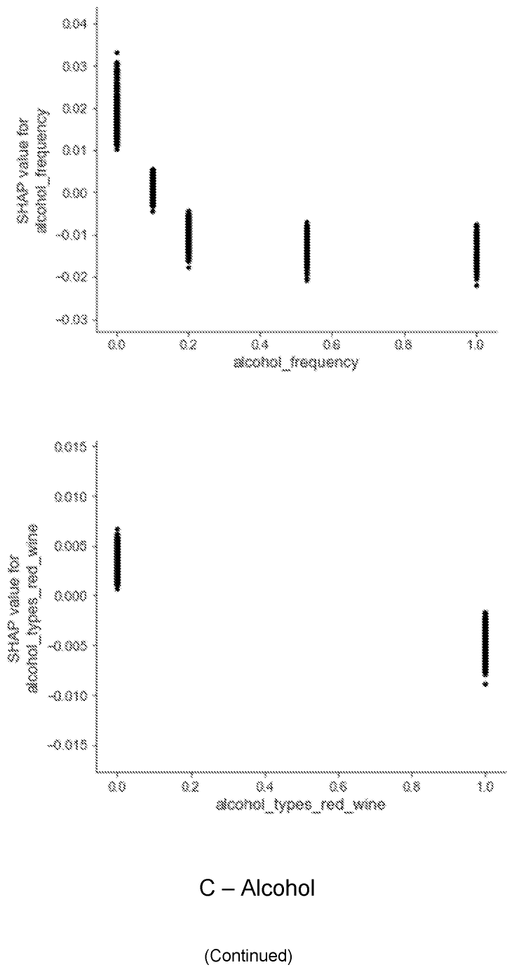

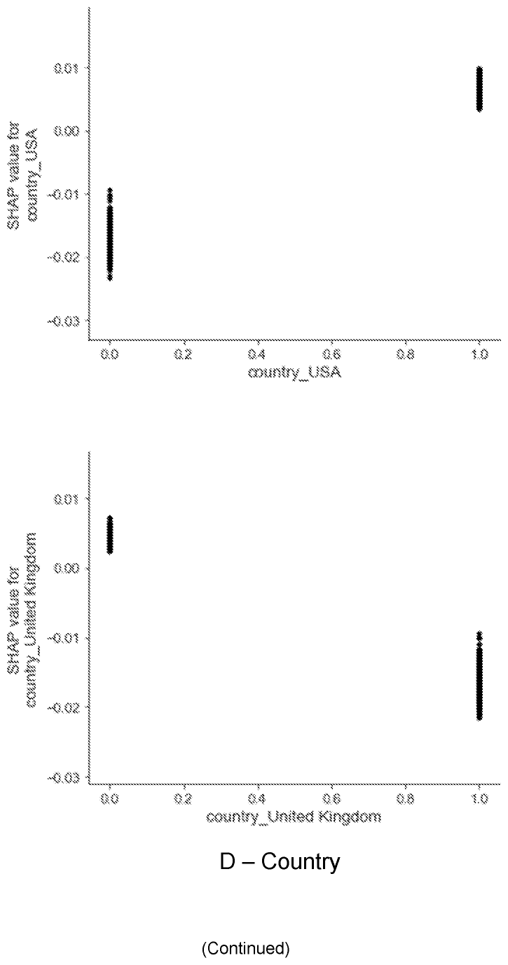

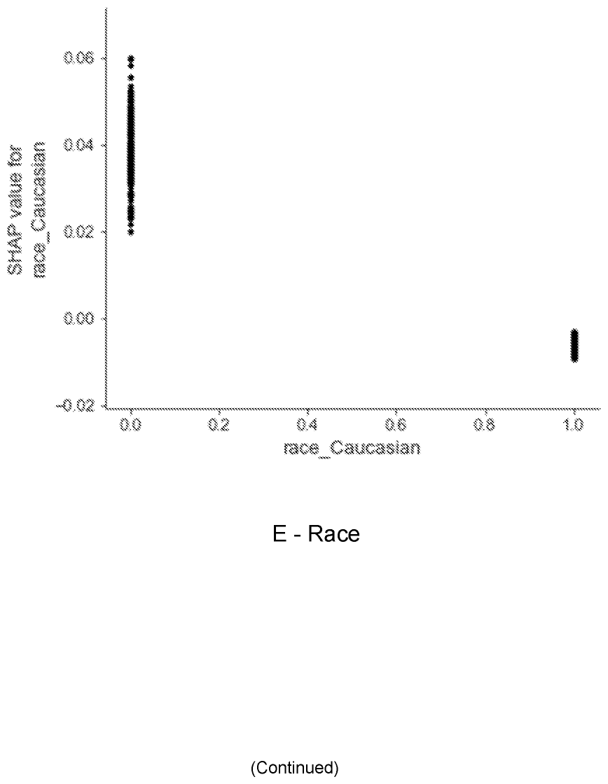

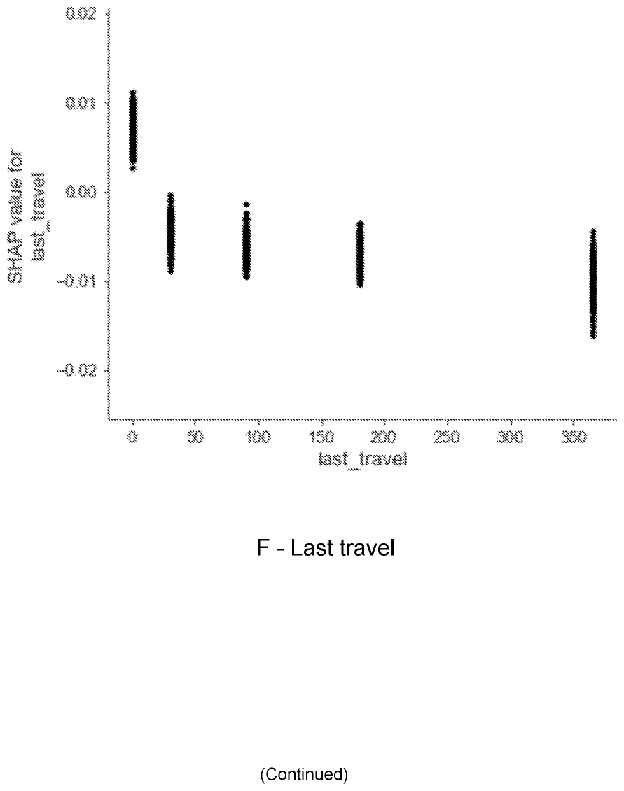

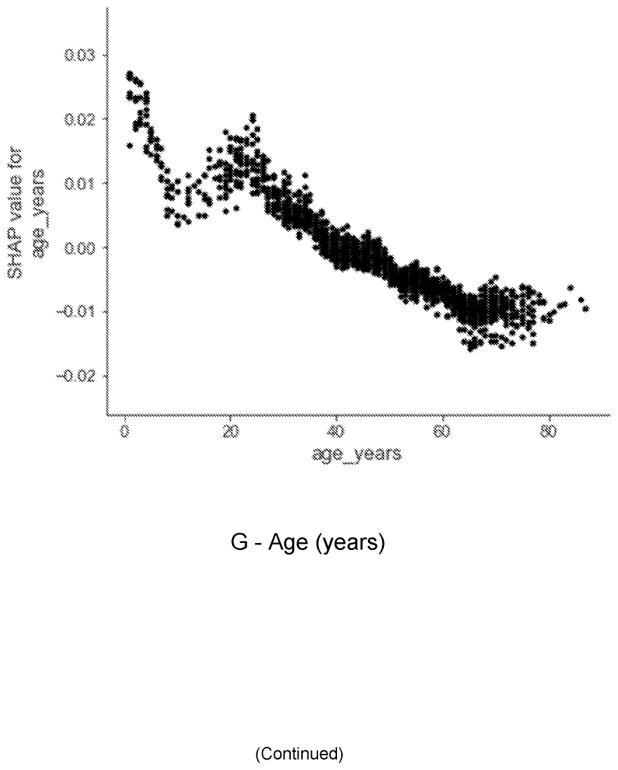

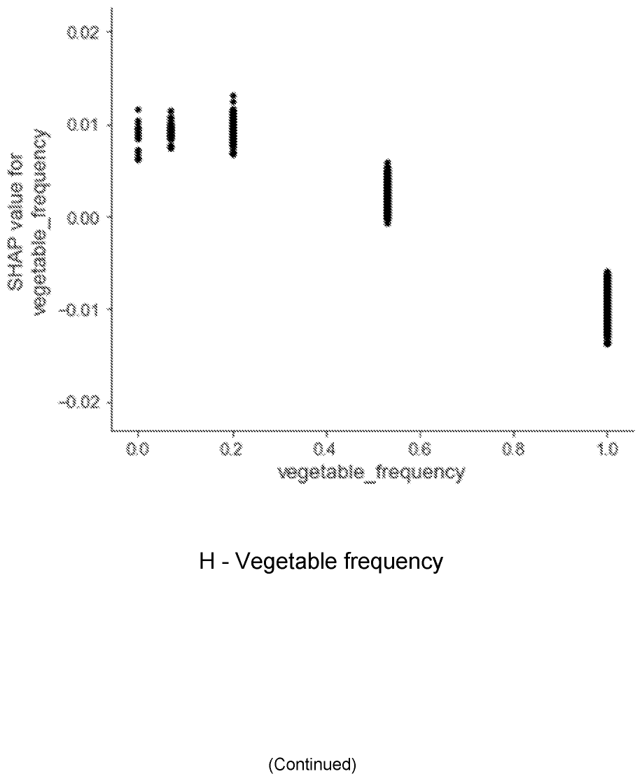

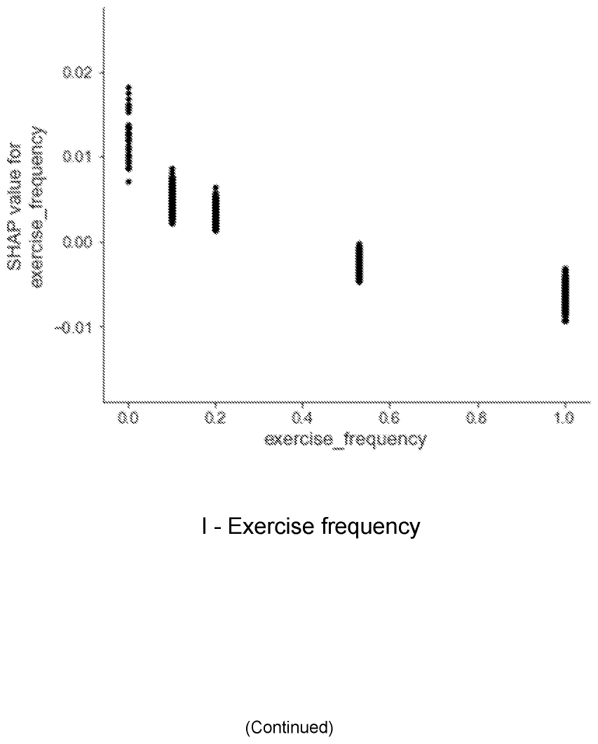

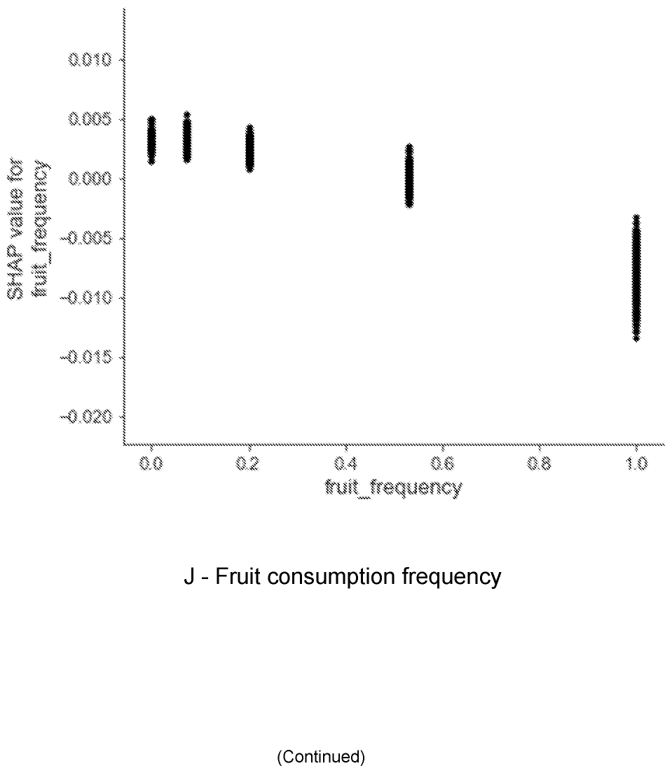

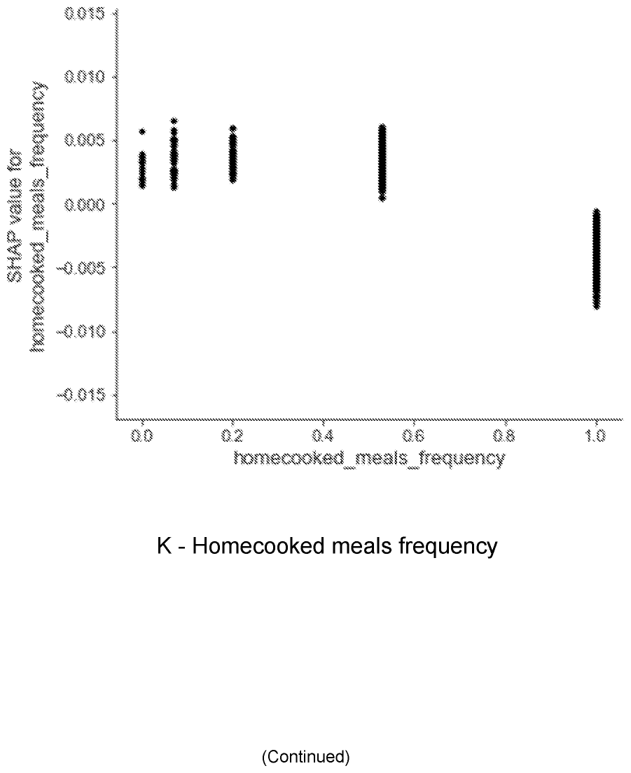

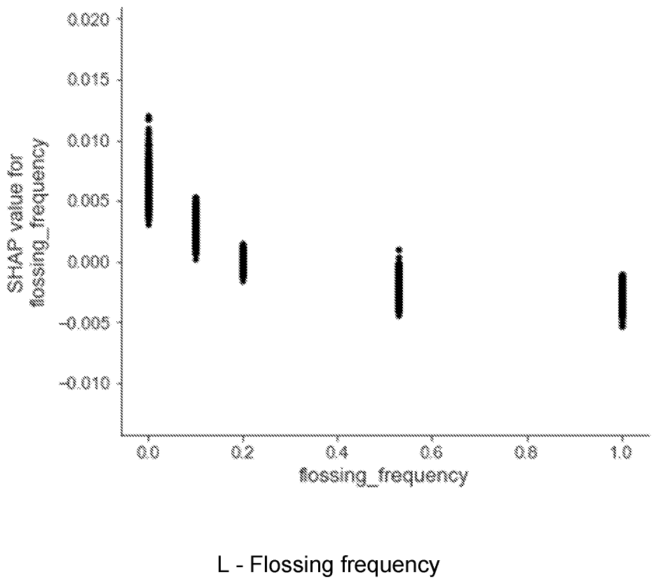

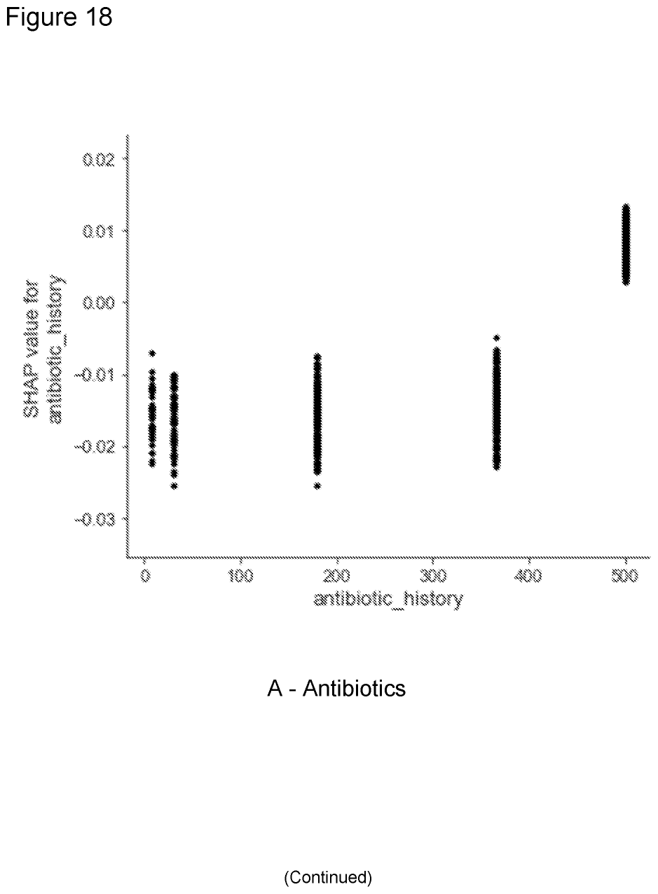

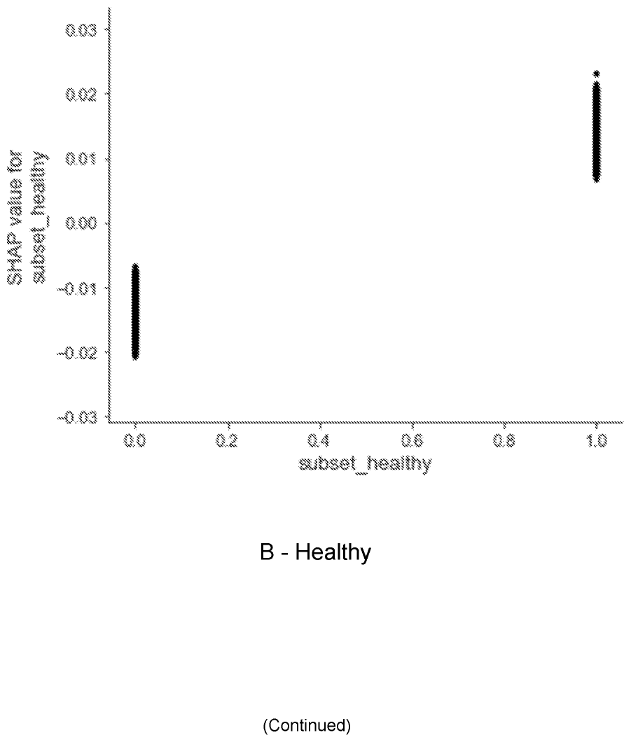

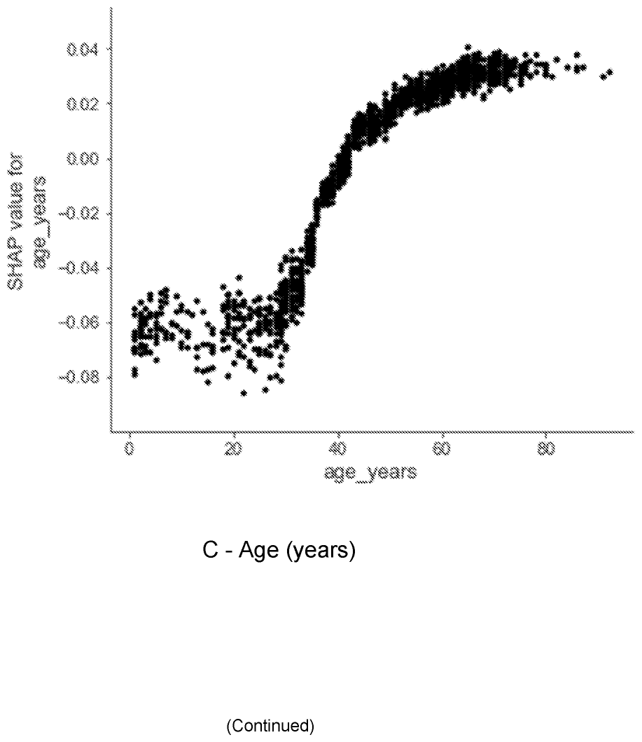

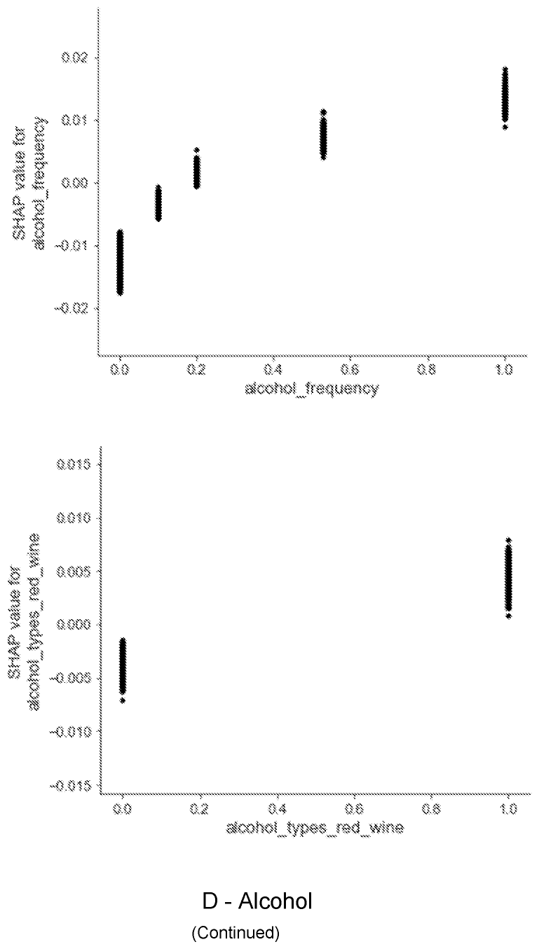

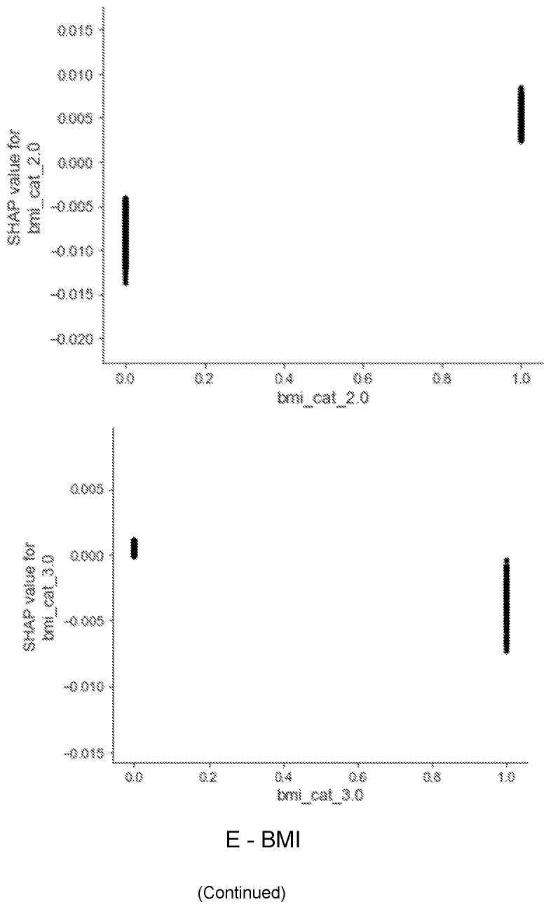

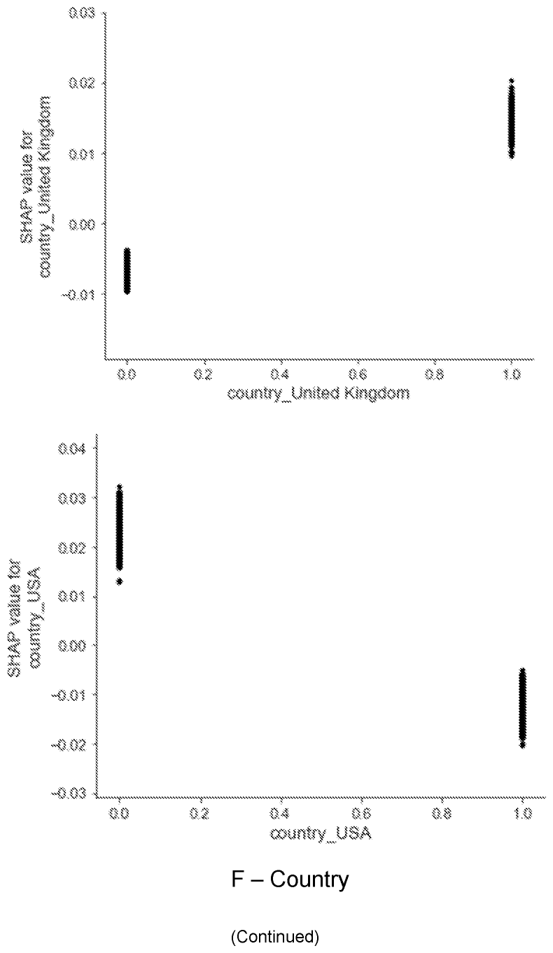

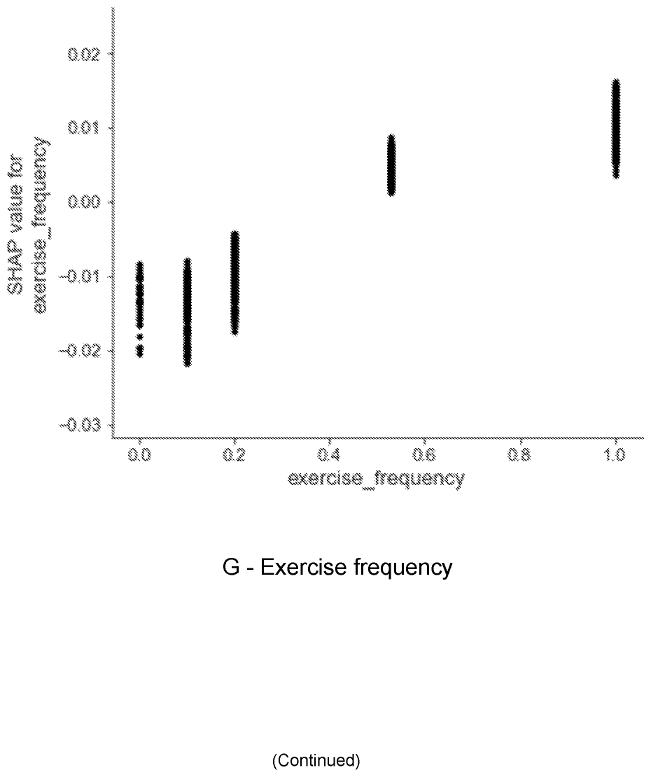

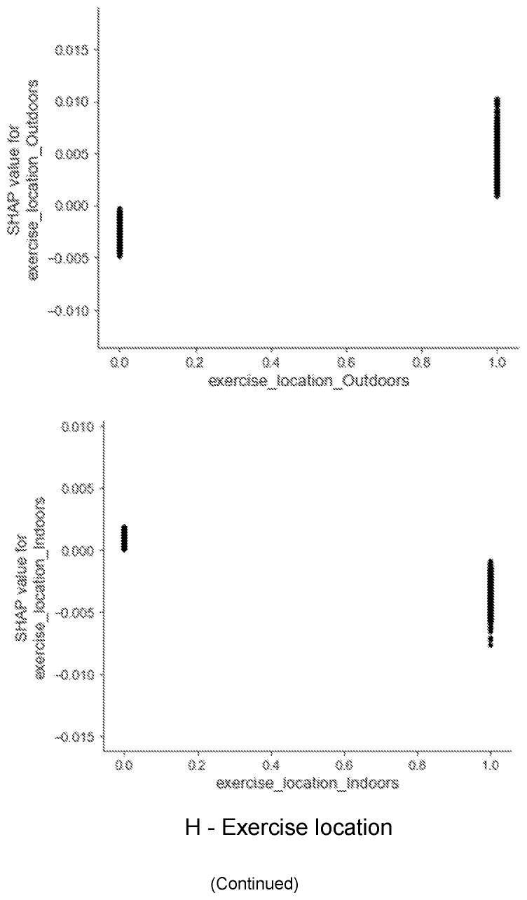

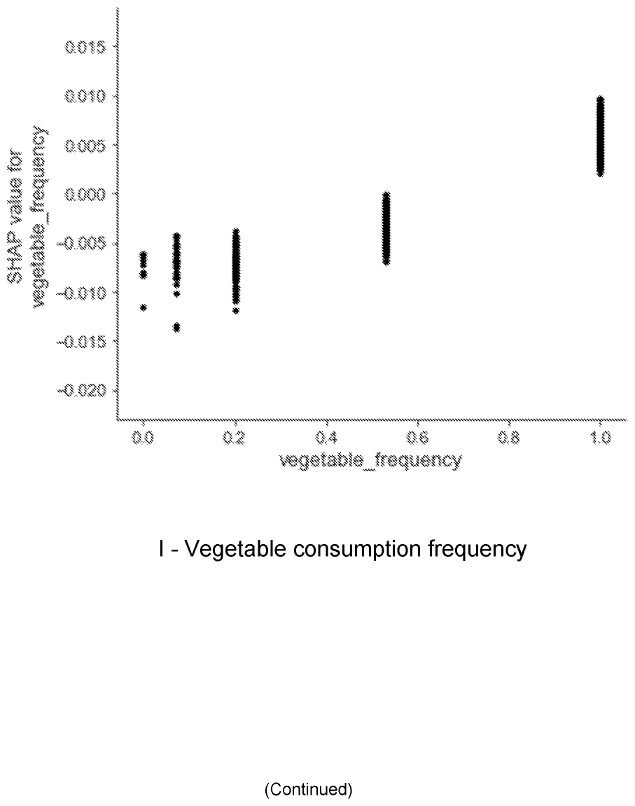

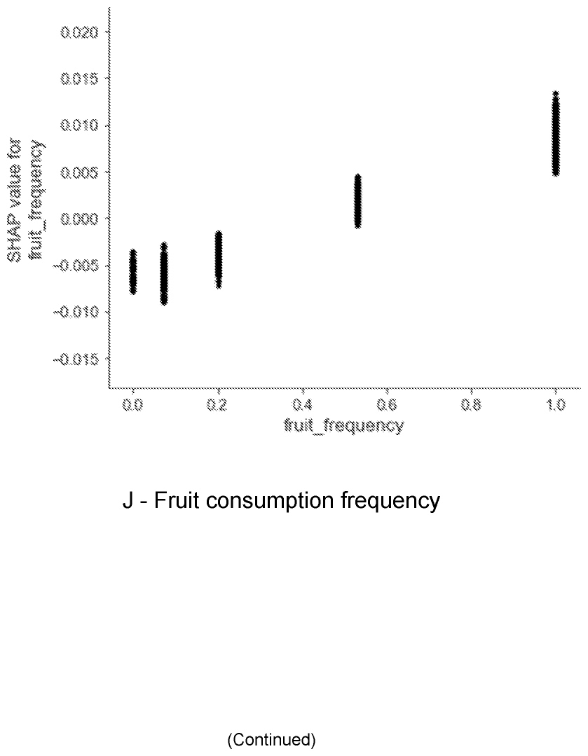

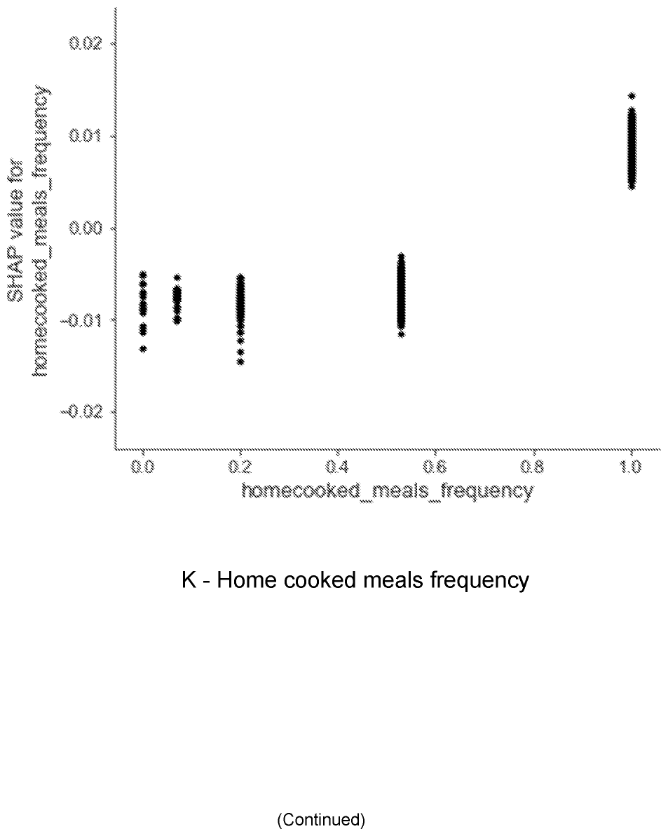

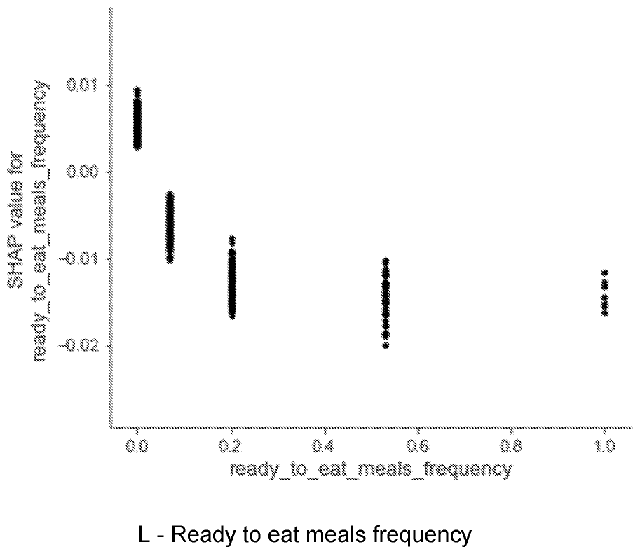

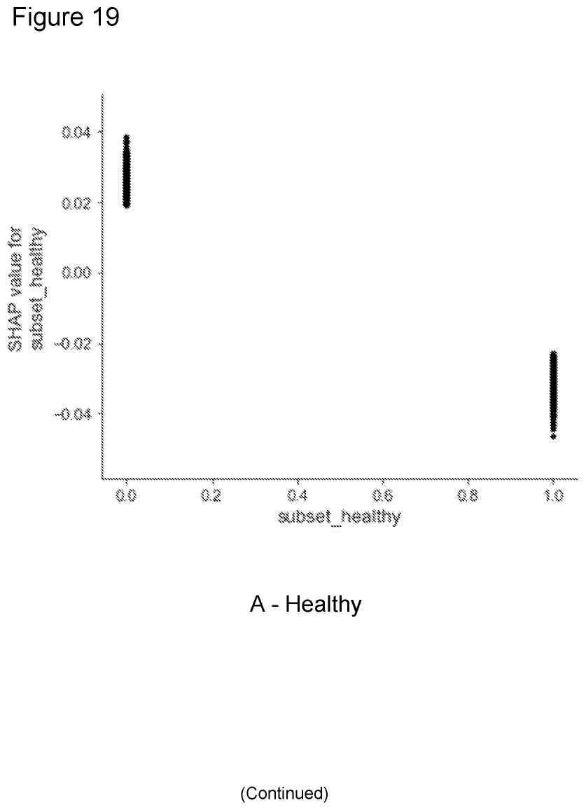

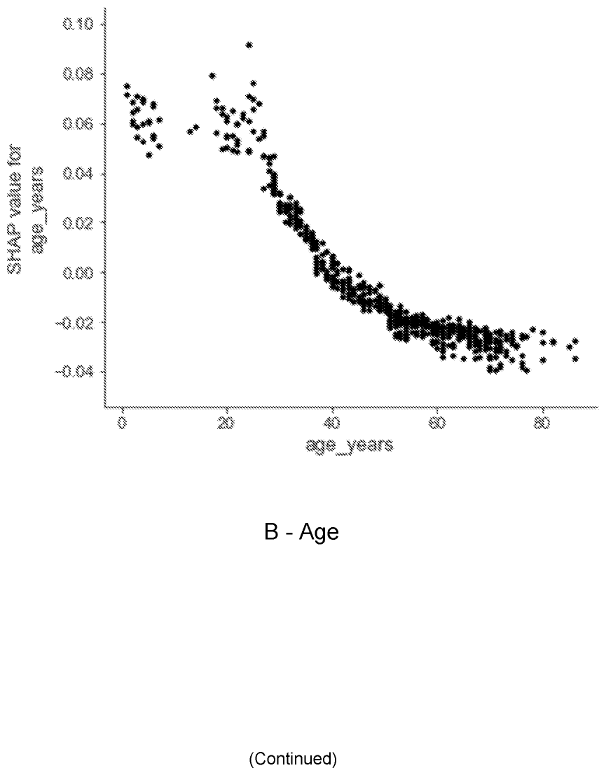

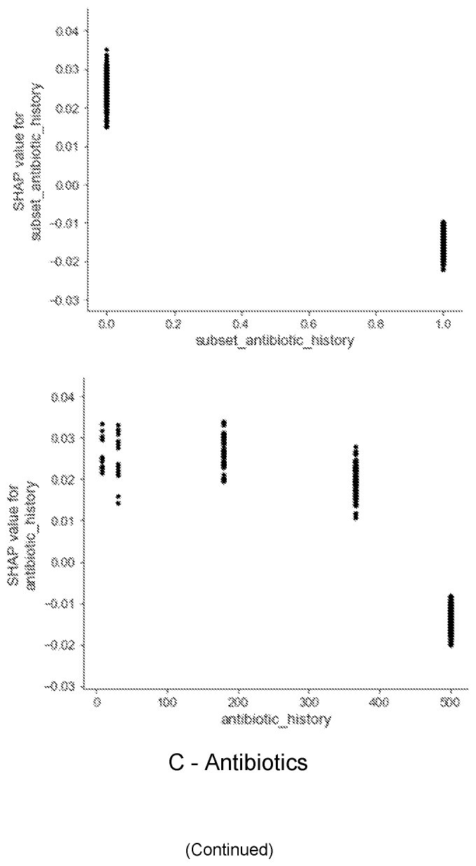

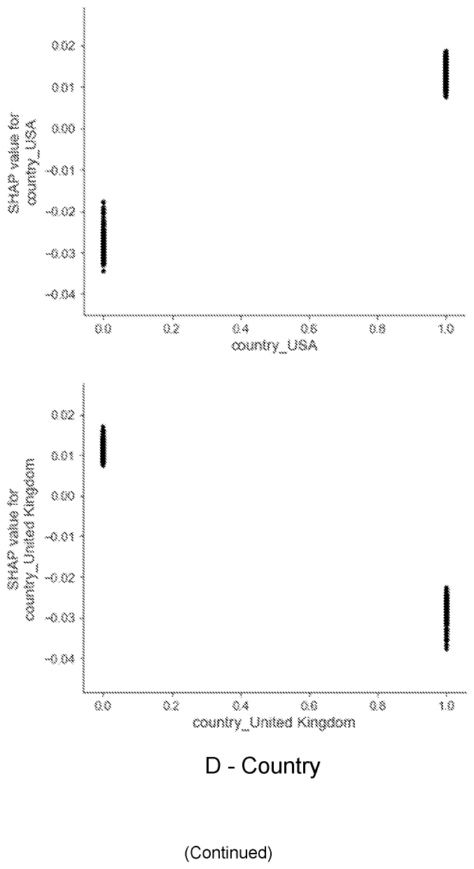

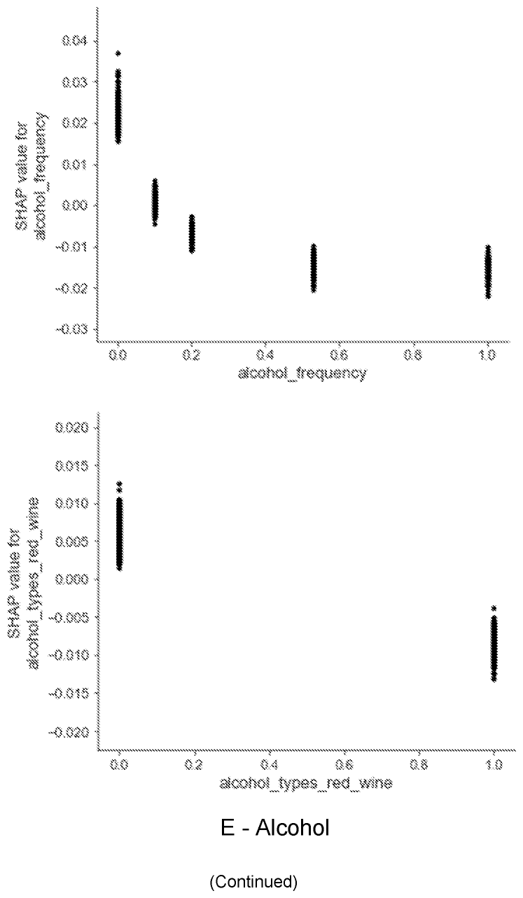

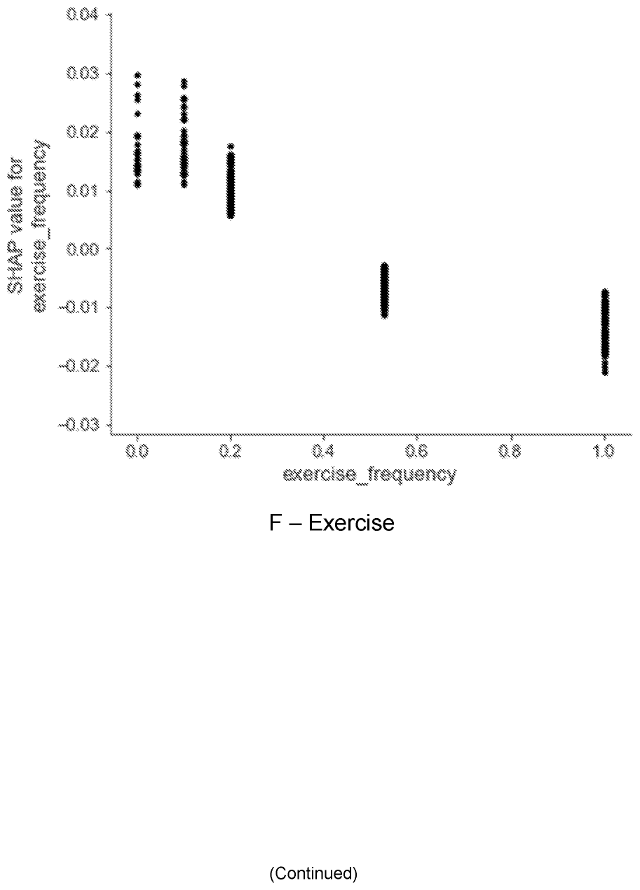

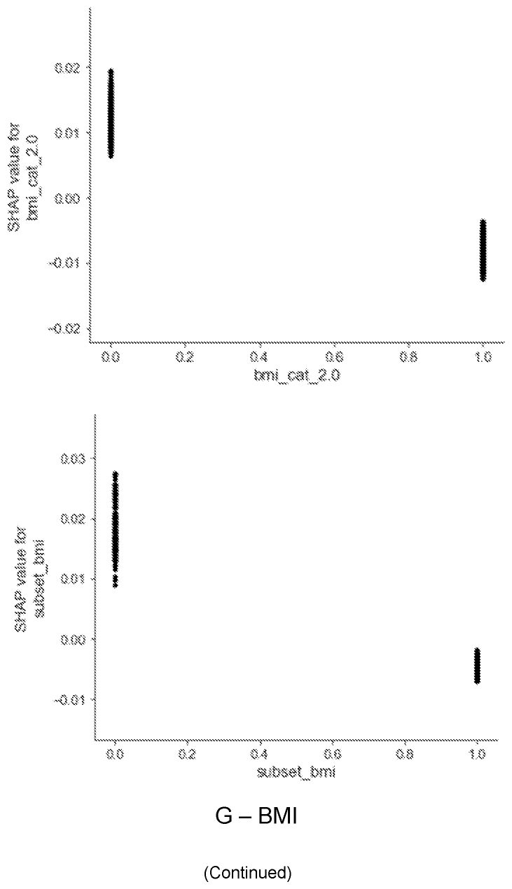

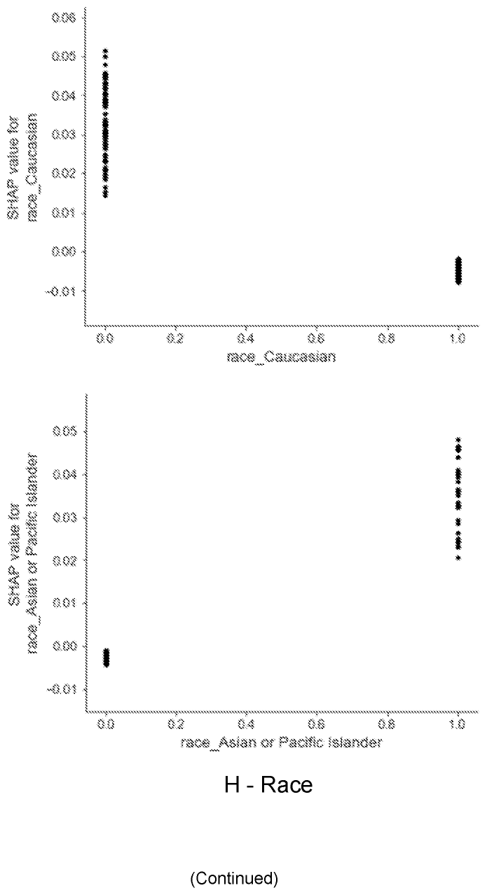

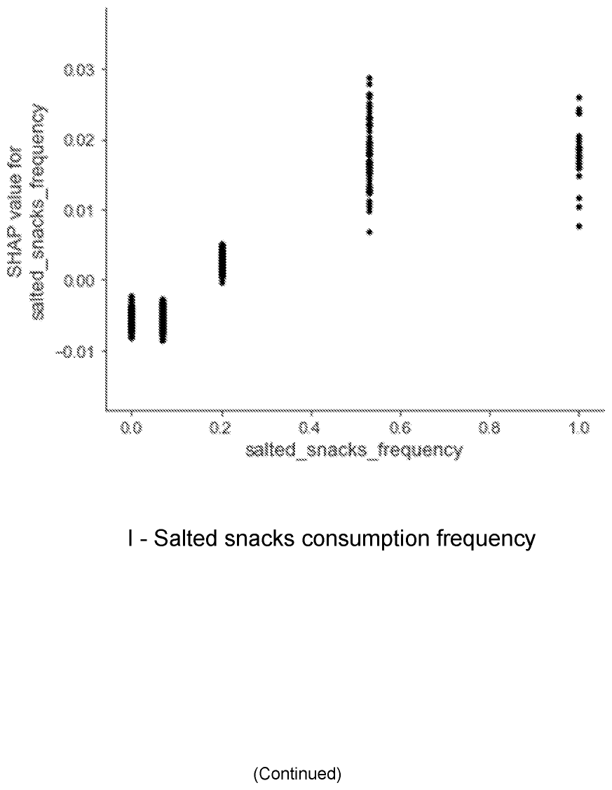

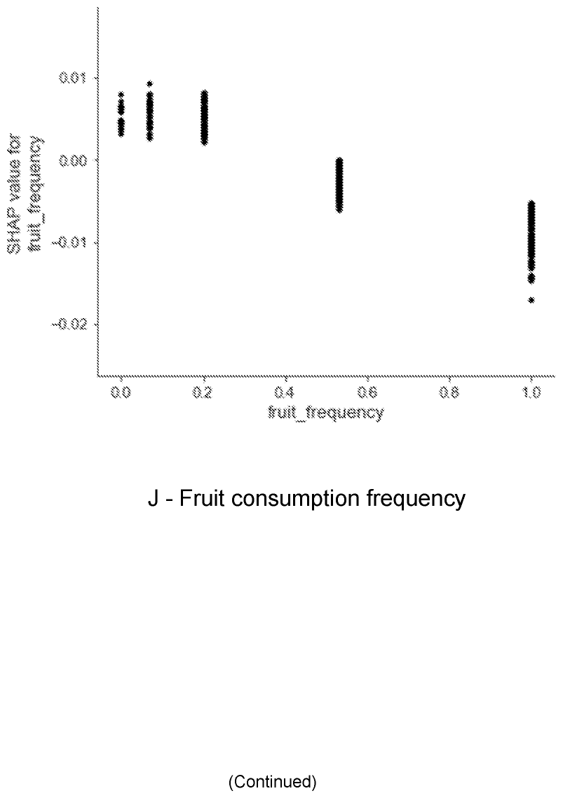

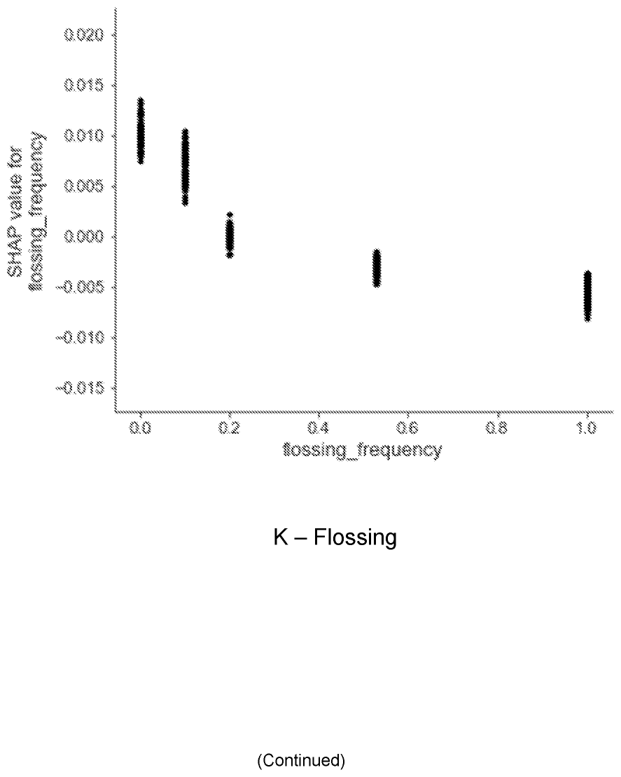

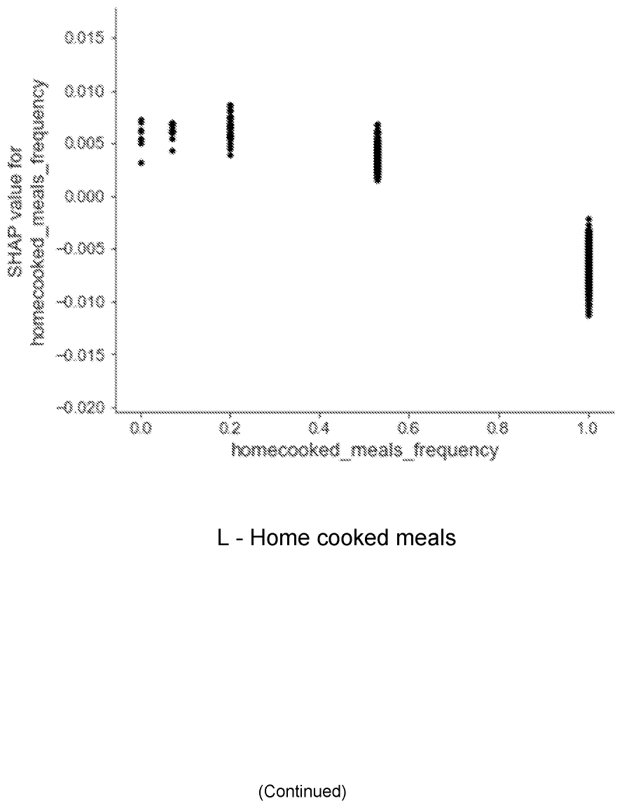

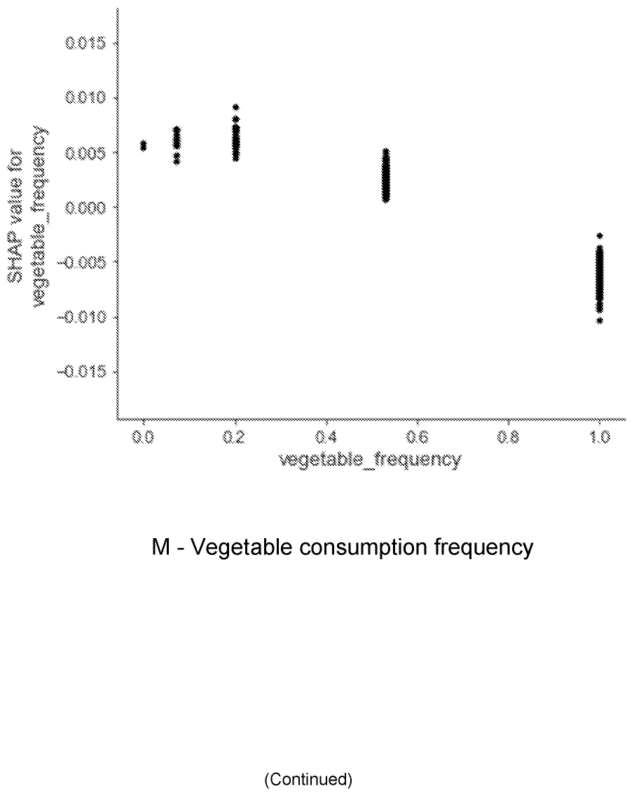

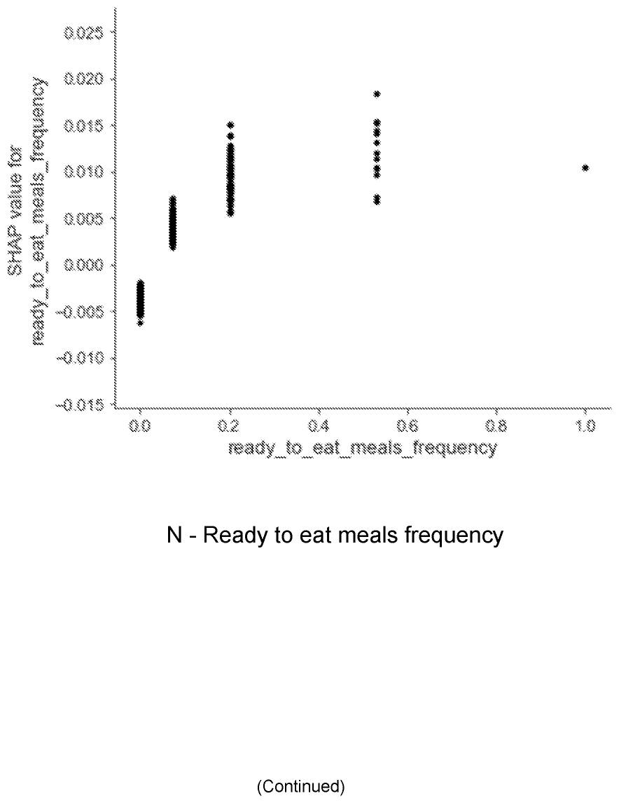

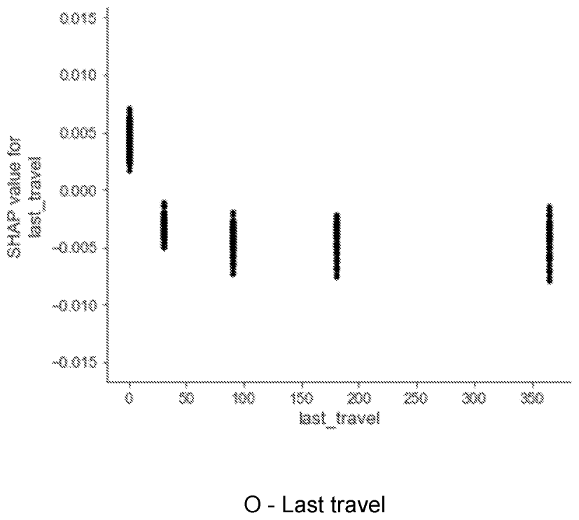

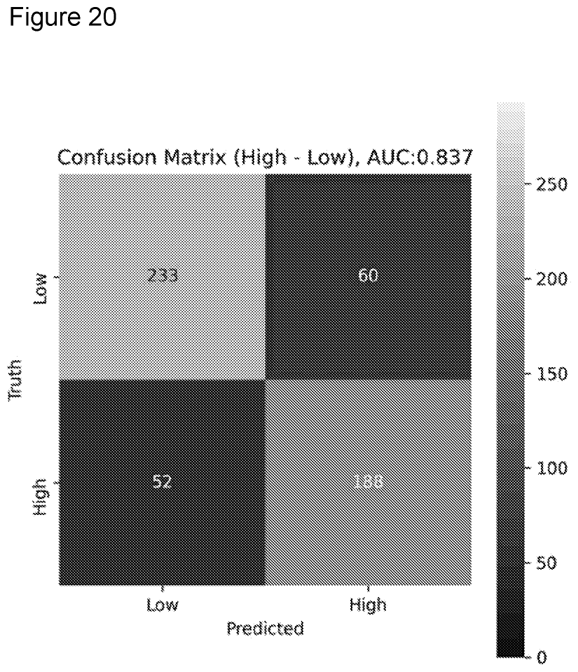

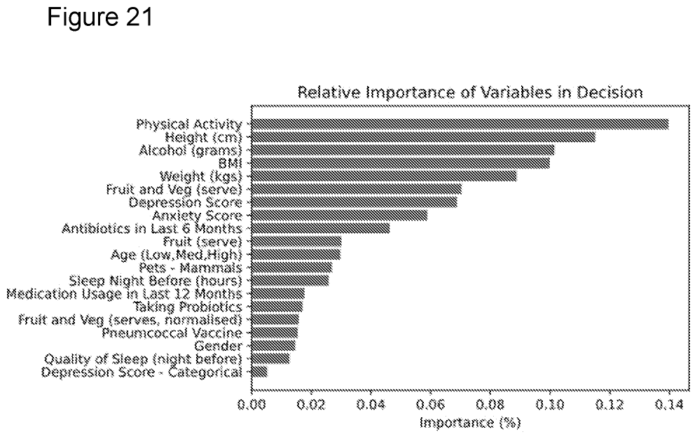

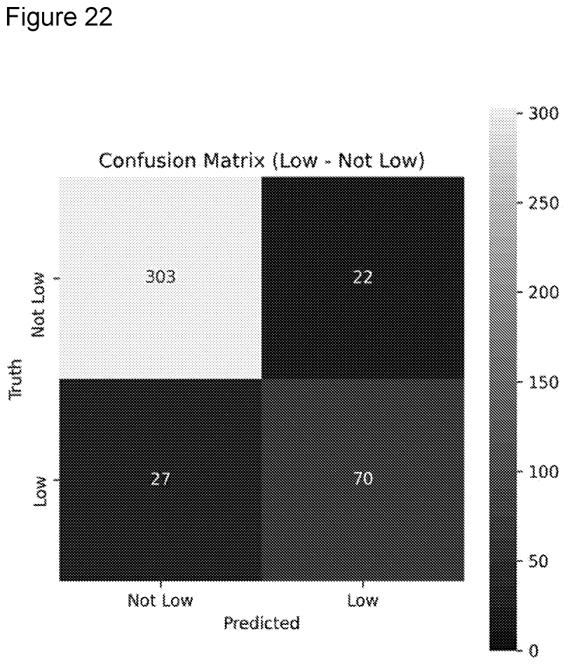

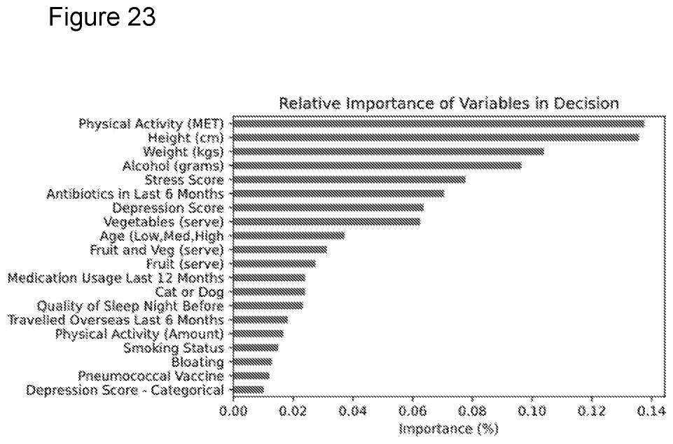

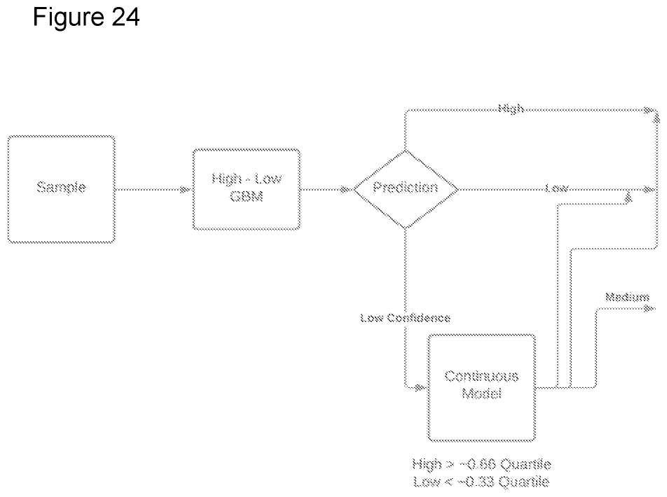

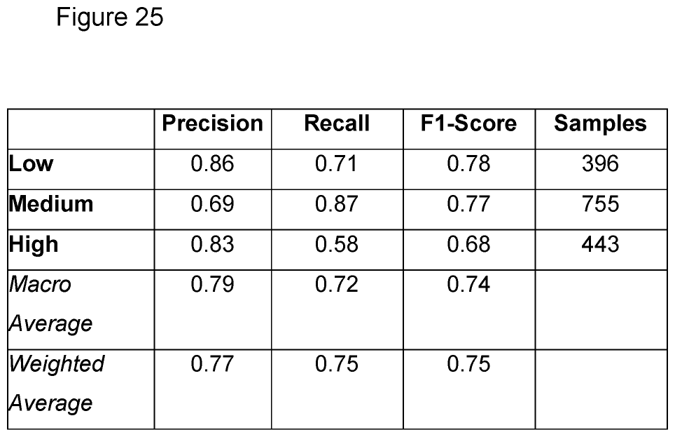

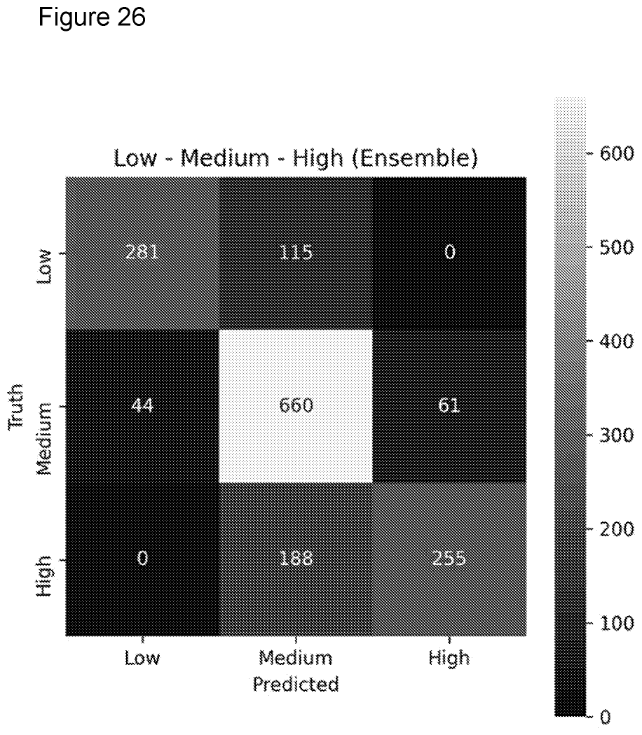

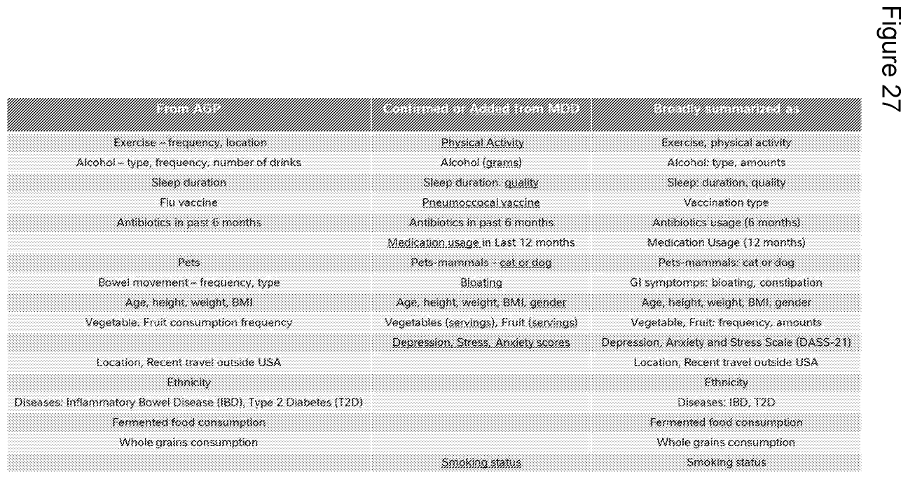

FIG. 6B—Feature importance plots for a Low versus notLow model

The feature importance plots for a Low versus notLow model on three diversity measures taken together with the definition of the bins based on mean and standard deviation and input data with those features with response-rate >0.85 The top 30 features for this model are shown in these plots. (i) shows the average impact per feature on the model output sorted in the order of importance from high to low. (ii) shows in more details the impact of a feature on the model output. The color gradation from grey to black indicates low to high values for that feature. The vertical line at 0.00 defines the directionality of impact with respect to the reference class “Low”—to the left is negative impact and to the right is positive impact on the model output.

FIG. 7A—Feature importance plots for a High versus notHigh model

The feature importance plots for a High versus notHigh model on three diversity measures taken together with the definition of the bins based on quartiles and input data with those features with response-rate >0.65 The top 30 features for this model are shown in these plots. (i) shows the average impact per feature on the model output sorted in the order of importance from high to low. (ii) shows in more details the impact of a feature on the model output. The color gradation from grey to black indicates low to high values for that feature. The vertical line at 0.00 defines the directionality of impact with respect to the reference class “High”—to the left is negative impact and to the right is positive impact on the model output.

FIG. 7B—Feature importance plots for a High versus notHigh model

The feature importance plots for a Low versus notLow model on three diversity measures taken together with the definition of the bins based on mean and standard deviation and input data with those features with response-rate >0.85 The top 30 features for this model are shown in these plots. (i) shows the average impact per feature on the model output sorted in the order of importance from high to low. (ii) shows in more details the impact of a feature on the model output. The color gradation from grey to black indicates low to high values for that feature. The vertical line at 0.00 defines the directionality of impact with respect to the reference class “notHigh”—to the left is negative impact and to the right is positive impact on the model output.

FIG. 8A—Feature importance plots for a Low versus High model

The feature importance plots for a Low versus High model on three diversity measures taken together with the definition of the bins based on quartiles and input data with those features with response-rate >0.65 The top 30 features for this model are shown in these plots. (i) shows the average impact per feature on the model output sorted in the order of importance from high to low. (ii) shows in more details the impact of a feature on the model output. The color gradation from grey to black indicates low to high values for that feature. The vertical line at 0.00 defines the directionality of impact with respect to the reference class “Low”—to the left is negative impact and to the right is positive impact on the model output.

FIG. 8B—Feature importance plots for a Low versus High model

The feature importance plots for a Low versus High model on three diversity measures taken together with the definition of the bins based on mean and standard deviation and input data with those features with response-rate >0.85 The top 30 features for this model are shown in these plots. (i) shows the average impact per feature on the model output sorted in the order of importance from high to low. (ii) shows in more details the impact of a feature on the model output. The color gradation from grey to black indicates low to high values for that feature. The vertical line at 0.00 defines the directionality of impact with respect to the reference class “Low”—to the left is negative impact and to the right is positive impact on the model output.

FIG. 9—Deconstructed performance of a Low versus notLow model

This Low versus notLow model was defined on three diversity measures taken together with the definition of the bins based on mean and standard deviation and input data with those features with response-rate >0.85 The figure shows the improvement in the performance of the model (AUC values of the ROC curves for Train (cross-validation) and holdout/Test set) as the features deemed important for this model by SHAP analysis were added one by one to be part of the model.

FIG. 10—Deconstructed performance of a High versus notHigh model

This High versus notHigh model was defined on three diversity measures taken together with the definition of the bins based on mean and standard deviation and input data with those features with response-rate >0.85 The figure shows the improvement in the performance of the model (AUC values of the ROC curves for Train (cross-validation) and holdout/Test set) as the features deemed important for this model by SHAP analysis were added one by one to be part of the model.

FIG. 11—Deconstructed performance of a Low versus High model

This Low versus High model was defined on three diversity measures taken together with the definition of the bins based on mean and standard deviation and input data with those features with response-rate >0.85 The figure shows the improvement in the performance of the model (AUC values of the ROC curves for Train (cross-validation) and holdout/Test set) as the features deemed important for this model by SHAP analysis were added one by one to be part of the model.

FIG. 12—Sample computer user interface for questionnaire

An example of an individual user's information in response to a set of questionnaires.

FIG. 13—Display of results of predicted microbiome status

An example of display of prediction results.

FIG. 14—SHAP force plot for an individual.

Based on the user's responses, it shows the features which positively or negatively affect the prediction of his microbiome status. The net result of these factors is the final prediction of the microbiome status for the individual.

FIG. 15—Personalized recommendations and advice

Examples of recommendations and advise to maintain or improve the microbiome status.

FIG. 16—Questionnaire

An example questionnaire needed for the models.

FIG. 17—SHAP dependence plots for key features in a Low versus notLow model

The SHAP dependence plots, for a Low versus notLow model on three diversity measures taken together with the definition of the bins based on quartiles and input data with those features with response-rate >0.65, are shown for key example features. The reference class here was “Low”, so the positive coefficients of SHAP value for the corresponding x-values of the feature indicate how much the model was affected by this feature in predicting the “Low” class.

FIG. 18—SHAP dependence plots for key features in a High versus notHigh model

The SHAP dependence plots, for a High versus notHigh model on three diversity measures taken together with the definition of the bins based on quartiles and input data with those features with response-rate >0.65, are shown for key example features. The reference class here was “High”, so the positive coefficients of SHAP value for the corresponding x-values of the feature indicate how much the model was affected by this feature in predicting the “High” class.

FIG. 19—SHAP dependence plots for key features in a Low versus High model

The SHAP dependence plots, for a Low versus High model on three diversity measures taken together with the definition of the bins based on quartiles and input data with those features with response-rate >0.65, are shown for key example features. The reference class here was “Low”, so the positive coefficients of SHAP value for the corresponding x-values of the feature indicate how much the model was affected by this feature in predicting the “Low” class.

FIG. 20—Summary of the classification results for the High-Low model

The Confusion Matrix obtained for the best performing model for the “High vs. Low” classification task is shown. This model used 20 metadata features, which were a mix of binary, categorical and continuous.

FIG. 21—The relative importance of the 20 metadata features used in the High-Low model.

The best performing model for the “High vs. Low” classification task used 20 metadata features, which were a mix of binary, categorical and continuous. The relative weighting of each feature for the model is shown here, with the most important feature at the top and so on.

FIG. 22—Summary of the classification results for the Low-notLow model.

The Confusion Matrix obtained for the best performing model for the “Low vs. notLow” classification task is shown.

FIG. 23—The relative importance of metadata features used in the Low-notLow model.

The relative weighting of the 20 features used by Low-notLow model is shown here, with the most important feature at the top and so on. Many of the 20 features (and their relative importance) overlapped with those determined to be optimal for the High-Low model, such as physical activity, height, weight, and alcohol consumption. Non-overlapping features in the Low-notLow model included stress, vegetable serves, cat or dog ownership, overseas travel, smoking, and bloating.

FIG. 24—Architecture for ensemble modelling.

Schema used for ensemble modelling (also known as stacked modelling) to predict three categories of diversity—Low, Medium, High. A binary classifier for “High vs. Low” with a continuous-output model using thresholds performed the best.

FIG. 25—Ensemble modelling results.

Performance results are shown for the “Low-Medium-High” modeling task using an ensemble model approach.

FIG. 26—Summary of the classification results for the ensemble model.

The Confusion Matrix obtained for “Low-Medium-High” classification of diversity is shown.

FIG. 27—Summarized key results

A consolidated set of features combining the main results obtained from AGP and MDD datasets is shown.

### DETAILED DESCRIPTION OF THE INVENTION

### Definitions

The “gut microbiota” is the composition of microorganisms (including bacteria, archaea and fungi) that live in the digestive tract.

The term “gut microbiome” may encompass both the “gut microbiota” and their “theatre of activity”, which may include their structural elements (nucleic acids, proteins, lipids, polysaccharides), metabolites (signaling molecules, toxins, organic, and inorganic molecules), and molecules produced by coexisting hosts and structured by the surrounding environmental conditions (see e.g. Berg, G., et al., 2020. Microbiome, 8(1), pp. 1-22).

In the present invention, the term “gut microbiome” may therefore be used interchangeably with the term “gut microbiota”.

“Microbiome-status” can be evaluated by several different measurements including determining the alpha diversity of bacteria found in the intestine.

“Alpha diversity of bacteria found in the intestine” summarizes the structure of an ecological community with respect to its “richness” (number of taxonomic groups), “evenness” (distribution of abundances of the groups), or both. In microbial ecology in the intestine, analyzing the alpha diversity of amplicon sequencing data is a common first approach to assessing differences between environments. In general, improving or maintaining the alpha diversity of microbial species in the intestine is an indication of a healthy microbiome.

“Operational taxonomic unit” (OTU) is an operational definition used to classify groups of closely related individuals. The term “OTU” also refers to clusters of organisms, grouped by DNA sequence similarity of a specific taxonomic marker gene (molecular OTU). OTUs are pragmatic proxies for “species” (microbial or metazoan) at different taxonomic levels, in the absence of traditional systems of biological classification as are available for macroscopic organisms. For several years, OTUs have been the most commonly used units of diversity, especially when analyzing small subunit 16S (for prokaryotes) or 18S rRNA (for eukaryotes) marker gene sequence datasets.

“Faith Phylogenetic Diversity” (Faith PD) is the most commonly used phylogenetic index. Faith PD is the phylogenetic analogue of taxon richness and is expressed as the number of tree units which are found in a sample. Reduced microbial PD in the human body may indicate reduced resilience, associated with many human diseases.

“Shannon index” is a measure of diversity, not richness. It measures the number of OTUs in sample (richness) but scales them based on the evenness of the community. For example, if the controls have more OTUs but a small number of those OTUs dominate the sample they will report a lower Shannon diversity than a community with fewer OTUs evenly distributed.

In the present invention, the “subject” may be a mammal, in particular a human. The human may be a male and/or a female human. For example, the human may be an adult, for example, an adult that is 18 years old or older. Further, for example, the adult may be 30 years old or older, 40 years old or older, or 50 years old or older. According to one embodiment of the present invention, the adult may be from 18-99 years old, preferably from 20-70 years old. The mammal may be a pet animal, in particular a dog or a cat.

In several embodiments, the systems and methods of the invention contribute to microbiome-status assessment of a subject by providing different methods to estimate this such as predicting if the microbiome diversity is “Low” or “notLow”, “High” or “notHigh”, or “Low”, “Medium”, “High” with respect to the microbiome distribution seen for a normal population for the parameters of the alpha diversity of bacteria in the intestine.

In some embodiments, Low, notLow alpha-diversity groups are defined as “Low” as being below the first or lower quartile of the population OTU distribution and “notLow” as the rest of the distribution.

In some embodiments, Low, notLow alpha-diversity groups are defined as “Low” as being below the first or lower quartile of the population FAITH PD distribution and “notLow” as the rest of the distribution.

In some embodiments, Low, notLow alpha-diversity groups are defined as “Low” as being below the first or lower quartile of the population SHANNON distribution and “notLow” as the rest of the distribution.

In some embodiments, Low, notLow alpha-diversity groups are defined as: “Low” as being below the first or lower quartile on these three distributions of OBSERVEDOTU, FAITHPD and SHANNON and “notLow” as on these three together OBSERVEDOTU, FAITHPD and SHANNON for the rest of the distribution.

In some embodiments, High, notHigh alpha-diversity groups are defined as “High” being above the third or upper quartile of the population OBSERVEDOTU distribution and “notHigh” as the rest of the distribution.

In some embodiments, High, notHigh alpha-diversity groups are defined as “High” being above the third or upper quartile of the population FAITHPD distribution and “notHigh” as the rest of the distribution.

In some embodiments, High, notHigh alpha-diversity groups are defined as “High” being above the third or upper quartile of the population SHANNON distribution and “notHigh” as the rest of the distribution.

In some embodiments, High, notHigh alpha-diversity groups are defined as: “High” as being above the third or upper quartile on these three distributions of OBSERVEDOTU, FAITHPD and SHANNON and “notHigh” as on these three together OBSERVEDOTU, FAITHPD and SHANNON for the rest of the distribution.

In some embodiments, Low, notLow, High, notHigh alpha-diversity groups are defined as in different population data sets with different numerical cut-offs that would be apparent to a person skilled in the art.

In some embodiments, Low, High alpha-diversity groups are defined as: “Low” being below the first or lower quartile on the OBSERVEDOTU distribution and “High” being above the third or upper quartile on OTU distribution.

In some embodiments, Low, High alpha-diversity groups are defined as: “Low” being below the first or lower quartile on the FAITHPD distribution and “High” being above the third or upper quartile on FAITHPD distribution.

In some embodiments, Low, High alpha-diversity groups are defined as: “Low” being below the first or lower quartile on the SHANNON distribution and “High” being above the third or upper quartile on SHANNON distribution.

In some embodiments, Low, High alpha-diversity groups may be defined with different numerical cut-offs that would be apparent to a person skilled in the art.

In some embodiments, on any of the diversity measures, “Low” alpha-diversity group is defined as the data which is less than the mean minus the standard deviation and “notLow” alpha-diversity group as the rest of the data.

In some embodiments, on any of the diversity measures, “Low” alpha-diversity group is defined as the data which is less than first or lower quartile minus the inter-quartile range and “notLow” alpha-diversity group as the rest of the data.

In some embodiments, on any of the diversity measures, “Low” alpha-diversity group is defined as the data which is less than first or lower quartile minus 1.5×inter-quartile range and “notLow” alpha-diversity group as the rest of the data.

In some embodiments, on any of the diversity measures, “High” alpha-diversity group is defined as the data which is more than the mean plus the standard deviation and “notLow” alpha-diversity group as the rest of the data.

In some embodiments, on any of the diversity measures, “High” alpha-diversity group is defined as the data which is more than first or lower quartile plus the inter-quartile range and “notHigh” alpha-diversity group as the rest of the data.

In some embodiments, on any of the diversity measures, “Low” alpha-diversity group is defined as the data which is less than the mean minus the standard deviation and “High” alpha-diversity group as the data which is more than the mean plus the standard deviation.

In some embodiments, on any of the diversity measures, “Low” alpha-diversity group is defined as the data which is less than the first or lower quartile minus the inter-quartile range and “High” alpha-diversity group as the data which is more than the first lower quartile plus the inter-quartile range

In some embodiments, the groups have been defined in the following manner on any of the alpha-diversity measures, singly or taken together:
for Low vs. not Low: <=10% or <=15% or <=20% or <=25% or <=30% of data in Low vs. rest >10% or >15% or >20% or >25% or >30% of data in notLow, respectively
for High vs. notHigh: >=90% or >=85% or >=80% or >=75% or >=70% of data in High vs rest <90% or <85% or <80% or <75% or <70% of data in notHigh, respectively
for Low vs. High: <=10% in Low vs. >=90% in High or <=15% in Low vs >=85% in High or <=20% in Low vs >=80% in High or <=25% in Low vs >=75% in High or <=30% in Low vs >=70% in High.

In some embodiments, both measures of alpha diversity are used to categorise microbiome profiles as having one of the following levels of diversity: Low, -notLow, Medium, notHigh and High, as appropriate for defining the population-stratified groups.

Here, SD=Shannon diversity, SR=species richness, with Natural logarithm base for Shannon index.

In some embodiments, High and Low bins are defined as Low=SD+SR is in the lowest tercile of samples (0.33) and High=SD+SR is in the highest tercile of samples (0.66). Thus, threshold is for both measures, that is less than third tercile for BOTH. Middle values are discarded.

In some embodiments, Low and Not Low bins are defined as Low=SD+SR is in the lowest quartile of samples 0.25) and Not Low=SD+SR is in the upper three quartiles of samples (>0.25). Thus, both measures <=0.25 is Low, else Not Low.

In some embodiments, Low, Medium, High bins are defined as based on terciles. Low=SD+SR in lowest tercile (≤0.33), Medium=SD+SR in middle tercile (0.34-0.65), and High=SD+SR in upper tercile (≥0.66). Thus, the threshold for both measures, that is less than third tercile for BOTH. Everything else is assigned as Medium.

It should be appreciated that the groups can be defined in many other possible ways which are variations of the above but with somewhat different definitions such as median/mean+/−1 standard deviation or median/mean+/−½ standard deviation or median/mean+/−½ inter-quartile range or a different % of data points going into the groups than what has been mentioned above which is apparent to a person skilled in the art of data analytics.

“Receiver Operating Characteristic” (ROC) curve is one of the best-developed statistical tools for describing the performance of” diagnostic tests measured on continuous scale. ROC use is based on having two outcomes from the prediction. Numerical indices of the ROC curves were used to summarize the curves. These summary measures were also used for comparing the ROC curves.

“Area Under the ROC curve” (AUC) is the most widely used summary measure. A perfect prediction model with the ideal ROC curve has the value AUC=1.0, while random prediction model has AUC=0.5. ROC curve AUC value moving from 0.5 towards 1.0 indicates improving and better performance of prediction models.

Many other measures of model performance can be calculated on the confusion matrix, such as true positives (TP), false positives (FP), true negatives (TN), false negatives (FN), total predicted positive, total predicted negative, total actual positive, total actual negative, sensitivity/hit rate/recall/true positive rate (TPR), specificity/selectivity/true negative rate (TNR), prevalence, precision/positive predictive value (PPV), negative predictive value (NPV), miss rate/false negative rate (FNR), fall-out/false positive rate (FPR), false discovery rate (FDR), false omission rate (FOR), prevalence threshold (PT), threat score (TS)/critical success index (CSI), accuracy (ACC), balanced accuracy (BA), random accuracy, total accuracy, F1 score, Matthews correlation coefficient (MCC), Fowlkes Mallows index (FM), Informedness/Bookmaker informedness (BM), Markedness (MK)/deltaP, Positive likelihood ratio (LR+), Negative likelihood ratio (LR−), Diagnostic odds ratio (DOR), Kappa.

AUC-ROC is the area under the curve which is created by plotting the true positive rate against the false positive rate at various probabilities. AUC-PR is the area under the precision recall curve.

Various embodiments of the disclosed system satisfy the general goal, given a questionnaire, to assess the overall microbiome status of the individual. The microbiome status assessment depends on the general characteristics of the individual (for ex., gender, age, weight, body measurements, physical activity level, and other health-related conditions like IBS or diabetes etc.) and the subsequent recommendations to maintain or advise to improve the microbiome-health likewise incorporate characteristics of the individual as gleaned from his responses to the sets of questionnaire.

In various embodiments, the system disclosed herein calculates from the various responses obtained from the individual's answering of the set of questionnaires and displays the respective impact on the microbiome status. Since the overall prediction also depends on the general characteristics of the individual (BMI, age, weight, ethnicity, etc.), the recommendations to maintain or improve the microbiome likewise depend on characteristics of the individual. In these embodiments, the system determines one or more of these factors as being detrimental or beneficial for the individual's microbiome for whom the recommendations are being calculated. The disclosed system and methods in these embodiments recommend either reductions or additions to these modifiable factors that can be adapted for the individual.

In various embodiments, the disclosed system provides a recommendation or advisory function, wherein the system suggests combinations of features or factors that will result in an improved or optimal microbiome-status. For example, if a user accesses the system after antibiotics usage and indicates so in his responses to the set of questionnaires, the disclosed system may predict the microbiome status accordingly and indicate the reasoning behind the said predictions in terms of individual factors such as antibiotics usage influencing the final prediction. Thus, the system disclosed herein can operate not only as a prediction system, but also as a recommendation engine to provide personalized advice to help individuals reach their goal of having a good microbiome status.

The term “feature” is used repeatedly herein. In some embodiments, the term “feature” as used herein refers to the input parameters to the models. The term includes responses obtained from the sets of questionnaires. As used herein the term feature may include, for example, general information on anthropometric measures such as age, gender, height, weight; lifestyle characteristics such as exercise/physical activity, alcohol usage, smoking status, anxiety status, depression status, stress status; travel; medication use such as antibiotics; disease status such as IBD, diabetes and the like. These features are not necessarily mutually exclusive. For example, certain features such as age and exercise may be co-related or certain medications use may be associated with some diseases or certain diseases may exist together as co-morbidities.

As is known in the art, an anthropometric measure is a measurement of a subject. In one embodiment, the anthropometric measure is selected from the group consisting of gender, age (in years), weight (in kilograms), height (in meters), and body mass index (in kg/m-2). Other anthropometric measures will also be known to the skilled person in the art.

The term “ethnicity” or “race” may be used in the context of specific geographic populations to cluster different sub-population groups. For example, in the United States, the categories include: White, Black or African American, American Indian or Alaska Native, Asian, and Native Hawaiian or Other Pacific Islander.

By the term “lifestyle characteristic” is meant any lifestyle choice made by a subject, this includes all dietary intake data, activity measures or data from questionnaires of lifestyle, motivation or preferences. In one embodiment, the lifestyle characteristic is whether the subject is an alcohol drinker or a non-drinker. In another embodiment, the lifestyle characteristic is whether the subject is a smoker or a non-smoker. In another embodiment, the lifestyle characteristic is whether the subject is a regular exerciser or not.

By the term “anxiety status” is meant feeling of unease affecting the subject, such as worry or fear, that can be mild or severe. Anxiety is commonly tested via validated questionnaires. For example, The General Health Questionnaire consists of sixty questions about mild somatic and anxiety symptoms. Thirty- and 12-item questionnaires are also commonly used. The Patient Health Questionnaire (PHQ-9) and the Center for Epidemiologic Studies Depression Scale (CES-D) as further examples of anxiety scales/scores. In another embodiment, the anxiety score may be self-assessed by the subject.

In a preferred embodiment, the anxiety score is measured by the DASS21methodology (Lovibond, S. H. & Lovibond, P. F. (1995). Manual for the Depression Anxiety & Stress Scales. (2nd Ed.) Sydney: Psychology Foundation; Lovibond P: Overview of the DASS and Its Uses. Retrieved from http://www2.psy.unsw.edu.au/dass/over.htm). For MDD data, the DASS-21 questionnaire was used as given here (https://maic.qld.cov.au/wp-content/uploads/2016/07/DASS-21.pdf) with scores obtained on the DASS-21 multiplied by 2 to calculate the final score in order to obtain the recommended cut-off scores for conventional severity labels (normal, moderate, severe).

The DASS is a set of three self-report scales designed to measure the negative emotional states of depression, anxiety and stress. Each of the three DASS scales contains 14 items, divided into subscales of 2-5 items with similar content. The Depression scale assesses dysphoria, hopelessness, devaluation of life, self-deprecation, lack of interest/involvement, anhedonia, and inertia. The Anxiety scale assesses autonomic arousal, skeletal muscle effects, situational anxiety, and subjective experience of anxious affect. The Stress scale is sensitive to levels of chronic non-specific arousal. It assesses difficulty relaxing, nervous arousal, and being easily upset/agitated, irritable/over-reactive and impatient. Subjects are asked to use 4-point severity/frequency scales to rate the extent to which they have experienced each state over the past week. Scores for Depression, Anxiety and Stress are calculated by summing the scores for the relevant items. In addition to the basic 42-item questionnaire, a short version, the DASS-21, is available with 7 items per scale. Characteristics of high scorers on Anxiety scale by DASS-21 are apprehensive, panicky; trembly, shaky; aware of dryness of the mouth, breathing difficulties, pounding of the heart, sweatiness of the palms; worried about performance and possible loss of control.

By the term “depression status” is meant a mood disorder that causes a persistent feeling of sadness and loss of interest. Also called major depressive disorder or clinical depression, it affects how the subject feel, think and behave and can lead to a variety of emotional and physical problems. In an embodiment, the depression rating scale/score is completed by the subject. The Beck Depression Inventory, for example, is a 21-question self-report inventory that covers symptoms such as irritability, fatigue, weight loss, lack of interest in sex, and feelings of guilt, hopelessness or fear of being punished. In a preferred embodiment, the depression score is assessed through the DASS-21 method as mentioned in detail in the previous para. Characteristics of high scorers on Depression scale by DASS-21 are self-disparaging; dispirited, gloomy, blue; convinced that life has no meaning or value; pessimistic about the future; unable to experience enjoyment or satisfaction; unable to become interested or involved; an dslow, lacking in initiative.

Similarly, stress score is also preferably measured by the DASS-21 method as mentioned above. Characteristics of high scorers on Stress scale by DASS-21 are over-aroused, tense; unable to relax; touchy, easily upset; irritable; easily startled; nervy, jumpy, fidgety; and intolerant of interruption or delay.

In a particularly preferred embodiment, the user inputs into the device his responses to the questions, for example, on health status, medication usage, antibiotic usage, location, age (years), BMI, last travel, race, alcohol consumption (e.g. type, frequency, amounts of alcohol), smoking status, weight (kg), height (cm), IBD, GI symptoms (e.g. bloating, bowel movement quality), weight change, depression status, anxiety status, stress status, season, living with whom, drinking water source etc. The device then processes this information and provides a prediction on the user's microbiome status in terms of being “Low” or “notLow”, as per the definitions given above.

In a particularly preferred embodiment, the user inputs into the device his responses to the questions, for example, on age (years), location, health status, alcohol consumption (e.g. alcohol types (red or white wine, unspecified); alcohol frequency, alcohol amounts), smoking status, medication usage, antibiotic usage, weight (kg) last travel, anxiety status, depression status, stress status, chickenpox, vaccination status (e.g. flu vaccine date, pneumococcal vaccine date), lactose intolerance, race, cosmetics frequency etc. The device then processes this information and provides a prediction on the user's microbiome status in terms of being “High” or “notHigh”, as per the definitions given above.

In a particularly preferred embodiment, the user inputs into a computer implemented device the responses to the questions in a questionnaire, for example, on health status, age (years), location, medication usage, antibiotic usage, alcohol consumption (e.g. alcohol types (red or white wine, unspecified); alcohol frequency, alcohol amounts), smoking status, vaccination status, race, BMI, last travel, anxiety status, depression status, stress status, chickenpox, IBD, GI symptoms (e.g. bloating, bowel movement quality), weight (kg). The device then processes this information and provides a prediction on the user's microbiome status in terms of being “Low” or “High”, as per the definitions given above. Examples of suitable questionnaires are found in FIG. 12 or FIG. 16.

The device may generally be a server on a network. However, any device may be used if it can process data such as biomarker data and/or anthropometric and lifestyle data using a processor, a central processing unit (CPU) or the like. The device may, for example, be a smartphone, a tablet terminal or a personal computer and that outputs the information indicating the microbiome status of the user.

In a further embodiment, the present invention provides a method for recommending lifestyle changes to a subject. The modification in lifestyle in the subject may be any change, for ex., a change in diet, more exercise/physical activity, different working and/or living environment etc. Modifying a lifestyle of the subject also includes indicating a choice by the subject to change his/her lifestyle, for ex. His or her preference to do more exercise or stopping too much drinking per session. The subject's preferences or choices can thus be accounted for when providing these recommendations to maintain or improve his gut microbiome status.

In one embodiment, the method further comprises combining the level of the one or more biomarkers, such as those obtained by the subject in other health-screenings or health-checkups, with one or more anthropometric measures and/or lifestyle characteristics of the subject or other features already used here in the questionnaire. Whilst such individual health biomarkers may have predictive value in the methods of the present invention, the accuracy of the methods and the recommendations advise may be improved by combining values from multiple biomarkers. By way of an example only, the anthropometric measure is selected from the group in the questionnaire consisting of gender, weight, height, age and body mass index, and the lifestyle characteristic is whether the subject is a alcohol drinker or a non-drinker, whether the subject is a smoker or non-smoker, which is then combined with other health biomarkers such as cholesterol or blood pressure levels towards further increasing the performance of the prediction models.

The methods described herein may be implemented as a computer program running on general purpose hardware, such as one or more computer processors. In some embodiments, the functionality described herein may be implemented by a device such as a smartphone, a tablet terminal or a personal computer. In one aspect, the present invention provides a computer program product comprising computer implementable instructions for causing a programmable computer to predict the microbiome status based on the levels of features obtained from a questionnaire or linked devices as described herein.

In another aspect, the present invention provides a computer program product comprising computer implementable instructions for causing a device to predict the microbiome status given the levels of one or more biomarkers from the user. The computer program product may also be given anthropometric measures and/or lifestyle characteristics from the user. As described herein, anthropometric measures include age, weight, height, gender and body mass index and lifestyle characteristics include smoking status, stress status, anxiety status, depression status, physical activity/exercise frequency etc.

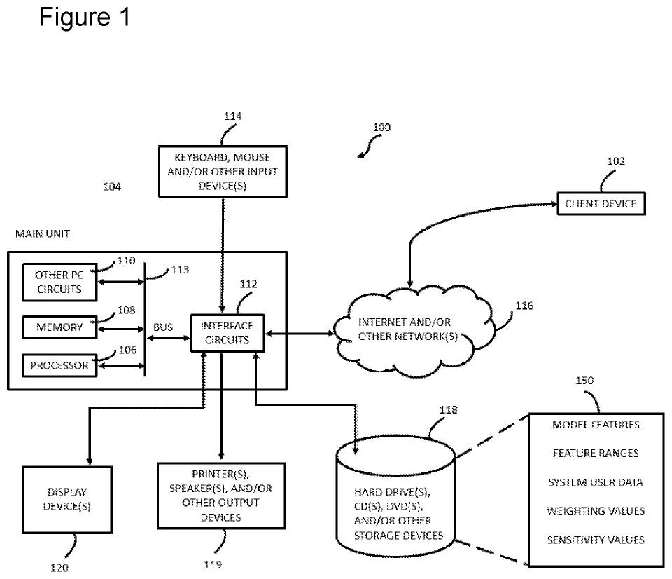

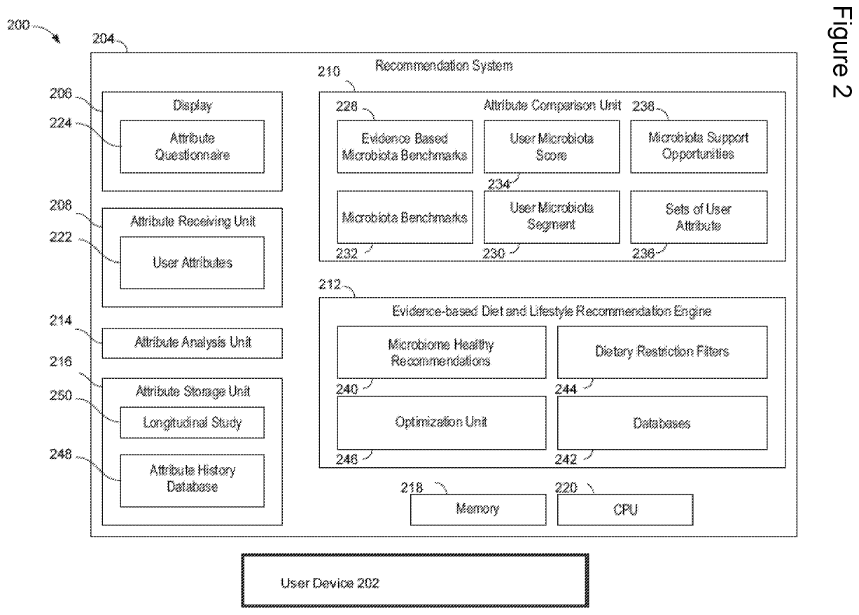

Referring now to FIG. 1, a block diagram is illustrated showing an example of the electrical systems of a host device 100 usable to implement at least portions of the computerized prediction and recommendation system disclosed herein.

In one embodiment, the device 100 illustrated in FIG. 1 corresponds to one or more servers and/or other computing devices that provide some or all of the following functions: (a) enabling access to the disclosed system by remote users of the system; (b) serving web page(s) that enable remote users to interface with the disclosed system; (c) storing and/or calculating underlying data, such as prediction models and recommendation algorithms, the features used by these models and algorithms, and feature values used for decision making by these models and algorithms, needed to implement the disclosed system; (d) calculating and displaying component; and/or (e) making personalized recommendations and advise of various features that can be reduced or enhance to help individuals reach an optimal microbiome status.

In the example architecture illustrated in FIG. 1, the device 100 includes a main unit 104 which preferably includes one or more processors 106 electrically coupled by an address/data bus 113 to one or more memory devices 108, other computer circuitry 110, and/or one or more interface circuits 112. The one or more processors 106 may be any suitable processor, such as a microprocessor from the INTEL PENTIUM® or INTEL CELERON® family of microprocessors. PENTIUM® and CELERON® are trademarks registered to Intel Corporation and refer to commercially available microprocessors. It should be appreciated that in other embodiments, other commercially available or specially designed microprocessors may be used as processor 106. In one embodiment, processor 106 is a system on a chip (“SOC”) designed specifically for use in the disclosed system.

In one embodiment, device 100 further includes memory 108. Memory 108 preferably includes volatile memory and non-volatile memory. Preferably, the memory 108 stores one or more software programs that interact with the hardware of the host device 100 and with the other devices in the system as described below. In addition, or alternatively, the programs stored in memory 108 may interact with one or more client devices such as client device 102 (discussed in detail below) to provide those devices with access to media content stored on the device 100. The programs stored in memory 108 may be executed by the processor 106 in any suitable manner.

The interface circuit(s) 112 may be implemented using any suitable interface standard, such as an Ethernet interface and/or a Universal Serial Bus (USB) interface. One or more input devices 114 may be connected to the interface circuit 112 for entering data and commands into the main unit 104. For example, the input device 114 may be a keyboard, mouse, touch screen, track pad, track ball, isopoint, and/or a voice recognition system. In one embodiment, wherein the device 100 is designed to be operated or interacted with only via remote devices, the device 100 may not include input devices 114. In other embodiments, input devices 114 include one or more storage devices, such as one or more flash drives, hard disk drives, solid state drives, cloud storage, or other storage devices or solutions, which provide data input to the host device 100. In one embodiment, the system is configured to integrate with one or more input devices 114 that are personal mobile devices carried by users. For example, a user wearing a pedometer or activity tracker could provide data from those devices to the system, which could input the exercise amount values accordingly.

One or more storage devices 118 may also be connected to the main unit 104 via the interface circuit 112. For example, a hard drive, CD drive, DVD drive, flash drive, and/or other storage devices may be connected to the main unit 104. The storage devices 118 may store any type of data used by the device 100, including data regarding preferred input features of the models and their decision making ranges, data regarding responses possible for each of the questions for the sets of questionnaires, data regarding users of the system, data regarding previously-generated microbiome assessment statuses, data regarding previously-generated suggestions or recommendations, individual user preferences for input features or sets of features, which they can willingly work to improve upon or not and any other appropriate data needed to implement the disclosed system, as indicated by block 150.

In several embodiments, the Recommendation System indicated by block 150 may store different database models which may include: a Low vs. notLow model, a High vs. notHigh model, a Low vs. High model; a consensus model scoring module (for example, to give a final prediction); and/or optimization module (for example, to provide the most confident results); a recommendations module (for example, to provide users personalized advise on how to maintain or improve their microbiome status), a constraints module (for example, to incorporate the restrictions from the user's side) and a final recommendations module (for example, to incorporate multiple model inputs and user constraints into account).

Alternatively, or in addition, storage devices 118 may be implemented as cloud-based storage, such that access to the storage 118 occurs via an internet or other network connectivity circuit such as an Ethernet circuit 112.

One or more displays 120, and/or printers, speakers, or other output devices 119 may also be connected to the main unit 104 via the interface circuit 112. The display 120 may be a liquid crystal display (LCD), a suitable projector, or any other suitable type of display. The display 120 generates visual representations of various data and functions of the host device 100 during operation of the host device 100. For example, the display 120 may be used to display information about the placement of an individual's microbiome status in the distribution seen for reference population, the deep analyses of the individual's responses to the sets of questionnaires, and associated recommendations to maintain or improve the microbiome status. By way of example only, as shown in FIG. 12 there is individual user's information in response to a set of questionnaires. In FIG. 13 there is the results display on the prediction of the said user's microbiome status. In FIG. 14, based on the user's responses, it shows the features which positively or negatively affect his microbiome status. In FIG. 15, there is a personalized recommendation and advise for maintaining or improving the microbiome-status and instructions on how to follow it.

In the illustrated embodiment, the users of the computerized recommendation system interact with the device 100 using a suitable client device, such as client device 102. The client device 102 in various embodiments is any device that can access content provided or served by the host device 100. For example, the client device 102 may be any device that can run a suitable web browser to access a web-based interface to the host device 100. Alternatively or in addition, one or more applications or portions of applications that provide some of the functionality described herein may operate on the client device 102, in which case the client device 102 is required to interface with the host device 100 merely to access data stored in the host device 100, such as previously described.

In one embodiment, this connection of devices (i.e., the device 100 and the client device 102) is facilitated by a network connection over the Internet and/or other networks, illustrated in FIG. 1 by cloud 116. The network connection may be any suitable network connection, such as an Ethernet connection, a digital subscriber line (DSL), a WiFi connection, a cellular data network connection, a telephone line-based connection, a connection over coaxial cable, or another suitable network connection.

In one embodiment, host device 100 is a device that provides cloud-based services, such as cloud-based authentication and access control, storage, streaming, and feedback provision. In this embodiment, the specific hardware details of host device 100 are not important to the implementer of the disclosed system-instead, in such an embodiment, the implementer of the disclosed system utilizes one or more Application Programmer Interfaces (APIs) to interact with host device 100 in a convenient way, such as to enter information about the user's inputs to the set of questionnaires, to provide his preferences or constraints, and other interactions described in more detail below.

Access to device 100 and/or client device 102 may be controlled by appropriate security software or security measures. An individual user's access can be defined by the device 100 and limited to certain data and/or actions, such as selecting various options to the questions or viewing predicted values, according to the individual's responses. Other users of either host device 100 or client device 102 may be allowed to alter other data, such as weighting, sensitivity, or feature range values, depending on those users' identities. Accordingly, users of the system may be required to register with the device 100 before accessing the content provided by the disclosed system.

In a preferred embodiment, each client device 102 has a similar structural or architectural makeup to that described above with respect to the device 100. That is, each client device 102 in one embodiment includes a display device, at least one input device, at least one memory device, at least one storage device, at least one processor, and at least one network interface device. It should be appreciated that by including such components, which are common to well-known desktop, laptop, or mobile computer systems (including smart phones, tablet computers, and the like), client device 102 facilitates interaction among and between each other by users of the respective systems.

In various embodiments, devices 100 and/or 102 as illustrated in FIG. 1 may in fact be implemented as a plurality of different devices. For example, the device 100 may in actuality be implemented as a plurality of server devices operating together to implement the media content access system described herein. In various embodiments, one or more additional devices, not shown in FIG. 1, interact with the device 100 to enable or facilitate access to the system disclosed herein. For example, in one embodiment the host device 100 communicates via network 116 with one or more public, private, or proprietary repositories of information, such as public, private, or proprietary repositories of microbiome-friendly or other advises, recommendations, nutritional information, nutrient content information, menu planners, recipe databases, healthy range information, energy information, environmental impact information, or the like.

In one embodiment, the disclosed system does not include a client device 102. In this embodiment, the functionality described herein is provided on host device 100, and the user of the system interacts directly with host device 100 using input devices 114, display device 120, and output devices 119. In this embodiment, the host device 100 provides some or all of the functionality described herein as being user-facing functionality.

In various embodiments, the system disclosed herein is arranged as a plurality of modules, wherein each module performs a particular function or set of functions. The modules in these embodiments could be software modules executed by a general purpose processor, software modules executed by a special purpose processor, firmware modules executing on an appropriate, special-purpose hardware device, or hardware modules (such as application specific integrated circuits (“ASICs”)) that perform the functions recited herein entirely with circuitry. In embodiments where specialized hardware is used to perform some or all of the functionality described herein, the disclosed system may use one or more registers or other data input pins to control settings or adjust the functionality of such specialized hardware.

A user's goal to predict his microbiome status may be examined over time to detect potential problematic patterns or improvements. The system can be used to then identify the recommended shifts needed in the habits, food items, supplements, menus, or recipes in order to get closer to better microbiome status. In some embodiments, the system and methods disclosed herein can be used by nutritionists, health-care professionals, and individual users.

FIG. 2 illustrates a microbiome recommendation system according to an embodiment of the present disclosure. The system 200 includes a user device 202 and a recommendation system 204. In another embodiment of the present disclosure, the recommendation system 204 can be one example of the embodiment of the recommendation system 150 of FIG. 1. The user device 202 may be implemented as a computing device, such as a computer, smartphone, tablet, smartwatch, or other wearable through which an associated user can communicate with the recommendation system 204. The user device 202 may also be implemented as, e.g., a voice assistant configured to receive voice requests from a user and to process the requests either locally on a computer device proximate to the user or on a remote computing device (e.g., at a remote computing server).

The recommendation system 204 includes one or more of a display 206, an attribute receiving unit 208, an attribute comparison unit 210, an evidence-based assessment and recommendation engine 212, an attribute analysis unit 214, an attribute storing unit 216, a memory 218, and a CPU 220. Note, that in some embodiments, a display 206 may additionally or alternatively be located within the user device 202. In an example, the recommendation system 204 may be configured to receive a request for a plurality of microbiome-healthy recommendations 240. For example, a user may install an application on the user device 202 that requires the user to sign up for a recommendation service. By signing up for the service, the user device 202 may send a request for the microbiome-healthy recommendations 240. In a different example, the user may use the user device 202 to access a web portal using user-specific credentials. Through this web portal, the user may cause the user device 202 to request microbiome healthy recommendations from the recommendation system 204.

In another example, the recommendation system 204 may be configured to request and receive a plurality of user attributes 222. For example, the display 206 may be configured to present an attribute questionnaire 224 to the user. The attribute receiving unit 208 may be configured to receive the user attributes 222. In one example, the attribute receiving unit 208 may receive a plurality of answers 226 based on the attribute questionnaire 224, and based on the plurality of answers, determine the plurality of user attributes 222. For example, the attribute receiving unit 208 may receive answers to the attribute questionnaire 224 suggesting that the habits of the user are good or not good for the microbiome and then suggest the user attributes 222 to be maintain or improved upon for microbiome. In another example, the user device attribute receiving unit 208 may directly receive the user attributes 222 from the user device 102.

In another example, the attribute receiving unit 208 may be configured to receive the test results of a home-test kit, the results of a standardized health test administered by a medical professional, the results of this self-assessment tool used by the user, or the results of any external or third party test. Based on the results from any of these tests or tools, the attribute receiving unit 208 may be configured to determine the user attributes 222. For example, the microbiome health status of the user may be determined before the intervention of the microbiome-healthy recommendations by predicting the alpha-diversity of microbiota species. The same measurements may be predicted again at a time period after the microbiome-healthy interventions to determine whether there has been an improvement or maintenance of the microbiome health status of the user.

The recommendation system 204 may be further configured to compare the plurality of user attributes 222 to a corresponding plurality of evidence-based microbiome-healthy benchmarks 228.

Furthermore, the attribute comparison unit 210 may be further configured to determine a microbiota benchmark set 232 based on the user's microbiota segment 230. For example, if the attribute comparison unit 210 determines that a user falls into the obese BMI segment 230, based on the plurality of user attributes 222, the attribute comparison unit 210 may select a microbiota benchmark set 232 that has been created and defined according to the specific needs for a healthy microbiome.

The comparison unit 210 may be further configured to select, from this determined microbiome benchmark set 232, the evidence-based microbiota benchmarks 128 and compare the now selected evidence-based microbiota benchmarks 228 to each of the corresponding user attributes 222. For example, when the microbiota benchmark set 232 has been determined, in response to the determination, the attribute comparison unit 210 may compare a user attribute 222 that represents the user's antibiotics intake to an evidence-based microbiota benchmark 228 that represents a benchmark antibiotics intake, determining whether the user is below, at, or above the benchmark antibiotics intake, from the microbiome perspective. Though this example is based on a concrete, numerical comparison, another example of a benchmark comparison may be qualitative and different depending on a person. For example, a user attribute 222 may indicate that the user is currently engaging in lower than normal levels of exercise. An example benchmark related to a user exercise level may indicate that an average or higher level of exercise is desired and thus, the user attribute 222 indicating a lower level of exercise is determined to be below that of the benchmark. As different users engage in differing levels of exercise, even under the same circumstances, such a comparison requires a customized approach.

In addition, during the comparison from the prior example, the attribute comparison unit 210 may be configured to determine a user microbiota score 234 based on the comparison between the evidence-based microbiota benchmarks 228 and the user attributes 222. For example, the attribute comparison unit 210 may determine a user microbiota score of 95/100 if the user attributes 222 very nearly meet all or most of the corresponding evidence-based microbiota benchmarks 228. In another example, a score may be represented through lettering grades, symbols, or any other system of ranking, for example, “Low”, “Medium”, “High” that allows a user to interpret how well their current attributes rate amongst benchmarks. This user microbiota score 234 may be presented through the display 206.

The recommendation system 204 may be further configured to determine a plurality of microbiota support opportunities 238 based on the plurality of user attributes 222 and the comparison to the corresponding plurality of evidence-based microbiota benchmarks 228. In one example, the attribute comparison unit 210 may determine microbiota support opportunities 238 for every user attribute 222 that does not meet the corresponding evidence-based microbiota benchmark. In this example, a corresponding evidence-based microbiota benchmark 228 may require a user have an intake of 2 ug/day of folate, whereas the user attribute may indicate the user is only receiving 1 ug/day of folate. Therefore, the attribute comparison unit 210 may determine an increase in folate intake to be a microbiota support opportunity 238.

In another example, the attribute comparison unit 210 may be configured to identify a first set of user attributes 236 comprised of each of the plurality of user attributes 222 that are below the corresponding one of the plurality of evidence-based microbiota benchmarks 228 as well as identify a second set of user attributes 236 comprised of each of the plurality of user attributes 222 that are greater than or equal to the corresponding evidence-based microbiota benchmarks 228. While the first set of user attributes 236 is determined similarly to the above given example, the second set of user attributes 236 differs in that, although the associated user does not appear to have a deficiency, there may be opportunities to support microbiome health by recommending the user maintain current practices or opportunities to further improve upon them. Accordingly, the recommendation system 204 may determine opportunities to support microbiome health based on which attributes 222 populate either sets 236.

The recommendation system 204 may be further configured to identify a plurality of microbiome-healthy recommendations 240 based on the plurality of microbiota support opportunities 238. For example, the evidence-based diet and lifestyle recommendation engine 212 may be configured to be cloud-based. The recommendation engine 212 may comprise one or more of a plurality of databases 242, a plurality of dietary restriction filters 244, and an optimization unit 246. Based on the plurality of opportunities 238, the recommendation engine 212 may identify the plurality of microbiome-healthy recommendations 240 according to the one or more of plurality of databases 242, the dietary restriction filters 244, and the optimization unit 246.

In another example, the recommendation system 204 may be configured to provide continuous recommendations, based on prior user attributes. For example, the recommendation system 204 may comprise, in addition to the previously discussed elements, an attribute storing unit 216 and an attribute analysis unit 214. The attribute storing unit 216 may be configured to, responsive to the attribute receiving unit 108 receiving the plurality of user attributes 222, add the received user attributes 222 to an attribute history database 248 as a new entry based on when the plurality of user attributes 222 were received. For example, if user attributes 222 are received by the attribute receiving unit 208 on a first day, the attribute storing unit 216 will add the received user attributes 222 to a cumulative attribute history database 248 noting the date of entry, in this case the first day. Later, if user attributes 222 are received by the attribute receiving unit 208 on a second day, e.g. the next day, the attribute storing unit 216 will also add these new attributes to the attribute history database 248, noting that they were received on the second day, while also preserving the earlier attributes from the first day.

This attribute analysis unit 214 may be configured to analyze the plurality of user attributes 222 stored within the attribute history database 248, wherein analyzing the stored plurality of user attributes 222 comprises performing a longitudinal study 250. Continuing the earlier example, the attribute analysis unit 214 may perform a longitudinal study of the user attributes 222 from each of the first day, the second day, and every other collection of user attribute 222 found within the attribute history database 248. The evidence based diet and lifestyle recommendation engine 212 may be further configured to generate a plurality of microbiome-healthy recommendations 240 based on at least the stored user attributes 222 found within the attribute history database 248 and the analysis performed by the attribute analysis unit 214.

In an embodiment, the attribute analysis unit 214 is further configured to repeatedly analyze the plurality of user attributes 222 stored within the attribute history database 248 responsive to the attribute storing unit 216 adding a new entry to the attribute history database 248, essentially re-analyzing all of the data within the attribute history database 248 immediately after new user attributes 222 are received. Similarly, the evidence based diet and lifestyle recommendation engine 212 may be further configured to repeatedly generate the plurality of microbiome-healthy recommendations 240 responsive to the attribute analysis unit 214 completing an analysis, thereby effectively generating new microbiome-health recommendations 240 that consider all past and present user attributes 222 each time a new set of user attributes 222 is received.

In various embodiments, the user-specific (or population-specific) inputs to the disclosed system are programmable and configurable, and include gender, age, weight, height, physical activity level, whether obese, and the like.

In further embodiments, individuals may provide their own weighting values tailored to their own personal choices and health conditions. With these personalized ranges and/or weighting values, the disclosed system can then calculate a completely personalized advice for maintaining or improving the individual's microbiome status.

In an embodiment, the disclosed system includes or is connected to a database containing food items, menus or recipes and respective nutrient content. In this embodiment, the disclosed system includes a fuzzy search feature that enables a user to enter a consumed (or to-be consumed) food, and thereafter searches the database to find a closest item to the user-provided item. The disclosed system, in this embodiment, uses stored nutritional information about the matched food item to determine whether it is a microbiome-friendly item.

In various embodiments, the disclosed system further includes an interface (e.g., a graphical user interface) to display the amount of each nutrient available in each food composing the diet and displays the amount of energy available to be consumed. In some embodiments, this interface enables users to modify the amount of various foods or energy to be consumed. In other embodiments, the system is configured to determine amounts of food or energy consumed using non-user-input data, such as by scanning one or more bar codes, QR codes, or RFID tags, image recognition systems, or by tracking items ordered from a menu or purchased at a grocery store.

Various embodiments of the disclosed system display a dashboard or other appropriate user interface to a user that is customized based on the user's needs. In embodiments of the system disclosed herein, a graphical user interface is provided which advantageously enables, for the first time, users to input data about his responses to the sets of questionnaires and for him to see an indication of a score, based appropriately on prediction, that reflects overall placement of his status in the generally seen distribution of microbiome.

All the disclosed methods and procedures described in this disclosure can be implemented using one or more computer programs or components. These components may be provided as a series of computer instructions on any conventional computer readable medium or machine-readable medium, including volatile and non-volatile memory, such as RAM, ROM, flash memory, magnetic or optical disks, optical memory, or other storage media. The instructions may be provided as software or firmware and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

As noted above, the disclosed system in some embodiments relies on one or more modules (hardware, software, firmware, or a combination thereof) to perform various functionalities discussed above.

The inventors have shown that a predictive tool can be created that is based on features obtained from questionnaire, such as for health, lifestyle, dietary habits or preferences, and that allows to predict the gut microbiome status, for example, microbiome diversity. Furthermore, where known equivalents exist to specific features, such equivalents are incorporated as if specifically referred in this specification. Further advantages and aspects of the present invention are apparent from the figures and non-limiting examples.

In several embodiments, the invention provides a method for determining the gut microbiome status comprising:
(i) determining the gut microbiome status in a subject and
(ii) providing recommendations to improve or maintain microbiome status in said subject.

In several embodiments, the invention provides that the determination of gut microbiome status is by a questionnaire to predict the microbiome diversity of the subject.

In one embodiment, the invention provides that the determination of gut microbiome status is additionally by a biological sample to quantify the microbiome diversity of said subject.

In several embodiments, the methods of the invention are computer-implemented.

In several embodiments, the methods of the invention evaluate feature parameters related to gut microbiome status of a subject.

In several embodiments, the feature parameters related to gut microbiome status are selected from the group comprising:
(i) geographic country of residence of the subject comprising specific latitude and longitude;
(ii) antibiotic use comprising whether and when antibiotics have been used in the past 1 year;
(iii) medication usage comprising whether and when medications have been used in the last 12 months
(iv) anthropometric data comprising age, weight, height, body mass index, gender;
(v) alcohol consumption comprising the type of alcohol, amount and frequency of consumption;
(vi) smoking status
(vii) exercise and/or physical activity comprising the location of exercise as indoor or outdoor, frequency, and duration;
(viii) ethnicity;
(ix) season;
(x) travel comprising the location of travel and duration;
(xi) sleep comprising duration in hours and/or sleep quality;
(xii) stress status, anxiety status and/or depression status;
(xiii) history of diseases comprising chicken pox, irritable bowel disease, or diabetes
(xiv) history of allergies comprising seasonal or food allergies and/or food intolerances;
(xv) vaccination status comprising flu vaccine or pneumococcal vaccine.
(xvi) nutritional supplement use comprising vitamin or mineral supplements;
(xvii) source of drinking water;
(xviii) personal hygiene comprising flossing of teeth, use of deodorant, use of cosmetics; and
(xix) type of food consumption comprising intake of vegetable, fruit, fermented food and/or whole grains, amount and frequency of consumption.

In several embodiments, the feature parameters related to gut microbiome status are selected from the group consisting of:
(i) geographic country of residence of the subject comprising specific latitude and longitude;
(ii) antibiotic use comprising whether and when antibiotics have been used in the past 1 year;
(iii) medication usage comprising whether and when medications have been used in the last 12 months
(iv) anthropometric data comprising age, weight, height, body mass index, gender;
(v) alcohol consumption comprising the type of alcohol, amount and frequency of consumption;
(vi) smoking status
(vii) exercise and/or physical activity comprising the location of exercise as indoor or outdoor, frequency, and duration;
(viii) ethnicity;
(ix) season;
(x) travel comprising the location of travel and duration;
(xi) sleep comprising duration in hours and/or sleep quality;
(xii) stress status, anxiety status and/or depression status;
(xiii) history of diseases comprising chicken pox, irritable bowel disease, or diabetes
(xiv) history of allergies comprising seasonal or food allergies and/or food intolerances;
(xv) vaccination status comprising flu vaccine or pneumococcal vaccine.
(xvi) nutritional supplement use comprising vitamin or mineral supplements;
(xvii) source of drinking water;
(xviii) personal hygiene comprising flossing of teeth, use of deodorant, use of cosmetics; and
(xix) type of food consumption comprising intake of vegetable, fruit, fermented food and/or whole grains, amount and frequency of consumption.

In several embodiments of the invention, the method involves the steps of:
(i) determining at least one feature parameter related to gut microbiome status in a subject;
(ii) comparing at least one feature parameter related to gut microbiome status in said subject to a population database of subjects from the same geographical region; and
(iii) determining whether a subject is low, medium or high on at least one feature parameter.

In several embodiments, the subject is informed of their gut microbiome status on a computer interface such as shown in FIGS. 1 and 2.

In several embodiments, the systems and methods of the invention contribute to maintaining and improving the microbiome status by providing microbiome healthy recommendations such as nutritional supplements, diet recommendations, menu recommendations and recipe recommendations to improve or maintain the alpha diversity of microbial species in the intestine.

In one embodiment of the invention, microbiome health improvements or maintenance of microbiome health can be determined from a biological sample taken from the subject, before and after the dietary recommendations of the present invention, by measurement of parameters diversity of microbial species in the intestine. Thus, it can be determined over time, the microbiome-healthy improvements after the individual has followed the diet, menu and recipe recommendations of the present invention.

In various embodiments, the system disclosed herein provides recommendations of supplements, food items, menus or recipes indicating the nutritional impact for the microbiome. In these embodiments, the system determines and stores one or more indications of the needs of the individual for whom the recommendations are being calculated, for an individual over a given period of time such as a meal, an entire day, a week or a month.

In one embodiment of the invention, the methods and systems of the invention comprise recommendations for food or nutrient groups selected from the group consisting of:
(i) wholegrain foods;
(ii) beans and legumes;
(iii) fiber;
(iv) nuts and seeds; and
(v) omega-3 fatty acids.

In one embodiment of the invention, the methods and systems of the invention comprise recommendations for food or nutrient group comprises recommendations of meal plans or recipes containing:
(i) wholegrain foods;
(ii) beans and legumes;
(iii) fiber;
(iv) nuts and seeds; and
(v) omega-3 fatty acids.

Those skilled in the art will understand that they can freely combine all aspects of the present invention disclosed herein, without departing from the scope of the invention as disclosed. Further, aspects described for different embodiments of the present invention may be combined. Although the invention has been described by way of example, it should be appreciated that variations and modifications may be made without departing from the scope of the invention as defined in the claims.

As used in this specification, the words “comprises”, “comprising”, and similar words, are not to be interpreted in an exclusive or exhaustive sense. In other words, they are intended to mean “including, but not limited to”.

The above description is exemplary of the aspects of the system disclosed herein. As noted, the disclosed systems and methods could be used to predict the microbiome status of an individual as defined by other microbiome indices not mentioned here and also indicate the impact of different factors such as other intrinsic, extrinsic or environmental factors not mentioned here based on any appropriate measurable characteristic, and the disclosed systems and methods are not limited to determining only the microbiome status as defined here and not limited to delineating the impact of factors on the microbiome as listed here. Moreover, the functionality of the above-described system is not limited to the functionalities indicated herein. It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

Various preferred features and embodiments of the present invention are described below by way of non-limiting examples.

### EXAMPLES

### Example 1: Building Models to Predict Microbiome Status

AGP data is a publicly available dataset containing microbiota data for 9511 subjects, with associated metadata features related to general survey questions such as on individual traits, lifestyle, food habits and medical conditions (about 200 questions in total). Further details of this study and links to this dataset are available in the AGP publication (McDonald D, et al. mSystems. 2018).

Predictive models were built to determine the microbiome status of an individual subject. In particular, the model predicted the alpha diversity of the microbiome by a number of feature parameters to determine whether a subject belongs “Low” or “notLow”; “High” or “notHigh”; “Low” or “High”; categories as defined above.

For building the classification models, the data was split into a training set “Train” and a testing set “holdout/Test set”. For optimal model performance we used down sampling to balance the imbalanced classes, which may occur based on the definition of bins.

The Train set was used by a machine learning algorithm to train the model. This involved finding variables (i.e., features) and thresholds (or coefficients) to use for classifying the groups. The learning from the data was done in a cross-validated manner where Train data was split into partitions with some parts used for training the model and other for internal testing (k-Fold Cross-Validation, for ex., 3-folds), or with this process also repeated a few times (Repeated k-Fold Cross-Validation, for ex., 10-folds, 10-repeats).

The holdout/Test set was used only for checking the performance of the final trained model. This holdout/Test dataset was thus not used during the model training phase. We evaluated multiple statistical models using freely available tools (R software, python) and identified the best models for Low vs. notLow, High vs. notHigh, and Low vs. High groups or categories.

Evaluating the model performance was critical, during all phases of modeling. Once the model was trained, it was applied on holdout/Test data, which was not used during the training phase. The model computed probabilities to be in each group (e.g. “Low”, “notLow”). A final decision was made based on this probability, which thus required the use of a threshold. This threshold impacted the final classification for a subject, whether a subject was correctly classified or not. Thus, the error was evaluated for different choices of threshold. For each given threshold, a confusion matrix was computed. This confusion matrix essentially listed the number of correctly and incorrectly classified subjects. By using different thresholds, one generated many confusion matrices, which were used to derived sensitivity and specificity at different thresholds. These two metrics—sensitivity and specificity—were commonly shown in the form of a Receiving Operating Curve (ROC); which summarized the model performance over several threshold values.

Receiver Operating Characteristic (ROC) curves were produced for the model. We either defined the group of “Low” subjects (and in “notLow” group) and predicted the probability of subject to be in this group; or, we defined the subjects to be in the “High” group (and in “notHigh” group) and predicted the probability of subject to be in this group; or, we defined the subjects to be in the “Low” group (and in “High” group) and predicted the probability of subject to be in this group.

The data set used for the examples of a predictive model was from the American Gut Project (AG P) database (http://americangut.org).

### Example 2: Low Versus High Gut Microbiome Diversity Model (I)

For the Low vs. High gut microbiota diversity model, the runs were done with these parameters—Train-holdout/Test split of 80-20%, age_years >=20 & age_years <=70, bmi >=18.5 & bmi <=35, country==′USA′—these are the results for the holdout/Test set: accuracy—0.687, 95% confidence interval—(0.6375, 0.7335), balanced accuracy—0.6858, kappa—0.37, sensitivity/prediction of Low class—0.6746, specificity/prediction of High class—0.6971, positive predictive value—0.6441, and negative predictive value—0.7250. The top features used by this model to make a decision about Low or High gut microbiota diversity status were ethnicity, antibiotics usage, age, healthy status (no diabetes, IBD), and BMI.

### Example 3: Low Versus High Gut Microbiome Diversity Model (II)

This model ran with the following parameters: Country: USA, Age: 20-70 years, BMI: 18.5-30. The following results were obtained for Random Forests (RFs)—accuracy—0.6928, 95% confidence interval—(0.6132, 0.7648), balanced accuracy—0.6929, kappa—0.3858, sensitivity/prediction of Low class—0.7105, specificity/prediction of High class—0.6753, positive predictive value—and negative predictive value—0.7027; for Support Vector Machines (SVMs)—accuracy—0.6667, 95% confidence interval—(0.586, 0.7407), balanced accuracy—0.6668, kappa—sensitivity/prediction of Low class—0.6842, specificity/prediction of High class—0.6494, positive predictive value—0.6582, and negative predictive value—0.6757; for DecisionTrees (DTs)—accuracy—0.6078, 95% confidence interval—(0.5237, 0.6857), balanced accuracy—kappa—0.2162, sensitivity/prediction of Low class—0.6579, specificity/prediction of High class—0.5584, positive predictive value—0.5952, and negative predictive value—0.6232. Top features across algorithms (and diversity measures) were: Age, Antibiotic usage, Alcohol consumption, Travel, Exercise.

### Example 4: Low Versus notLow Gut Microbiota Diversity Model (I)

This model ran with these parameters—Train-holdout/Test split of 80-20%, age_years=all ages, bmi=all BM's, country=all countries—these are the results for the holdout/Test set (n=1230): accuracy—0.679, 95% confidence interval—(0.6511, 0.7041), balanced accuracy—0.62530, kappa—0.1845, sensitivity/prediction of Low class—0.54378, specificity/prediction of notLow class—0.70681, positive predictive value—0.28434, and negative predictive value —0.87853

### Example 5: High Versus notHigh Gut Microbiota Diversity Model (I)

This model ran with these parameters—Train-holdout/Test split of 80-20%, age_years=all, bmi =all BM's, country=all countries—these are the results for the holdout/Test set: accuracy—0.5984, 95% confidence interval—(0.5709, 0.6255), balanced accuracy—0.6204, kappa—0.1705, sensitivity/prediction of High class—0.6595, specificity/prediction of notHigh class—0.5812 positive predictive value—0.3072, and negative predictive value—0.8584

### Example 6: Low Versus High Gut Microbiota Diversity Model (III)

This model ran with these parameters—Train-holdout/Test split of 80-20%, age_years=all, bmi=all, country=all—these are the results for the holdout/Test set: accuracy—0.7117, 95% confidence interval—(0.6696, 0.7512), balanced accuracy—0.7038, kappa—0.4103, sensitivity/prediction of Low class—0.6406, specificity/prediction of High class—0.7670, positive predictive value—0.6814, and negative predictive value—0.7329

### Example 7: Low Versus notLow Gut Microbiota Diversity Model (II)

To define “Low”—first/lower quartile on all these three diversity measures, the following cut-offs were used on the American Gut Project (AGP) data: MEAN_OBSERVEDOTU <=88.30 & MEAN_FAITHPD <=10.91 & MEAN_SHANNON <=4.38 To define “notLow” on all these three diversity measures, the cut-offs used on the AGP data were: MEAN_OBSERVEDOTU >88.30 & MEAN_FAITHPD>10.91 & MEAN_SHANNON>4.38

This model ran with these parameters—Bin definition as first/lowest quartile versus rest defined on all three diversity measures, input AGP data with survey minimum response rate of 0.65, with no cut-offs applied on any of the features, using Random Forests algorithm in cross-validation Train mode with 3-folds, post-processing Train size of 2370 and holdout/Test size of 1490. These are the results for the Train set in cross-validation mode: sensitivity—0.65±0.02, specificity—0.63±0.01, accuracy—0.63±0.01, AUC-ROC—0.7±0.02 These are the results for the holdout/Test set: sensitivity—0.67, specificity—0.62, accuracy—0.63, AUC-ROC—0.72

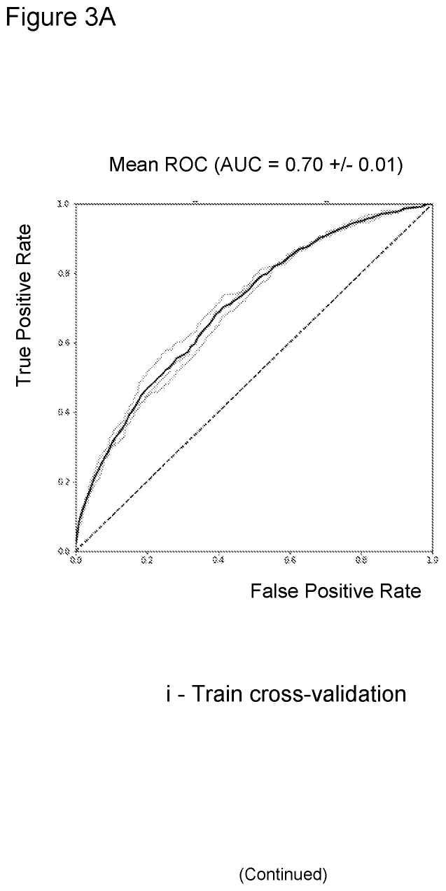

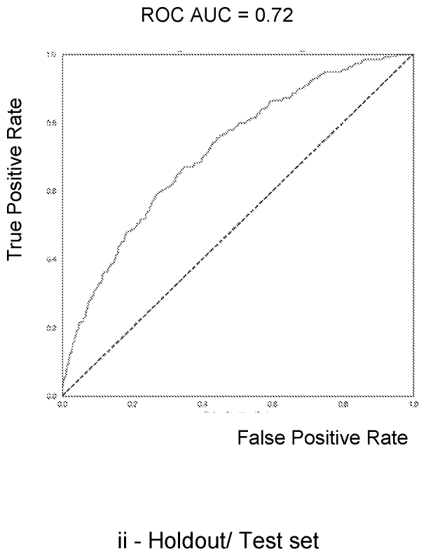

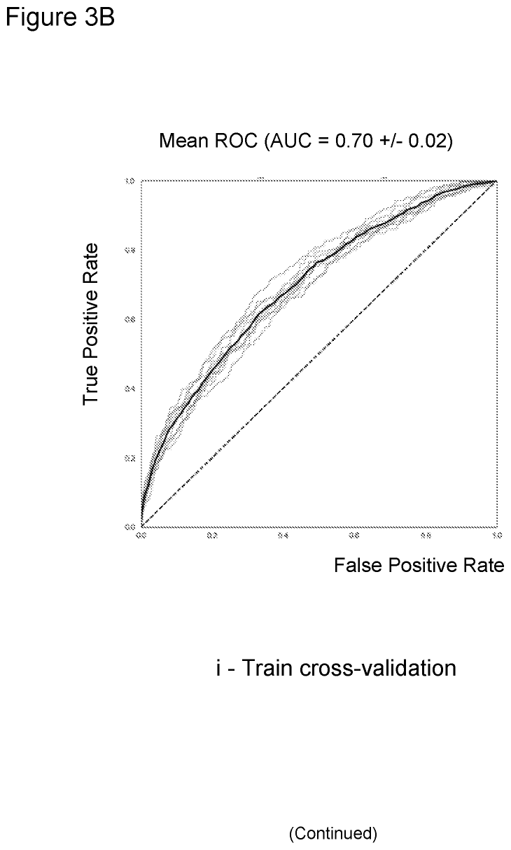

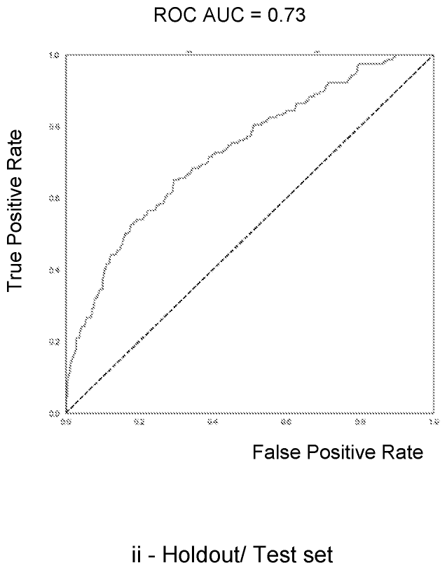

The ROC curves and the AUC values are shown in FIG. 3A.

### Example 8: Low Versus notLow Gut Microbiota Diversity Model (Ill)

This model ran with these parameters—Bin definition as less than (mean −1*std) versus rest defined on all three diversity measures, input AGP data with survey minimum response rate of 0.85, with no cut-offs applied on any of the features, using Random Forests algorithm in cross-validation Train mode with 3-folds, 3-repeats, post-processing Train size of 1234, holdout/Test size of 1560. These are the results for the Train set in cross-validation mode: sensitivity—0.62±specificity—0.65±0.02, accuracy—0.65±0.02, AUC-ROC—0.7±0.02 These are the results for the holdout/Test set: sensitivity—0.62, specificity—0.71, accuracy—0.71, AUC-ROC—0.73

The ROC curves and the AUC values are shown in FIG. 3B both for (i) Train (cross-validation) and (ii) holdout/Test set. FIG. 9 shows the improvement in the performance of the model (AUC values of the ROC curves for Train (cross-validation) and holdout/Test set) as the features deemed important for this model by SHAP analysis were added one by one to be a part of the model.

### Example 9: High Versus notHigh Gut Microbiota Diversity Model (II)

The High, notHigh bins are defined together on three diversity measures: observed OTU, Faith PD, and Shannon. To define “High”—third/upper quartile on all these three diversity measures—the following cut-offs are used on the American Gut Project (AGP) data: MEAN_OBSERVEDOTU >137.8 & MEAN_FAITHPD>15.65 & MEAN_SHANNON>5.5 To define “notHigh” on all the above three diversity measures the cut-offs used on the AGP data are: MEAN_OBSERVEDOTU <=137.8 & MEANFAITHPD <=15.65 & MEAN_SHANNON <=5.5

This model ran with these parameters—Bin definition as third/upper quartile vs rest defined on all three diversity measure, input AGP data with survey min response rate of 0.65, with no cut-offs applied on any of the features, Random Forests algorithm in cross-validation Train mode with 3-folds, post processing Train size: 2564, holdout/Test size: 1520 These are the results for the Train set in cross-validation mode: sensitivity—0.6±0.04, specificity—0.68±0.02, accuracy—0.67±AUC-ROC—0.71±0.01 These are the results for the holdout/Test set: sensitivity—0.66, specificity—0.66, accuracy—0.66, AUC-ROC—0.73

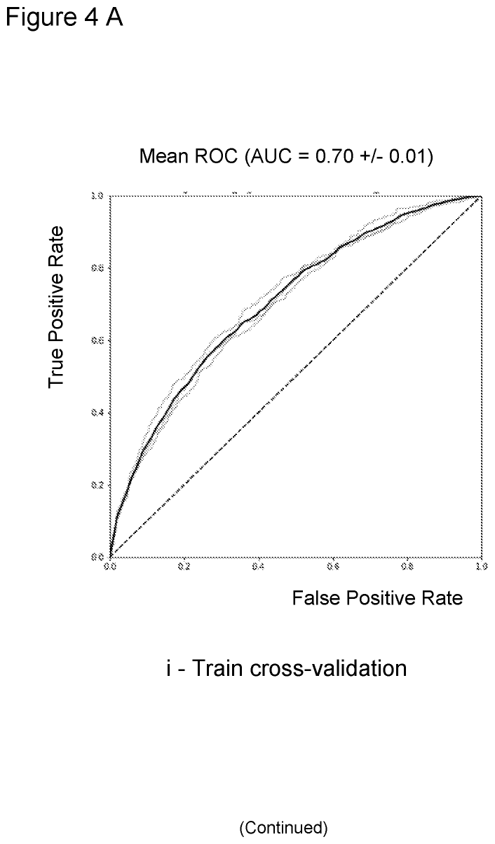

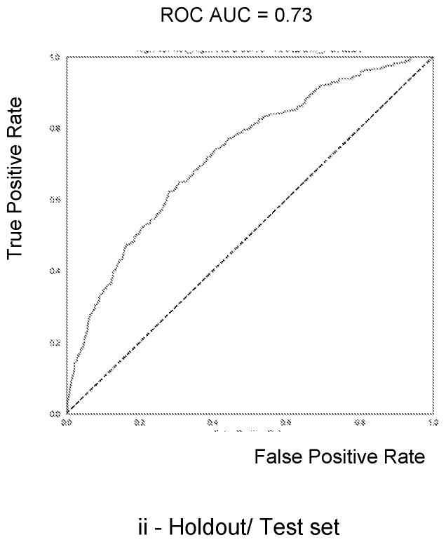

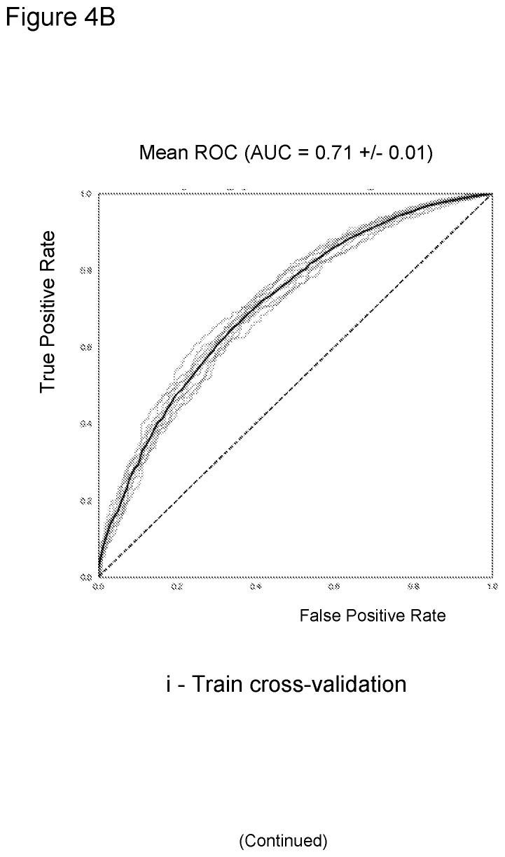

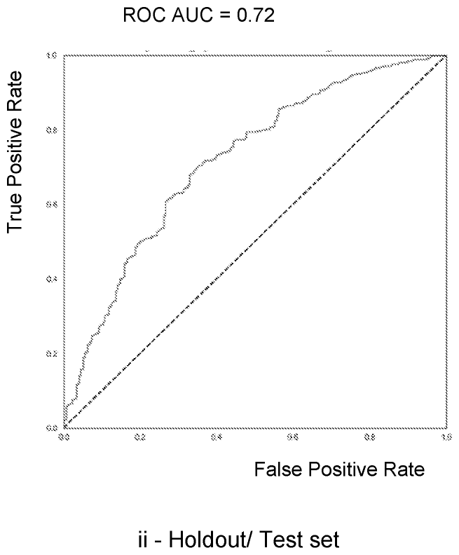

The ROC curves and the AUC are shown in FIG. 4A.

### Example 10: High Versus notHigh Gut Microbiota Diversity Model (Ill)

This model ran with these parameters—Bin definition as more than (mean+1*std) versus rest defined on all three diversity measures, input AGP data with survey minimum response rate of with no cut-offs applied on any of the features, using Random Forests algorithm in cross-validation Train mode with 3-folds, 3-repeats, post-processing Train size of 1408, holdout/Test size of 1585. These are the results for the Train set in cross-validation mode: sensitivity—0.69±specificity—0.61±0.03, accuracy—0.68±0.01, AUC-ROC 0.71±0.02—These are the results for the holdout/Test set: sensitivity—0.71, specificity—0.64, accuracy—0.70, AUC-ROC—

The ROC curves and the AUC values are shown in FIG. 4B both for (i) Train (cross-validation) and (ii) holdout/Test set. FIG. 10 shows the improvement in the performance of the model (AUC values of the ROC curves for Train (cross-validation) and holdout/Test set) as the features deemed important for this model by SHAP analysis were added one by one to be a part of the model.

### Example 11: Low Versus High Gut Microbiota Diversity Model (IV)

The Low and High bins are defined together on three diversity measures: observed OTU, Faith PD, and Shannon. To define “Low”—first/lower quartile on all these three diversity measures—the following cut-offs are used on the American Gut Project (AGP) data are: MEAN_OBSERVEDOTU <=88.30 & MEAN_FAITHPD <=10.91 & MEAN_SHANNON <=4.38 To define “High”—third/upper quartile on all the above three diversity measures—the cut-offs used on the AGP data are: MEAN_OBSERVEDOTU >137.8 & MEAN_FAITHPD>15.65 & MEAN_SHANNON>5.5

The model ran with these parameters—Bin definition as first/lowest quartile vs third/upper quartile defined on all three diversity measures, input AGP data with survey minimum response rate of 0.65, with no cut-offs applied on any of the features, using Random Forests algorithm in cross-validation Train mode of 3-folds, post-processing Train size of 2370, holdout/Test size of 617. These are the results for the Train set in cross-validation mode: sensitivity—0.73±0.01, specificity—0.67±0.03, accuracy—0.7±0.02, AUC-ROC—0.78±0.02 These are the results for the holdout/Test set: sensitivity—0.71, specificity—0.71, accuracy—0.71, AUC-ROC—0.79

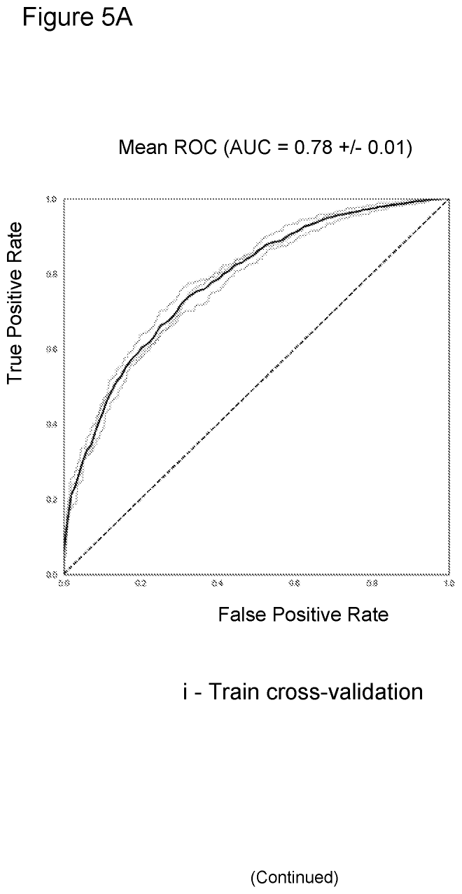

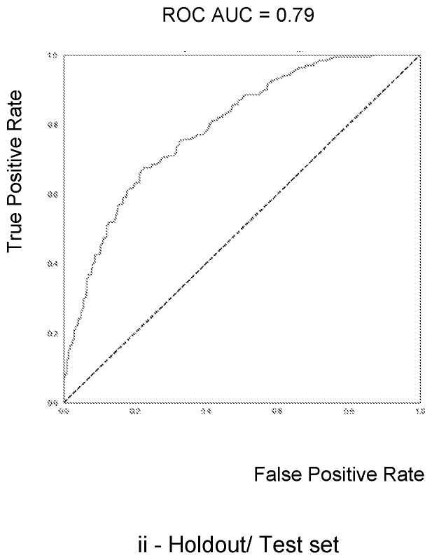

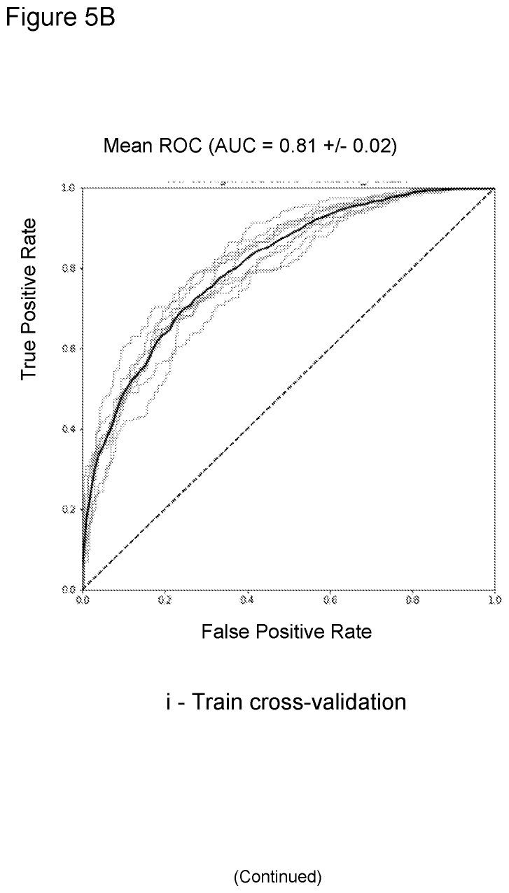

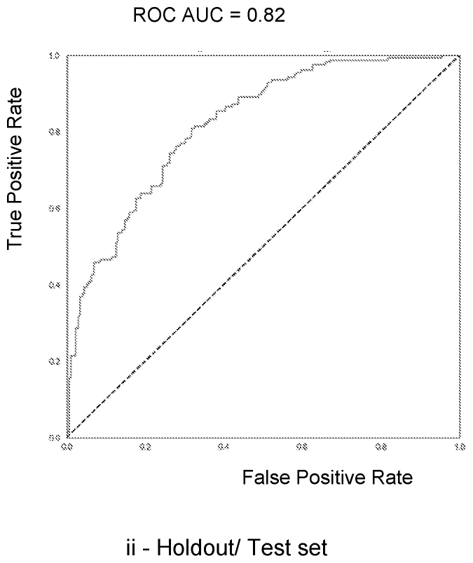

The ROC curves and the AUC values are shown in FIG. 5A.

### Example 12: Low Versus High Gut Microbiota Diversity Model (V)

This model ran with these parameters—Bin definition as less than (mean−1*std) versus more than (mean+1*std) defined on all three diversity measures, input AGP data with survey minimum response rate of 0.85, with no cut-offs applied on any of the features, using Random Forests algorithm in cross-validation Train mode with 3-folds, 3-repeats, post-processing Train size of 1232, holdout/Test size of 331. These are the results for the Train set in cross-validation mode: sensitivity—0.73±0.03, specificity—0.72±0.03, accuracy—0.73±0.02, AUC-ROC—0.81±0.03 These are the results for the holdout/Test set: sensitivity—0.72, specificity—0.74, accuracy—0.73, AUC-ROC—0.82

The ROC curves and the AUC values are shown in FIG. 5B both for (i) Train (cross-validation) and (ii) holdout/Test set. FIG. 11 shows the improvement in the performance of the model (AUC values of the ROC curves for Train (cross-validation) and holdout/Test set) as the features deemed important for this model by SHAP analysis were added one by one to be part of the model.

### Example 13: Key Features for the Low Versus notLow Gut Microbiota Diversity Model and Associated Recommendations

For the model presented in Example 7, the top 30 features that constitute the model were shown in FIG. 6A. Both (i) and (ii) were obtained by running SHAP algorithm analyses. (i) shows the average impact per feature on the model output in their order of importance from high to low. The main/best feature was the top horizontal bar. The next best feature was the second horizontal bar and so on. (ii) shows in more details the impact of a feature per instance/sample on the model output. The color gradation from grey to black indicates low to high values for that feature. The vertical line at 0.00 defines the directionality of impact—to the left is negative impact and to the right is positive impact on the model output. Here, the SHAP analysis output was with respect to the reference class which is “Low”.

If a feature has black values towards the right of the vertical line at 0.00, this indicates higher values of this feature contribute positively to the model output. Vice a versa, if a feature has black values towards the left of the vertical line at 0.00, this indicates higher values of this feature contribute negatively to the model output. Similarly, if a feature has grey values towards the right of the vertical line at 0.00, this indicates lower values of this feature contribute positively to the model output. Vice a versa, if a feature has grey values towards the left of the vertical line at 0.00, this indicates lower values of this feature contribute negatively to the model output.

As can be seen from FIG. 6A (i), as an example, some of the important features for this model to predict Low versus notLow gut microbiota diversity were related to: antibiotics usage, alcohol—consumption, frequency, type, country of residence as USA or UK, vegetable consumption frequency, exercise frequency and so on.

Antibiotic usage history impacted the microbiome negatively as can be seen here—it was amongst the very top features used by this model (FIG. 6A (i)); lower values of Antibiotic history, i.e., if one took antibiotics recently had a positive relation with impact on model output towards the “Low” class—implying that recent antibiotics usage pushed the microbiome status to be on the lower side. Similar results were seen for Antibiotics usage history in a different Low versus notLow model presented in Example 8 (FIG. 6B (i), (ii)).

In FIG. 17, per feature, the SHAP Dependence Plot showed for each data instance/sample, the points with the feature value on the x-axis and the corresponding Shapley value on the y-axis. SHAP explained the prediction of each instance by computing the contribution of each feature to the prediction. Shapley value explanation was represented as an additive feature attribution method, as a linear model. The reference class here was “Low”, so the positive coefficients of SHAP value for the corresponding x-values of the feature indicate how much the model was affected by this feature in predicting the “Low” class.

FIG. 17(A) depicts the SHAP dependence plot for Antibiotics history. For all the individuals on recent antibiotics usage within the past one year (all data points on x-axis except the value at 500), the SHAP values were positive, indicating that this was related to being in the “Low” class of microbiome status. Similarly, only for individuals with antibiotics usage beyond one year, the SHAP values were negative, indicating that was now not related to being in “Low” but in “notLow” microbiome status.

In summary from SHAP analysis shown in FIGS. 6A (i), 6A (ii), 6B (i), 6B (ii) and 17(A), the conclusion was that recent antibiotics use adversely impacts the microbiome and pushed it to a “Low” status. Thus, the consequent advice or recommendation was if it is necessary to take antibiotics based on a physician's prescription, the recommendation of the invention would be to take other complementary solutions to boost the microbiome status both during and after antibiotics usage.

Based on similar reasoning and explanations above and looking holistically together at FIGS. 6A (i), 6A (ii), 6B (i), 6B (ii) and 17(C), it can be inferred that consumption of red wine was good for the microbiome as it was related to being in the “notLow” microbiome status. Based on these results the recommendation of the invention would be to drink red wine—occasionally (1-2 times/week), regularly (3-5 times/week) or daily.

Based on similar reasoning and explanations above and looking holistically together at FIGS. 6A (i), 6A(ii), 6B (i), 6B (ii) and 17(D), it can be inferred that residents of USA tend to have “Low” microbiome status while those of UK tend to have “notLow” microbiome status. It should be kept in mind that this was a complex multivariate analysis which will try to explain all of the data with respect to its relationship with the microbiome status but also with respect to its relation to other parts of the data. It demonstrated the importance of the geographic location of the population cohort. The recommendation of the invention would be to boost microbiome status through supplementary dietary interventions particularly for individuals located in geographically disadvantaged locations.

Based on similar reasoning and explanations above and looking holistically together at FIGS. 6A(i), 6A(ii) and 17(f), it can be inferred that vegetable consumption frequency was associated with microbiome status. Specifically, increased vegetable consumption was associated with “notLow” microbiome status. Thus, the recommendation of the invention would be to consume daily minimum 2-3 servings of vegetables in a day, including potatoes (where 1 serving=½ cup vegetables/potatoes; 1 cup leafy raw vegetables).

The final recommendation is the result of a complex multivariate analysis where features were related to each other and the final impact on the microbiome status of an individual was a combination of different factors.

The system of the present invention with its user-friendly digital interface would incorporate these recommendations to communicate them directly with the user for improving their microbiome status.

### Example 14: Key Features for the High Versus notHigh Gut Microbiota Diversity Model and Associated Recommendations

For the model presented in Example 9, the top 30 features that constitute the model were shown in FIG. 7A. Both (i) and (ii) were obtained by running SHAP algorithm analyses. For further technical explanations on the interpretations of different SHAP analyses plots, please see Example 13. Briefly, (i) shows the average impact per feature on the model output in their order of importance from high to low. (ii) shows in more details the impact of a feature per instance/sample on the model output. Here, in FIG. 7A the SHAP analysis output was with respect to the reference class which is “High”.

As can be seen from FIG. 7A (i), as an example, some of the important features for this model to predict High versus notHigh gut microbiota diversity were related to: age, country/location of residence, health status (no IBD, no Diabetes), antibiotic usage, exercise frequency, home cooked meals frequency, BMI, alcohol—frequency, consumption, type, vegetable frequency, fruit frequency and so on.

Country/location of residence, antibiotic usage, alcohol—frequency, consumption, type and vegetable frequency have been discussed in Example 13. The inferences and recommendations on these features for the improvement of microbiome status are similar as mentioned in Example 13. Briefly, the recommendation of the invention would be to boost microbiome status through supplementary dietary interventions particularly for individuals located in geographically disadvantaged locations; to take other complementary solutions to boost the microbiome status both during and after antibiotics usage; to drink red wine regularly (3-5 times/week) or daily; and to consume daily minimum 2-3 servings of vegetables in a day, including potatoes (where 1 serving=½ cup vegetables/potatoes; 1 cup leafy raw vegetables).

Details that indicate how these features impacted the microbiome to the “High” status are in FIGS. 7A (ii) and FIG. 18. Details that indicate how these features impacted the microbiome to the “notHigh” status is in FIG. 7B (ii) as the reference class here was set as “notHigh” and thus the direction of associations is towards “notHigh”. Briefly, having not used antibiotics in the past one year, regular or daily consumption of red wine, and daily consumption of vegetables was associated with a “High” microbiome status.

Some of the important features specific to the High versus notHigh model are discussed next.

Being of a healthy status had positive associations with “High” microbiome status as was inferred from FIGS. 7A (ii), 7B (ii), and 18(B). Here, healthy was defined as adults from 20 to 69 years of age, with BMI between 18.5 and 30 kg/m2 and who reported no history of inflammatory bowel disease (IBD) or diabetes, and no antibiotic use in the last year. BMI was calculated from height and weight. The information on BMI and antibiotics usage showed up as independent features too in the models. The recommendation was to consult a physician if the health status was affected by IBD, Diabetes, old age and not-normal BMI range in order to get proper medical advice as per the specific condition(s).

Exercise—frequency and location, was associated with “High” microbiome status as was interpreted from FIGS. 7A(ii), 7B(ii), 18 (G) and 18(H). The recommendation was to exercise outdoors regularly (3-5 times/week) or daily.

Fruit consumption frequency had a positive association with “High” microbiome status (FIGS. 7A(ii) and 18(J)). The recommendation was to consume at least 2-3 servings of fruit in a day (1 serving=½ cup fruit; 1 medium sized fruit; 4 oz. 100% fruit juice.)

Similarly, home cooked meals frequency had a positive association with “High” microbiome status as inferred from FIGS. 7A(ii) and 18 (K). The recommendation was to cook and consume daily home cooked meals (exclude ready-to-eat meals like boxed macaroni and cheese, ramen noodles).

The final recommendation is the result of a complex multivariate analysis where features were related to each other and the final impact on the microbiome status of an individual was a combination of different factors.

The system of the present invention with its user-friendly digital interface would incorporate these recommendations to communicate them directly with the user for improving their microbiome status.

### Example 15: Key Features for the Low Versus High Gut Microbiota Diversity Model and Associated Recommendations

For the model presented in Example 11, the top 30 features that constitute the model were shown in FIG. 8A. Both (i) and (ii) were obtained by running SHAP algorithm analyses. For further technical explanations on the interpretations of different SHAP analyses plots, please see Example 13. Briefly, (i) shows the average impact per feature on the model output in their order of importance from high to low. (ii) shows in more details the impact of a feature per instance/sample on the model output. Here, in FIG. 8A the SHAP analysis output was with respect to the reference class which is “Low”.

As can be seen from FIG. 8A (i), as an example, some of the important features for this model to predict Low versus High gut microbiota diversity were related to: health status, age, antibiotics usage, residence location, alcohol—consumption, frequency, type, exercise, BMI, race, salted snacks consumption frequency, fruits consumption frequency, home cooked meals consumption frequency, vegetables consumption frequency and so on. The specific impact of these features on the model outputs was seen in FIG. 8A (ii) and FIG. 19.

In Examples 13 and 14 discussed above, the interpretation of almost all these features with respect to their contributions in the models and associated recommendations and advise to follow for these features in order to maintain or improve the microbiome status have been mentioned in detail.

Briefly, the recommendation of the invention would be to boost microbiome status through supplementary dietary interventions particularly for individuals located in geographically disadvantaged locations; to take other complementary solutions to boost the microbiome status both during and after antibiotics usage; to drink red wine regularly (3-5 times/week) or daily; and to consume daily minimum 2-3 servings of vegetables in a day, including potatoes (where 1 serving=½ cup vegetables/potatoes; 1 cup leafy raw vegetables); to consult a physician if the health status was affected by IBD, Diabetes, old age and not-normal BMI range in order to get proper medical advice as per the specific condition(s); to exercise outdoors regularly (3-5 times/week) or daily; to consume at least 2-3 servings of fruit in a day (1 serving=½ cup fruit; 1 medium sized fruit; 4 oz. 100% fruit juice); and to cook and consume daily home cooked meals (exclude ready-to-eat meals like boxed macaroni and cheese, ramen noodles).

Salted snacks consumption frequency is an additional feature found here which negatively impacts the microbiome (FIGS. 8A (ii) and 19(I)). Our recommendation was to not to consume salted snacks (potato chips, nacho chips, corn chips, popcorn with butter, French fries etc.) or rarely (less than once/week).

The final recommendation was the result of a complex multivariate analysis where features were related to each other and the final impact on the microbiome status of an individual was a combination of different factors.

The system of the present invention with its user-friendly digital interface would incorporate these recommendations to communicate them directly with the user for improving their microbiome status.

### Example 16: Building Models to Predict Microbiome Status in MDD Data

The data in the MDD consists of over 6000 metagenomic species profiles from faecal samples, paired with over 2000 associated metadata features. Metadata used in this analysis included demographic, medical, physical activity, and diet information. Shannon diversity (natural logarithmic base) and species richness were calculated.

Initial steps were related to categorizing the alpha diversity into into low, not-low, medium and high as per some of the above-mentioned definitions. Both measures of alpha diversity were used to categorize metagenomic profiles as having one of the following levels of diversity: Low, Not-low, Medium, and High. These categories were then used in one of three different models to predict alpha diversity as a categorical variable.

A list of 289 metadata features from the MDD were evaluated as possible input features for the model. MDD was split into discovery and evaluation set. For continuous variables, a confidence interval of 95% (Z ˜=1.96) was used to remove outliers. For categorical variables, classes that were less than 5% of the cohort were aggregated and relabelled as “other”, unless all classes aggregated in which case the feature was removed from the list of selectable features and another feature used.

The MDD was divided into a discovery set (87.5% of samples) and hidden evaluation set (12.5% of samples). The discovery set was used to train, optimise, and select machine learning models. The models performing best on the discovery set were then evaluated on the hidden evaluation set. Hold-out validation ensured that the dataset would not be overfit by the models after many iterations, and that all the data was truly unseen. This set was stratified to have the same response distribution as the entire cohort after the above sanitation steps. A large number of models were generated in an iterative approach, and at each iteration the discovery set was randomly split into a training and test datasets. However, the hidden evaluation set did not change. This was to ensure as the number of iterations approaches (theoretically) infinity, the possibility of the model generalising the data randomly remains as close to 0 percent as possible.

Iteratively, 20 features were randomly selected from the set of 289 metadata features. The MDD data was prepared for these 20 features and the model discovery set was randomly split into a training (75%) and test (25%) set. The samples were filtered based on the selected 20 features. Samples with any missing metadata points from the selected feature pool were removed. Several models were trained (Neural Networks, Random Forests, Gradient Boosting etc.) on the 20 selected features and optimized by cross validation on the training set. The optimized models were evaluated on the test set. Identification of feature groups with similar predictive capabilities was done using Reinforcement Learning. These steps were repeated for different sets of 20 features selected by Reinforcement Learning, until metrics such as AUC converged to a steady-state value. Finally, evaluation of best performing models was done on the hidden evaluation set.

Given the dataset contained 289 features, and 20 features were to be selected, there would be approximately 3.46×10³⁰ (that is, nonillion) unordered combinations. To overcome this computational limit, but still ensure useful features were used, a Reinforcement Learning approach was utilized. The precision-recall AUC (AUC PR) was utilized by a Reinforcement Learning based optimization process (multi-agent Reinforcement Learning) to identify feature groups with similar predictive capabilities. The model maximized AUC PR and generated clusters of models with similar feature inputs that performed well, the reward function included a derivative of the average importance of each feature, to favor robust models. The model was implemented in Haskell and primarily utilized the ‘reinforce’ framework. After converging, models that performed well were manually checked for sensitive features that would not be suitable to ask participants.

Models were trained and evaluated for the following classification tasks: Low vs not-Low alpha diversity, Low vs High alpha diversity, and Low vs Medium vs High alpha diversity. The modelling process was repeated separately for each of these classification tasks. As mentioned above, the discovery set (87.5% of samples) was split into a training set (75%) and test set (25%) and used to train and evaluate models. All models took the randomly selected 20 features as inputs and predicted the response as categorical labels (categorized microbial diversity groups). The architecture for models included: Neural Networks (NN) implemented in PyTorch with Hyperopt and Optuna, Distributed Random Forests (DRF), optimized with H2O, and Gradient Boosted Machines (GBM) optimized with H2O. The models were all trained on the same split of data, 75% of the trimmed, sanitized data (as the remainder was dependent on how many samples were removed due to sanitation). The remaining 25% was used for test set.

Overall, Gradient Boosted Machines (GBM) performed the best over several feature sets, with Neural Networks often overfitting, and Distributed Random Forests (DRF) performing well. This was beneficial, as GBMs offer better model explainability over Neural Networks-based methods. Feature importance was extracted from the GBM and DRF models. Local interpretable model-agnostic explanations (LIME) were used to identify features important for the Neural Network models.

The best performing models were evaluated on the hidden evaluation set. In total, 32 models were evaluated on this hidden data across the span of the project. Features were inspected to ensure there was no overfitting. This also included investigating the correlation of the features with the response variables, features with features, and response variables with response variables. This included the use of Pearson correlation, F-statistics, and Chi-Square testing for the distinct types of data.

### Example 17: High Versus Low Model (MDD Data)

For the MDD data, Low was defined as samples with Shannon: 0.59-3.63 & Richness: 14-150, with Natural logarithm base for Shannon index. High was defined as samples with Shannon: 4.05-4.88 & Richness: 196-331, with Natural logarithm base for Shannon index. The best performing model for the “High-Low” task performed with an AUC of 0.837 and an AUC PR of 0.577 (FIG. 20). The Mean Per-Class Error was 0.232 and the Maximum Accuracy was 0.827. The maximum accuracy is the maximum accuracy given a particular true-positive and false-positive rate (TPR, FPR) threshold. Here, FPR=0.248, which maximized the F1-score (0.741). AUC and AUCPR were calculated on the hidden evaluation set. This model used 20 metadata features. The relative weighting of each feature is shown in FIG. 21. Features were a mix of binary (antibiotic usage), categorical (age) and continuous (BMI).

### Example 18: Low Versus not Low Model (MDD Data)

For the MDD data, Low was defined as samples with Shannon: 0.59-3.50 & Richness: 14-138, with Natural logarithm base for Shannon index. Not Low was defined as samples with Shannon: 2.09-4.88 & Richness: 57-331, with Natural logarithm base for Shannon index. The best performing model for the “Low-Not Low” task had an AUC-ROC of 0.902 (FIG. 22). The Accuracy was 0.883. The Ratio of Low:NotLow samples was 1:3.59 of total 422. Many of the 20 features (and their relative importance) overlapped with those determined to be optimal for the High-Low model in example 17, such as physical activity, height, weight, and alcohol consumption. Non-overlapping features in the Low-notLow model included stress/anxiety, vegetable serves, cat or dog ownership, overseas travel, smoking, and bloating. The relative weighting of the 20 features is shown in FIG. 23.

### Example 19: Building Ensemble Models to Predict Microbiome Status in MDD Data

Although individual models performed well on binary classification problems, such as “High-Low,” solving the unbalanced task of “Low-Medium-High” performed poorly in comparison. To overcome this, ensemble modelling (also known as stacked modelling) was utilized (FIG. 24). A binary classifier for “High vs Low” with a continuous-output model using thresholds performed the best. Other explored models included a multi-output model using the consensus decision from binary combinations of High, Medium, and Low, i.e., models for High/Medium+High/Low, Medium/Low used together.

The binary model used thresholds for the decisions that maximized F1 in training. Outputs of the model were probabilities for the two classes, for example p0 and p1. Decision thresholds were learnt by the model, for example if p0 >0.50 the result would be assigned to class 0 and 1 otherwise, when utilized with the alternative probability p1. If both thresholds failed, the sample was said to be “low-confidence” and passed to the continuous model (FIG. 24). The continuous model was trained on the same data as the “High-Low” model, which meant all medium samples were discarded. The intervals for Low samples were when both alpha diversity measures fell below the third tercile, High was similarly the highest third tercile, all intermediate samples were then labelled Medium. To ensure sample sizes were consistent, the features were kept the same; in addition, this kept the questionnaire small.

### Example 20: Low-Medium-High Model (MDD Data)

For the proprietary MDD data, Low was defined as samples with Shannon: 0.59-3.63 & Richness: 14-150, with Natural logarithm base for Shannon index. Medium was defined as samples with Shannon: 3.63-4.05 & Richness: 150-196, with Natural logarithm base for Shannon index. High was defined as samples with Shannon: 4.05-4.88 & Richness: 196-331, with Natural logarithm base for Shannon index. The ensemble modelling approach provides a model with an accuracy of 0.75 (FIG. 25). The final, previously hidden test set was unbalanced. The overlap of the quantiles between the two measures of diversity meant that most samples were Medium or intermediate (FIG. 26).

TABLE 1
20 features used in the Low-Medium-High model

| Feature | Possible Values |
|---|---|
| Physical Activity (MET) | 0-45564 |
| Height (cm) | 101-204 |
| Alcohol (grams) | 0-431.8 |
| BMI | 13-73 |
| Weight (kgs) | 30-224 |
| Servings Fruit + Veg | 0-10 |
| Depression Score | 0-42 |
| Anxiety Score | 0-40 |
| Antibiotics | True, False |
| (last 6 months) | |
| Servings Fruit | 0-300 (outlier, otherwise 12) |
| Age (years) | 18-20, 20-40, 40-60, >60 |
| Pets (Mammals) | True, False |
| Amount of Sleep Night | <4, 4-6, 6-8, >8 |
| Before (hours) | |
| Medication | True, False |
| (last 12 months) | |
| Currently taking probiotics | True, False |
| Servings Fruit + Veg, | 0-2.22, 2.22-4.44, 4.44-6.66, 6.66-10 |
| (Nominal) | |
| Pneumococcal Vaccine | Yes, No, Don't know |
| Gender | Male, Female, Other |
| Quality of Sleep Night | Terrible, Poor, Moderate, Good, |
| Before | Excellent |
| Depression Score | Normal, Moderate, Extreme |
| (Categorical) | |

1. Method for determining the gut microbiome status comprising:
(i) determining the gut microbiome status in a subject; and
(ii) providing recommendations to improve or maintain microbiome status in said subject.

2. Method according to claim 1 wherein the determination of gut microbiome status is by a questionnaire to predict the microbiome diversity of said subject.

3. Method according to claim 1 wherein the determination of gut microbiome status is additionally by a biological sample to quantify the microbiome diversity of said subject.

4. Method according to claim 1 wherein said method is computer-implemented.

5. Method according to claim 1 wherein said method involves evaluation of feature parameters related to gut microbiome status as Low, Medium or High.

6. Method according to claim 1 wherein said feature parameters related to gut microbiome status arc is selected from the group consisting of:
(i) geographic country of residence of the subject comprising specific latitude and longitude;
(ii) antibiotic use comprising whether and when antibiotics have been used in the past 1 year;
(iii) medication usage comprising whether and when medications have been used in the last 12 months;
(iv) anthropometric data comprising age, weight, height, body mass index, gender;
(v) alcohol consumption comprising the type of alcohol, amount and frequency of consumption;
(vi) smoking status
(vii) exercise and/or physical activity comprising the location of exercise as indoor or outdoor, frequency, and duration;
(viii) ethnicity;
(ix) season;
(x) travel comprising the location of travel and duration;
(xi) sleep comprising duration in hours and/or sleep quality;
(xii) stress status, anxiety status and/or depression status;
(xiii) history of diseases comprising chicken pox, irritable bowel disease, or diabetes
(xiv) history of allergies comprising seasonal or food allergies and/or food intolerances;
(xv) vaccination status comprising flu vaccine or pneumococcal vaccine.
(xvi) nutritional supplement use comprising vitamin or mineral supplements;
(xvii) source of drinking water;
(xviii) personal hygiene comprising flossing of teeth, use of deodorant, use of cosmetics; and
(xix) type of food consumption comprising intake of vegetable, fruit, fermented food and/or whole grains, amount and frequency of consumption.

7. Method according to claim 3 wherein said feature parameters related to gut microbiome status is selected from the group consisting of:
(i) geographic country of residence of the subject comprising specific latitude and longitude;
(ii) antibiotic use comprising whether and when antibiotics have been used in the past 1 year;
(iii) medication usage comprising whether and when medications have been used in the last 12 months
(iv) anthropometric data comprising age, weight, height, body mass index, gender;
(v) alcohol consumption comprising the type of alcohol, amount and frequency of consumption;
(vi) smoking status
(vii) exercise and/or physical activity comprising the location of exercise as indoor or outdoor, frequency, and duration;
(viii) ethnicity;
(ix) season;
(x) travel comprising the location of travel and duration;
(xi) sleep comprising duration in hours and/or sleep quality;
(xii) stress, anxiety status and/or depression status;
(xiii) history of diseases comprising chicken pox, irritable bowel disease; diabetes
(xiv) history of allergies comprising seasonal or food allergies and/or food intolerances;
(xv) vaccination status comprising flu vaccine or pneumococcal vaccine.
(xvi) nutritional supplement use comprising vitamin or mineral supplements;
(xvii) source of drinking water;
(xviii) personal hygiene comprising flossing of teeth, use of deodorant, use of cosmetics; and
(xix) type of food consumption comprising intake of vegetable, fruit, fermented food and/or whole grains, amount and frequency of consumption.

8. Method according to claim 1 wherein said method involves the steps of:
(i) determining at least one feature parameter related to gut microbiome status in a subject;
(ii) comparing at least one feature parameter related to gut microbiome status in said subject to a population database of subjects from the same geographical region; and
(iii) determining whether a subject is Low, Medium or High on at least one feature parameter.

9. (canceled)

10. Method according to claim 1 wherein the subject is informed of their gut microbiome status on a computer interface.

11. (canceled)

12. Method for optimizing one or more dietary interventions for a subject comprising:
(i) determining the gut microbiome-status of a subject according to a method comprising:
determining the gut microbiome status in a subject and
providing recommendations to improve or maintain microbiome status in said subject; and
(ii) applying the dietary intervention to the subject.

13. Method according to claim 12 wherein said dietary intervention comprises recommendations for food or nutrient groups selected from the group consisting of:
(i) whole grain foods;
(ii) beans and legumes;
(iii) fiber;
(iv) nuts and seeds; and
(v) omega-3 fatty acids.

14. Method according to claim 12 or 13 wherein said recommendations for food or nutrient group comprises recommendations of meal plans or recipes containing:
(i) whole grain foods;
(ii) beans and legumes;
(iii) fiber;
(iv) nuts and seeds; and
(v) omega-3 fatty acids.

15. Method according to claim 12 wherein said recommendations include lifestyle recommendations concerning exercise, alcohol consumption, sleep, dental hygiene, and nutritional supplement use.
